SEQUENCE LISTING (109 sequences)

SEQ ID NO: 1 - protein, 147 aa, Homo sapiens
MGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTAVASALSSRYH

SEQ ID NO: 2 - protein, 147 aa, Homo sapiens
MGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTGVASALSSRYH

SEQ ID NO: 3 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 4 - protein, 142 aa, Homo sapiens; identical to SEQ ID NO: 3

SEQ ID NO: 5 - DNA, 20 bp, Homo sapiens
gcaaaggaacggctacgacc

SEQ ID NO: 6 - DNA, 20 bp, Homo sapiens
ttgaaacgtggagagaccag

SEQ ID NO: 7 - DNA, 20 bp, Homo sapiens
gctgtcggaaatcatggaga

SEQ ID NO: 8 - DNA, 20 bp, Homo sapiens
gcaggatgtggaccaacgtg

SEQ ID NO: 9 - DNA, 20 bp, Homo sapiens
tgagcctgcctacctgacag

SEQ ID NO: 10 - DNA, 20 bp, Homo sapiens
cagtccgggcaagaggcgga

SEQ ID NO: 11 - DNA, 20 bp, Homo sapiens
gacttgcccctaattttcga

SEQ ID NO: 12 - DNA, 20 bp, Homo sapiens
tgcactgctggatgaagtgc

SEQ ID NO: 13 - DNA, 20 bp, Homo sapiens
ggagcgaactcgatgggcta

SEQ ID NO: 14 - DNA, 20 bp, Homo sapiens
actactggcctccaaaggaa

SEQ ID NO: 15 - DNA, 20 bp, Homo sapiens
tgtgtgagctcagggagctg

SEQ ID NO: 16 - DNA, 20 bp, Homo sapiens
gtttgtgaaggctaaagccc

SEQ ID NO: 17 - DNA, 20 bp, Homo sapiens
ggggacagcgacgatgagga

SEQ ID NO: 18 - DNA, 20 bp, Homo sapiens
atggtgcgctaccacgaggg

SEQ ID NO: 19 - DNA, 20 bp, Homo sapiens
ggagggcatgagaaagtgga

SEQ ID NO: 20 - DNA, 20 bp, Homo sapiens
ggtttagtgcgttccagggg

SEQ ID NO: 21 - DNA, 20 bp, Homo sapiens
gctgcatgaccaggtagtag

SEQ ID NO: 22 - DNA, 20 bp, Homo sapiens
ggagagctataaacagatgc

SEQ ID NO: 23 - DNA, 20 bp, Homo sapiens
tcttttgggagccaaaccca

SEQ ID NO: 24 - DNA, 20 bp, Homo sapiens
tctcaaagagctggaaactg

SEQ ID NO: 25 - DNA, 20 bp, Homo sapiens
gaggagggagatgaccacga

SEQ ID NO: 26 - DNA, 20 bp, Homo sapiens
ataaggtctcacctgaaact

SEQ ID NO: 27 - DNA, 20 bp, Homo sapiens
aggccccgcactgattgcac

SEQ ID NO: 28 - DNA, 20 bp, Homo sapiens
aataaggctccaaaagacca

SEQ ID NO: 29 - DNA, 20 bp, Homo sapiens
gcaatgccctttcagaagcg

SEQ ID NO: 30 - DNA, 20 bp, Homo sapiens
gtagacagtaatgggagcga

SEQ ID NO: 31 - DNA, 20 bp, Homo sapiens
gaggctgggaacaacagcag

SEQ ID NO: 32 - DNA, 20 bp, Homo sapiens
gtttggccacatagttgctg

SEQ ID NO: 33 - DNA, 20 bp, Homo sapiens
ggtgctgtgtcgggatgaga

SEQ ID NO: 34 - DNA, 20 bp, Homo sapiens
tcgcgatttagaagttgtgg

SEQ ID NO: 35 - DNA, 20 bp, Homo sapiens
aatgagctctgctgtgcagg

SEQ ID NO: 36 - DNA, 20 bp, Homo sapiens
gaacctggcacctcaaggat

SEQ ID NO: 37 - DNA, 20 bp, Homo sapiens
taagagccctgaggatccat

SEQ ID NO: 38 - DNA, 20 bp, Homo sapiens
gggttctgtgtcaattcccg

SEQ ID NO: 39 - DNA, 20 bp, Homo sapiens
gcccggactgggagatgaca

SEQ ID NO: 40 - DNA, 20 bp, Homo sapiens
gccatacctcccctacacca

SEQ ID NO: 41 - DNA, 20 bp, Homo sapiens
gccttgatgaagcactgcag

SEQ ID NO: 42 - DNA, 20 bp, Homo sapiens
cccgtggcagaagatggctg

SEQ ID NO: 43 - DNA, 20 bp, Homo sapiens
acatctcttgcaaacagagt

SEQ ID NO: 44 - DNA, 20 bp, Homo sapiens
ggtgtgtgaggctggaccgg

SEQ ID NO: 45 - DNA, 20 bp, Homo sapiens
ggggctgctgctgaccaagg

SEQ ID NO: 46 - DNA, 20 bp, Homo sapiens
gctcagcacagagtgcgcca

SEQ ID NO: 47 - DNA, 20 bp, Homo sapiens
caccatggacatctacgcca

SEQ ID NO: 48 - DNA, 20 bp, Homo sapiens
tgagcgggcttattccgcag

SEQ ID NO: 49 - DNA, 20 bp, Homo sapiens
gtttgtgcaaggcaaagact

SEQ ID NO: 50 - DNA, 20 bp, Homo sapiens
agtgggaagcatcattgtgt

SEQ ID NO: 51 - DNA, 20 bp, Homo sapiens
actgggctaacctaaagctg

SEQ ID NO: 52 - DNA, 20 bp, Homo sapiens
gacctttgtgagcgagctgg

SEQ ID NO: 53 - DNA, 20 bp, Homo sapiens
agatcgtacatgttcaccgg

SEQ ID NO: 54 - DNA, 20 bp, Homo sapiens
ggacactgcccaccagacag

SEQ ID NO: 55 - DNA, 20 bp, Homo sapiens
ggcacctctgagctgaggag

SEQ ID NO: 56 - DNA, 20 bp, Homo sapiens
gaagagagggccagttgtga

SEQ ID NO: 57 - DNA, 20 bp, Homo sapiens
gaggcggatggacacggacg

SEQ ID NO: 58 - DNA, 20 bp, Homo sapiens
caaattcattaagtcctccc

SEQ ID NO: 59 - DNA, 20 bp, Homo sapiens
gaggtcttcgatcagtgggg

SEQ ID NO: 60 - DNA, 20 bp, Homo sapiens
gctgggaatccctgctgcag

SEQ ID NO: 61 - DNA, 20 bp, Homo sapiens
caagcccctttgcattgaag

SEQ ID NO: 62 - DNA, 20 bp, Homo sapiens
tggcagggagtgctacactg

SEQ ID NO: 63 - DNA, 20 bp, Homo sapiens
gacaaccgccatccagactg

SEQ ID NO: 64 - DNA, 20 bp, Homo sapiens
gaaaaggctggtgaaagtta

SEQ ID NO: 65 - DNA, 20 bp, Homo sapiens
gaagaccgccctttgggagg

SEQ ID NO: 66 - DNA, 20 bp, Homo sapiens
gaagccttgcacagagcctg

SEQ ID NO: 67 - DNA, 20 bp, Homo sapiens
gagtcttgaagatatcagga

SEQ ID NO: 68 - DNA, 20 bp, Homo sapiens
gcggggtgtctacacagaga

SEQ ID NO: 69 - DNA, 20 bp, Homo sapiens
taatcagcaagcctttgatg

SEQ ID NO: 70 - DNA, 20 bp, Homo sapiens
actccgctccctgctcgccg

SEQ ID NO: 71 - DNA, 20 bp, Homo sapiens
ggagttgatgatcttgcaga

SEQ ID NO: 72 - DNA, 20 bp, Homo sapiens
gcatttgctgcaggaccccg

SEQ ID NO: 73 - DNA, 20 bp, Homo sapiens
ataatgacaagaagaacccc

SEQ ID NO: 74 - DNA, 20 bp, Homo sapiens
cctaaagcctcaaaatagga

SEQ ID NO: 75 - DNA, 20 bp, Homo sapiens
gaagccttgcacagagcctg

SEQ ID NO: 76 - DNA, 20 bp, Homo sapiens
gagtcttgaagatatcagga

SEQ ID NO: 77 - DNA, 20 bp, Homo sapiens
ttagctggcttaaaggatgg

SEQ ID NO: 78 - DNA, 20 bp, Homo sapiens
ggaatgccagtggagagcca

SEQ ID NO: 79 - DNA, 20 bp, Homo sapiens
tgaaagacaagtcgtccggg

SEQ ID NO: 80 - DNA, 20 bp, Homo sapiens
gtagcaccaactctcagcta

SEQ ID NO: 81 - DNA, 20 bp, Homo sapiens
ggccctagagaagtatggga

SEQ ID NO: 82 - DNA, 20 bp, Homo sapiens
ggaggacaagaagatgcagc

SEQ ID NO: 83 - DNA, 20 bp, Homo sapiens
gggaaatgcgcaccatgggg

SEQ ID NO: 84 - DNA, 20 bp, Homo sapiens
taagagccctgaggatccac

SEQ ID NO: 85 - DNA, 20 bp, Homo sapiens
gagctacgtggtgaaccgtg

SEQ ID NO: 86 - DNA, 20 bp, Homo sapiens
ggatgaacctatgggagagg

SEQ ID NO: 87 - DNA, 20 bp, Homo sapiens
gcagatgccagcaaacatgg

SEQ ID NO: 88 - DNA, 20 bp, Homo sapiens
aggaggagcagaaggacctg

SEQ ID NO: 89 - DNA, 20 bp, Homo sapiens
ggcacttagtaaagtcagtg

SEQ ID NO: 90 - DNA, 20 bp, Homo sapiens
aaggctgtccaacaggaata

SEQ ID NO: 91 - DNA, 20 bp, Homo sapiens
taactttagcttttgccggg

SEQ ID NO: 92 - DNA, 20 bp, Homo sapiens
cgggcatataggtttgtgca

SEQ ID NO: 93 - DNA, 20 bp, Homo sapiens
gtttgcgagtaaaccccaaa

SEQ ID NO: 94 - DNA, 20 bp, Homo sapiens
gagcatctcaaacacaacga

SEQ ID NO: 95 - DNA, 20 bp, Homo sapiens
cgagatcaagttgtacgtta

SEQ ID NO: 96 - DNA, 20 bp, Homo sapiens
tcatctacggcaaactctat

SEQ ID NO: 97 - DNA, 59 bp, Homo sapiens
ccggcacagatcaaggtagtgaaatctcgagatttcactaccttgatctgtgttttttg

SEQ ID NO: 98 - DNA, 59 bp, Homo sapiens
ccggcaaggtagtgaaattctcctactcgagtaggagaatttcactaccttgttttttg

SEQ ID NO: 99 - DNA, 59 bp, Homo sapiens
ccggcagatgagttaggaggactgtctcgagacagtcctcctaactcatctgttttttg

SEQ ID NO: 100 - DNA, 59 bp, Homo sapiens
ccggcacaaggctatcttagcagctctcgagagctgctaagatagccttgtgttttttg

SEQ ID NO: 101 - DNA, 58 bp, Homo sapiens
ccgggactatatccagggcttattgctcgagcaataagccctggatatagtctttttg

SEQ ID NO: 102 - DNA, 58 bp, Homo sapiens
ccggcgtaagaataacagtggtcttctcgagaagaccactgttattcttacgtttttg

SEQ ID NO: 103 - DNA, 58 bp, Homo sapiens
ccggcgtgtgccaaatggtttgaaactcgagtttcaaaccatttggcacacgtttttg

SEQ ID NO: 104 - DNA, 584 bp, Homo sapiens
acactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcac

SEQ ID NO: 105 - DNA, 586 bp, Homo sapiens
acactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacaca

SEQ ID NO: 106 - DNA, 577 bp, Homo sapiens
actcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggccatgcttcttgccccttgggcctccccccagcccctcctccccttcctgcacccgtacccccgtggtctttgaataaagtctgagtgggcggca

SEQ ID NO: 107 - DNA, 576 bp, Homo sapiens
actcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagccgttcctcctgcccgctgggcctcccaacgggccctcctcccctccttgcaccggcccttcctggtctttgaataaagtctgagtgggcagca

SEQ ID NO: 108 - protein, 768 aa, Homo sapiens
MSNLSKGTGSRKDTKMRIRAFPMTMDEKYVNSIWDLLKNAIQEIQRKNNSGLSFEELYRNAYTMVLHKHGEKLYTGLREVVTEHLINKVREDVLNSLNNNFLQTLNQAWNDHQTAMVMIRDILMYMDRVYVQQNNVENVYNLGLIIFRDQVVRYGCIRDHLRQTLLDMIARERKGEVVDRGAIRNACQMLMILGLEGRSVYEEDFEAPFLEMSAEFFQMESQKFLAENSASVYIKKVEARINEEIERVMHCLDKSTEEPIVKVVERELISKHMKTIVEMENSGLVHMLKNGKTEDLGCMYKLFSRVPNGLKTMCECMSSYLREQGKALVSEEGEGKNPVDYIQGLLDLKSRFDRFLLESFNNDRLFKQTIAGDFEYFLNLNSRSPEYLSLFIDDKLKKGVKGLTEQEVETILDKAMVLFRFMQEKDVFERYYKQHLARRLLTNKSVSDDSEKNMISKLKTECGCQFTSKLEGMFRDMSISNTTMDEFRQHLQATGVSLGGVDLTVRVLTTGYWPTQSATPKCNIPPAPRHAFEIFRRFYLAKHSGRQLTLQHHMGSADLNATFYGPVKKEDGSEVGVGGAQVTGSNTRKHILQVSTFQMTILMLFNNREKYTFEEIQQETDIPERELVRALQSLACGKPTQRVLTKEPKSKEIENGHIFTVNDQFTSKLHRVKIQTVAAKQGESDPERKETRQKVDDDRKHEIEAAIVRIMKSRKKMQHNVLVAEVTQQLKARFLPSPVVIKKRIEGLIEREYLARTPEDRKVYTYVA

SEQ ID NO: 109 - protein, 374 aa, Homo sapiens
MSRVPSPPPPAEMSSGPVAESWCYTQIKVVKFSYMWTINNFSFCREEMGEVIKSSTFSSGANDKLKWCLRVNPKGLDEESKDYLSLYLLLVSCPKSEVRAKFKFSILNAKGEETKAMESQRAYRFVQGKDWGFKKFIRRDFLLDEANGLLPDDKLTLFCEVSVVQDSVNISGQNTMNMVKVPECRLADELGGLWENSRFTDCCLCVAGQEFQAHKAILAARSPVFSAMFEHEMEESKKNRVEINDVEPEVFKEMMCFIYTGKAPNLDKMADDLLAAADKYALERLKVMCEDALCSNLSVENAAEILILADLHSADQLKTQAVDFINYHASDVLETSGWKSMVVSHPHLVAEAYRSLASAQCPFLGPPRKRLKQS

(19) United States
(12) Patent Application Publication
(10) Pub. No.: US 2022/0017908 A1
(43) Pub. Date: Jan. 20, 2022

(54) COMPOSITIONS AND METHODS FOR INCREASING FETAL HEMOGLOBIN AND TREATING SICKLE CELL DISEASE

(71) Applicant: Fulcrum Therapeutics, Inc., Cambridge, MA (US)

(72) Inventors: Peter Rahl, Cambridge, MA (US); Angela Marie Cacace, Cambridge, MA (US); Michael Cameron, Cambridge, MA (US); Akshay Kakumanu, Cambridge, MA (US)

(21) Appl. No.: 17/295,782

(22) PCT Filed: Nov. 20, 2019
(86) PCT No.: PCT/US2019/062461

§ 371 Date: May 20, 2021

Related U.S. Application Data
(60) Provisional application No. 62/769,796, filed on Nov. 20, 2018.

(51) Int. Cl. C12N 15/67; A61K 31/7105; C12N 15/11; C12N 9/22; A61K 38/46

(52) U.S. Cl. CPC C12N 15/67; A61K 31/7105; C12N 2310/20; C12N 9/22; A61K 38/465; C12N 15/11

(57) ABSTRACT

The present invention relates to compositions and methods of increasing levels of fetal hemoglobin (HbF) in cells. The present invention further relates to methods for treating patients suffering from blood cell diseases, including those associated with reduced amounts of functional adult hemoglobin (HbA), such as sickle cell disease and β-thalassemias.

### CROSS REFERENCE TO RELATED APPLICATIONS

This application claims the benefit of, and priority to, U.S. Provisional Application No. 62/769,796, filed on Nov. 20, 2018, the contents of which is incorporated herein by reference in their entireties.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is FULC_033_01WO_ST25.txt. The text file is 33 KB, was created on Nov. 20, 2019, and is being submitted electronically via EFS-Web.

### FIELD OF THE DISCLOSURE

The present disclosure relates to targets, compositions and methods of inducing fetal hemoglobin (hemoglobin γ (HBγ) or HbF) expression in erythroid cells. The present disclosure further relates to methods for treating patients suffering from diseases associated with blood cell disorders, such as Sickle Cell Disease (SCD) or β-thalassemias, including those where elevated expression of HbF protein can compensate for a mutant or defective hemoglobin β (HBB) gene, a mutant or defective HBB protein, or changes in HBB protein levels.

### BACKGROUND

Hemoglobin is the critical protein involved in oxygen transport throughout the body of vertebrates. It is found in red blood cells and consists of two a subunits and two β-like subunits.

The composition of hemoglobin is developmentally regulated, and the human genome encodes multiple versions of these proteins, which are expressed during distinct stages of development (Blobel et al, Exp Hematol 2015; Stamatoyannopoulos G. Exp Hematol 2005). In general, fetal hemoglobin (HbF) is composed of two subunits of hemoglobin γ (HBγ) and two subunits of hemoglobin α (HBα) and adult hemoglobin (HbA) is composed of two subunits of hemoglobin β (HBβ) and two subunits of HBα. Thus, the β-like subunit utilized during the fetal stage of development (HBγ) switches to hemoglobin β (HBβ) after birth.

The developmental regulation of the expression of β-like subunits has been the focus of intense studies for decades (Li et al. Blood 2002). All five β-like subunits in humans reside on chromosome 11, where their genomic location corresponds to their temporal expression pattern. A distal cluster of enhancer elements, called the locus control region (LCR), coordinates the expression pattern at the β globin locus, where multiple transcription factors, including GATA1, GATA2, KLF1, KLF2, and MYB and TAL1, bind at specific locations within the LCR at specific times in development. The five human β-like subunits are epsilon (HBE1; ε), gammaG (HBG2; γ), gammaA (HBG1; γ), delta (HBD; δ) and beta (HBB; β). The HBE1 gene is expressed during embryonic development, the HBG1 and HBG2 genes are expression during fetal development, and HBD and HBB genes are expressed in adults. The HBG1 and HBG2 genes encode identical proteins except for a single amino acid change at residue 136 (HBG1=gly; HBG2=ala). Red blood cell disorders like Sickle Cell Disease (SCD) and β-thalassemias are caused by alterations within the gene for the hemoglobin β (HBβ) subunit.

SCD affects millions of people worldwide and is the most common inherited blood disorder in the United States (70.000-80,000 Americans). SCD has a high incidence in African Americans, where it is estimated to occur in 1 in 500 individuals. SCD is an autosomal recessive disease caused by single homozygous mutations in both copies of the HBB gene (E6V) that result in a mutant hemoglobin protein called HbS (https://ghr.nlm.nih.gov/condition/sickle-cell-disease). Under deoxygenated conditions, the HbS protein polymerizes, which leads to abnormal red blood cell morphology. This abnormal morphology can lead to multiple pathologic symptoms including vaso-occlusion, pain crises, pulmonary hypertension, organ damage and stroke.

β-thalassemia is caused by mutations in the HBB gene and results in reduced hemoglobin production (https://ghr.nlm.nih.gov/condition/beta-thalassemia). The mutations in the HBB gene typically reduce the production of adult β-globin protein, which leads to low levels of adult hemoglobin, HbA. This leads to a shortage of red blood cells and a lack of oxygen distribution throughout the body. Patients with β-thalassemias can have weakness, fatigue and are at risk of developing abnormal blood clots. Thousands of infants are born with β-thalassemia each year, and symptoms are typically detected within the first two years of life.

The identification of factors that regulate the expression of fetal hemoglobin could be useful targets for the treatment of SCD and β-thalassemias, since upregulation of fetal hemoglobin could compensate for mutant HbS protein in SCD or a lack of HbA in β-thalassemias. Because β-like globin expression is developmentally regulated, with a reduction in the fetal ortholog (γ) occurring shortly after birth concomitantly with an increase in the adult ortholog (β), it has been postulated that maintaining expression of the anti-sickling γ ortholog may be of therapeutic benefit in both children and adults. A fetal ortholog of HBβ, hemoglobin γ (HBγ) can reverse disease-related pathophysiology in these disorders by also forming complexes with the required hemoglobin α subunit (Paikari and Sheehan, Br J Haematol 2018; Lettre and Bauer, Lancet 2016). Expression of the fetal hemoglobin protein can reverse the SCD pathophysiology through inhibiting HbS polymerization and morphologically defective red blood cells. Functionally, upregulation of either the HBG1 or HBG2 gene can compensate for mutant or defective adult HBβ. Based on clinical and preclinical studies, upregulation of hemoglobin γ (HBγ) is the proposed mechanism for compounds including Palmolidomide and Hydroxyurea and targets including EHMT1/EHMT2 and LSD1 (Moutouh-de Parseval et al. J Clin Invest 2008; Letvin et al. NEJM 1984; Renneville et al. Blood 2015; Shi et al. Nature Med 2015).

Given the severity and lack of effective treatments for blood cell disorders, such as Sickle Cell Disease (SCD) and β-thalassemias, including those where elevated expression of HbF protein could compensate for a mutant or defective hemoglobin β (HBβ) gene, there is clearly a need for new methods of treatment for these disorders. The present disclosure meets this need by providing new therapeutic agents and methods for increasing HbF for the treatment of these disorders.

### SUMMARY OF THE INVENTION

The present disclosure is based, in part, on the identification of novel targets for inducing fetal hemoglobin (hemoglobin γ (HBγ) or HbF) expression in erythroid cells. The present disclosure further relates to methods for treating patients suffering from diseases associated with blood cell disorders, such as Sickle Cell Disease (SCD) or β-thalassemias.

In one embodiment, the present disclosure provides a method for increasing expression of a fetal hemoglobin (HbF) in a cell, comprising contacting a cell with an inhibitor of a target protein or protein complex that functions to regulate HbF expression. In some embodiments, the HbF comprises hemoglobin gamma and hemoglobin alpha. In some embodiments, the hemoglobin gamma comprises hemoglobin gamma G1 (HBG1) and/or or hemoglobin gamma G2 (HBG2). In particular embodiments, the target protein or protein complex regulates HbF expression via a molecular signaling pathway listed in Table 5. In particular embodiments, the molecular signaling pathway is selected from the group consisting of: glucagon signaling pathway, carbon metabolism, oxytocin signaling, glycolysis, gluconeogenesis, endocrine resistance, Gonadotropin-releasing hormone (GnRH) signaling, oocyte meiosis, fatty acid degradation, and inflammatory mediator regulation of Transient Receptor Potential (TRP) channels. In certain embodiments, the target protein is CUL3. In certain embodiments, the target protein is SPOP. In certain embodiments, the target protein is selected from those listed in Table 1, Table 2, Table 3, Table 4, Table 5, Table 6 or Table 7. In certain embodiments, the hit shows enriched expression in whole blood versus other tissues and cell types. In certain embodiments, the target protein (or hit) is expressed in late stage erythroid cells or listed in Table 7. In some embodiments, the target protein is permanently or transiently associated with a multi-protein complex that regulates HbF expression. In some embodiments, the multi-protein complex is selected from those listed in Table 3 or Table 4, and the target is selected from those listed in Table 3 or Table 4. In certain embodiments, CUL3 is permanently or transiently associated with the multi-protein complex. In certain embodiments, the multi-protein complex is selected from D(4) dopamine receptor (DRD4)-Kelch like protein 12 (KLH12)-CUL3, ubiquitin E3 ligase, coiled coil domain containing protein 22 (CCDC22)-COMM domain containing protein 8 (COMMD8)-CUL3, or Cullin associated NEDD8 dissociated protein (CAND1)-CUL3-E3 ubiquitin protein ligase RBX1 (RBX). In certain embodiments, SPOP is permanently or transiently associated with the multi-protein complex. In certain embodiments, the multi-protein complex is a ubiquitin E3 ligase complex. In particular embodiments, the inhibitor targets a nucleotide sequence encoding the target protein or protein complex thereby inhibiting or preventing the expression of the target protein or protein complex. In some embodiments, the nucleotide sequence encoding the target protein or protein complex is DNA or RNA. In certain embodiments, the nucleotide sequence encodes CUL3, and optionally comprises or consists of a nucleic acid encoding the amino acid sequence of SEQ ID NO: 108. In certain embodiments, the nucleotide sequence encodes SPOP, and optionally comprises or consists of a nucleic acid encoding the amino acid sequence of SEQ ID NO: 109. In some embodiment, the inhibitor is selected from a group consisting of: a small molecule, a nucleic acid, a polypeptide, and a nucleoprotein complex, e.g., which bind to a target protein or a polynucleotide sequence encoding the target protein, such as a gene or mRNA encoding the target protein. It should be understood that an inhibitor or a target protein may inhibit the target protein by inhibiting the target protein directly, e.g., by binding to the target protein, or by inhibiting expression of the target protein, e.g., by binding to a polynucleotide encoding the target protein. In some embodiments, the nucleic acid is selected from DNA, RNA, shRNA, siRNA, microRNA, gRNA, and antisense oligonucleotide. In certain embodiments, the polypeptide is selected from a protein, a peptide, a protein mimetic, a peptidomimetic, an antibody or functional fragment thereof, and an antibody-drug conjugate or a functional fragment thereof. In particular embodiments, the nucleoprotein complex is a ribonucleoprotein complex (RNP) comprising: a) a first sequence comprising a guide RNA (gRNA) that specifically binds a target sequence, wherein the target sequence comprises a regulator of HbF expression and b) a second sequence encoding a CRISPR-Cas protein wherein the CRISPR-Cas protein comprises a DNA-nuclease activity. In particular embodiments, the cell is a blood cell, e.g., an erythrocyte. In certain embodiments, the contacting a cell occurs in vitro, in vivo, ex vivo, or in situ.

In a related embodiment, the disclosure provides a pharmaceutical composition for increasing expression of fetal hemoglobin (HbF) comprising: an inhibitor of a target protein or protein complex that functions to regulate HbF expression, and a diluent, excipient, and carrier formulated for delivery to a patient in need thereof. In particular embodiments, the inhibitor is a small molecule, a nucleic acid, e.g., DNA, RNA, shRNA, siRNA, microRNA, gRNA, or antisense oligonucleotide, or a polypeptide, e.g., a protein, a peptide, a protein mimetic, a peptidomimetic, an antibody or functional fragment thereof, or antibody-drug conjugate or a functional fragment thereof. In some embodiments, the small molecule inhibitor targets CUL3. In some embodiments, the CUL3 small molecule inhibitor is selected from MLN4924, suramin, or DI-591. In some embodiments, the polypeptide specifically binds a regulator of HbF expression. In certain embodiments, the inhibitor is a ribonucleoprotein (RNP) complex comprising: a) a first sequence comprising a guide RNA (gRNA) that specifically binds a target sequence, wherein the target sequence comprises a regulator of HbF expression and b) a second sequence encoding a CRISPR-Cas protein wherein the CRISPR-Cas protein comprises a DNA-nuclease activity. In certain embodiments, the gRNA binds a gene encoding the regulator of HbF expression. In certain embodiments, the target sequence is listed in any of Tables 1, 3-4, or 6-7. In some embodiments, the gRNA comprises any one of the targets or sequences in Table 2, or a fragment thereof, or an antisense sequence of the target sequence or fragment thereof. In some embodiments, the target sequence is CUL3. In some embodiments, wherein the target sequence is SPOP. In some embodiments, the gRNA comprises any one of the sequences disclosed in Table 2. In some embodiments, the gRNA binds a gene encoding CUL3, and optionally comprises or consists of GAGCATCTCAAACACAACGA (SEQ ID NO: 94), CGAGATCAAGTTGTACGTTA (SEQ ID NO: 95), or TCATCTACGGCAAACTCTAT (SEQ ID NO: 96). In some embodiments, the gRNA binds a gene encoding SPOP, and optionally comprises or consists of TAACTTTAGCTTTTGCCGGG (SEQ ID NO: 91), CGGGCATATAGGTTTTGTGCA (SEQ ID NO: 92), or GTTTGCGAGTAAACCCCAAA (SEQ ID NO: 93). In certain embodiments, the first sequence comprising the gRNA comprises a sequence encoding a promoter capable of expressing the gRNA in a eukaryotic cell. In some embodiments, the second sequence comprising the CRISPR-Cas protein comprises a sequence capable of expressing the CRISPR-Cas protein in a eukaryotic cell, e.g., a mammalian cell, such as a blood cell, e.g., an erythrocyte. In some embodiments, the composition is delivered via a vector, e.g., a viral vector, such as an AAV.

In another related embodiment, the disclosure provides a method of treating a disease or disorder associated with a defect in a hemoglobin protein activity or expression, comprising providing to a subject in need thereof the composition disclosed herein. In some embodiments, the disease or disorder is a blood disorder, e.g., Sickle cell disease, β-thalassemia, β-thalessemia intermedia, β-thalessemia major, β-thalessemia minor, and Cooley's anemia. In some embodiments, the hemoglobin protein is selected from hemoglobin-alpha and hemoglobin-beta. In certain embodiments, the defect in the hemoglobin protein activity or expression results from a mutation, substitution, deletion, insertion, frameshift, inversion, or transposition to a nucleotide sequence which encodes the hemoglobin protein.

### BRIEF DESCRIPTION OF THE DRAWINGS

FIG. 1 is a schematic detailing the CRISPR pooled screen sample collection process. Samples were collected following puromycin selection (1), prior to FACs sorting (2) and after sorting for HbF high cells (3).

FIG. 2 provides FACS sorting plots from the CRISPR screen with Library #1. FACs plots are shown for HUDEP2 cells with control sgGFP (dark gray) and CRISPR Library #1 (light gray). The left panel plots the level of HbF (X-axis) and β-Actin (Y-axis) for each event and the line “L” indicates the HbF threshold for HbF high cells. The right panel represents the same data in a one-dimensional plot showing the HbF levels (X-axis) and Events (Y-axis) and the line “C” indicates the HbF threshold for HbF high cells. Any cell above the HbF threshold was collected in the HbF high population.

FIG. 3 provides FACS sorting plots from the CRISPR screen with Library #2. FACs plots are shown for HUDEP2 cells with control sgGFP (dark gray) and CRISPR Library #2 (light gray). The left panel plots the level of HbF (X-axis) and β-Actin (Y-axis) for each event and the line “L” indicates the HbF threshold for HbF high cells. The right panel represents the same data in a one-dimensional plot showing the HbF levels (X-axis) and Events (Y-axis) line “C” indicates the HbF threshold for HbF high cells. Any cell above the HbF threshold was collected in the HbF high population.

FIG. 4A details a list of all bioinformatics analysis performed on the CRISPR screen data: Genome alignment (left panel), hit quantification (middle panel) and hit prioritization (right panel).

FIG. 4B is a series of plots showing the distribution of guide abundance in different samples across two different screening libraries (Library #1, left; Library #2, right). Arrow indicate the peaks for the number of guides with a given abundance level at input, post-selection and following HBF+ve (HbF high positive sorted population).

FIG. 4C is a plot showing the distribution of z-score differences across samples for the Library #1. Squares indicate hits that help differentiation, and triangles indicate hits that impede differentiation.

FIG. 5A is a heatmap showing all genes that have more than one enriched gRNA in initial Library #1 screening data.

FIG. 5B is a plot detailing the overlap between Library #1 and Library #2. The triangles correspond to genes that were called hits in both the screening libraries.

FIG. 5C is an exemplary graph displaying Z-score (γ-axis) vs. UBE2H gene locus (x-axis), indicating that 4 out of the 10 designed guides RNAs have a Z-score greater than 2.5.

FIG. 6 is chart detailing the number of hits for each of the indicated distinct biological complexes. Complex membership information was taken from the CORUM database.

FIG. 7A is a heatmap showing the expression z-score of CRISPR hits enriched in whole blood (32 out of 307 hits show highly enriched expression in whole blood versus other tissues and cell types, data source: GTEx). The 32 hits showing highly enriched expression in whole blood are listed in Table 7.

FIG. 7B is a heatmap showing hits with “Late Erythroid” expression pattern (data source: DMAP). Hits with “Late Erythroid” expression include: CUL3, SAP130, PRPS1, NAP1L4, GCLC, CUL4A, GCDH, NEK1, HIRA, MST1, SPOP, GOLGA5, AUH, MAST3, CDKN1B, UBR2, MAP4K4, TAF10, HDGF, YWHAE, AMD1, EID1, HIF1AN, CDK8, DCK, FXR2, UQCRC1, TESK2, ADCK2, USP21, CAMK2D, FGFR1, PHC2, UBE2H, BPGM, SIRT2, SIRT3, NFYC, and CPT2.

FIG. 7C is a hierarchical differentiation tree of UBE2H with exemplary “Late Erythroid” expression pattern.

FIG. 8A is a series of images depicting HbF levels determined by HbF immunocytochemistry (ICC) using CRISPR Cas9-RNP-based loss of function. Cas9-RNP complexes were electroporated into proliferating CD34+ cells. Cells were then differentiated for 7 days down the erythroid lineage and HbF levels were quantified using HbF ICC. The percent F cells (top row) and mean HbF intensity (bottom row) were quantified for negative control, sgBCL11A, sgSPOP and sgCUL3.

FIGS. 8B-8E is a series of graph depicting HbF levels determined by HbF ICC using shRNA-based loss of function. Percent F cells (FIG. 8B and FIG. 8D) and mean HbF intensity (FIG. 8C and FIG. 8E) were quantified for individual shRNA constructs for negative control, shBCL11A, shSPOP and shCUL3.

### DETAILED DESCRIPTION OF THE INVENTION

The present invention relates to targets, compositions and methods for increasing fetal hemoglobin (HbF) in erythroid cells, e.g., by increasing expression of hemoglobin γ (HBγ). This can occur through upregulation of hemoglobin γ mRNA levels (e.g., HBG1 or HBG2) and/or upregulation of fetal hemoglobin protein (HBγ) levels, which results in an elevation in HbF. The targets, compositions or methods can be used alone or in combination with another agent that upregulates HbF or targets symptoms of SCD or β-thalassemia, including but not limited to, vaso-occlusion and anemia.

### Abbreviations

As used in this specification and the appended claims, the singular forms “a,” “an” and “the” include plural references unless the content clearly dictates otherwise.

As used in this specification, the term “and/or” is used in this disclosure to either “and” or “or” unless indicated otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration” refers herein to introducing an agent or composition into a subject or contacting an agent or composition with a cell and/or tissue.

Methods and Compositions

In one aspect, the present disclosure provides methods for increasing the amount of fetal hemoglobin (HbF) in a cell. In particular embodiments, the method comprises increasing expression of one or more components of HbF in a cell. In particular embodiments, the component of HbF is a hemoglobin γ (HBγ), e.g., human hemoglobin subunit gamma-1 (HBG1) or human hemoglobin subunit gamma-2 (HBG2). In particular embodiments, the component of fetal hemoglobin is a hemoglobin α (HBα), e.g., human hemoglobin subunit alpha-1 (HBA1) or human hemoglobin subunit alpha-2 (HBA2). In certain embodiments, expression of both HBγ and HBα is increased.

In certain embodiments, the fetal hemoglobin comprises a human hemoglobin subunit gamma-1 (HBG1) having the protein sequence set forth in NCBI Reference Sequence: NP_000550.2 and shown below:

(SEQ ID NO: 1)
MGHFTEEDKATITSLWGKVNVEDAGGET
LGRLLVVYPWTQRFFDSFGNLSSASAIM
GNPKVKAHGKKVLTSLGDAIKHLDDLKG
TFAQLSELHCDKLHVDPENFKLLGNVLV
TVEAIHFGKEFTPEVQASWQKMVTAVAS
ALSSRYH.

In certain embodiments, the HBG1 protein is encoded by the polynucleotide sequence set forth in NCBI Reference Sequence: NM_000559.2 and shown below:

(SEQ ID NO: 104)

| | |
|---|---|
| 1 | acactcgctt ctggaacgtc tgaggttatc |
| | aataagctcc tagtccagac gccatgagtc |
| 61 | atttcacaga ggaggacaag gctactatca |
| | caagcctgtg gggcaaggtg aatgtggaag |
| 121 | atgctggagg agaaaccctg ggaaggctcc |
| | tggttgtcta cccatggacc cagaggttct |
| 131 | ttgacagctt tggcaacctg tcctctgcct |
| | ctgccatcat aggcaacccc aaagtcaagg |
| 241 | cacatggcaa gaaggtgctg acttccttgg |
| | gagatgccac aaagcacctg gatgatctca |
| 301 | agggcacctt tgcccagctg agtgaactgc |
| | actgtgacaa gctgcatgtg gatcctgaga |
| 361 | acttcaagct cctgggaaat gtgctggtga |
| | ccgttttggc aatccatttc ggcaaagaat |
| 421 | tcacccctga ggtgcaggct tcctggcaga |
| | agatggtgac tgcagtggcc agtgccctgt |
| 481 | cctccagata ccactgagct cactgcccat |
| | gattcagagc tttcaaggat aggctttatt |
| 541 | ctgcaagcaa tacaaataat aaatctattc |
| | tgctgagaga tcac. |

In certain embodiments, the fetal hemoglobin comprises a human hemoglobin subunit gamma-2 (HBG2) having the protein sequence set forth in NCBI Reference Sequence: NP 000175.1 and shown below:

(SEQ ID NO: 2)
MGHFTEEDKATITSLWGKVNVEDAGGET
LGRLLVVYPWTQRFFDSFGNLSSASAIM
GNPKVKAHGKKVLTSLGDAIKHLDDLKG
TFAQLSELHCDKLHVDPENFKLLGNVLV
TVLAIHFGKEFTPEVQASWQKMVTGVAS
ALSSRYH.

In certain embodiments, the HBG2 protein is encoded by the polynucleotide sequence set forth in NCBI Reference Sequence: NM_000184.2, NCBI Reference Sequence: NM_000184.3, or shown below:

(SEQ ID NO: 105)

| | |
|---|---|
| 1 | acactcgctt ctggaacgtc tgaggttatc |
| | aataagcccc tagtccagac gccatgggtc |
| 61 | atttcacaga ggaggacaag gctactatca |
| | caagcctgtg gggcaaggtg aatgtggaag |
| 121 | atgctqgagg agaaaccctg ggaaggctcc |
| | tggttgtcta cccatggacc cagagqttct |
| 181 | ttgacagctt tggcaacctg tcctctgcct |
| | ctgccatcat gggcaacccc aaagtcaagg |
| 241 | cacatggcaa gaaggtgctg acttccttgg |
| | gagatgccat aaagcacctg gatgatctca |
| 301 | agggcacctt tgcccagctg agtgaactgc |
| | actgtgacaa gctgcatgtg gatcctgaga |
| 361 | acttcaagct cctgggaaat gtgctggtga |
| | ccgttttggc aatccatttc ggcaaagaat |
| 421 | tcacccctga ggtgcaggct tcctggcaga |
| | aaatggtgac tggagtggcc agtgccctgt |
| 481 | cctccagata ccactgagct cactgcccat |
| | gatgcagagc tttcaaggat aggctttatt |
| 541 | ctgcaagcaa tcaaataata aatctattct |
| | gctaagagat cacaca. |

In certain embodiments, the fetal hemoglobin comprises a human hemoglobin subunit alpha-1 (HBA11) having the protein sequence set forth in NCBI Reference Sequence: NP_000549.1 and shown below:

(SEQ ID NO: 3)
MVLSPADKTNVKAAWGKVGAHAGEYGAEALERMFLSFP
TTKTYFPHFDLSHGSAQVKGHGKKVADALTNAVAHVDD
MPNALSALSDLHAHKLRVDPVNFKLLSHCLLVTLAAHL
PAEFTPAVHASLDKFLASVSTVLTSKYR.

In certain embodiments, the HBA1 protein is encoded by the polynucleotide sequence set forth in NCBI Reference Sequence: NM_000558.4, NCBI Reference Sequence: NM_000558.5, or shown below:

(SEQ ID NO: 106)

| | |
|---|---|
| 1 | actcttctgg tccccacaga ctcagagaga |
| | acccaccatg gtgctgtctc ctgccgacaa |
| 61 | gaccaacgtc aaggccgcct ggggtaaggt |
| | cggcgcgcac gctggcgagt atggtgcgga |
| 121 | ggccctggag aggatgttcc tgtccttccc |
| | caccaccaag acctacttcc cgcacttcga |
| 131 | cctgagccac ggctctgccc aggttaaggg |
| | ccacggcaag aaggtggccg acgcgctgac |
| 241 | caacgccgtg gcgcacgtgg acgacatgcc |
| | caacgcgctg tccgccctga gcgacctgca |
| 301 | cgcgcacaag cttcgggtgg acccggtcaa |
| | cttcaagctc ctaagccact gcctgctggt |
| 361 | gaccctggcc gcccacctcc ccgccgagtt |
| | cacccctgcg gtgcacgcct ccctggacaa |
| 421 | gttcctggct tctgtgagca ccgtgctgac |
| | ctccaaatac cgttaagctg gagcctcggt |
| 481 | ggccatgctt cttgcccctt gggcctcccc |
| | ccagcccctc ctccccttcc tgcacccgta |
| 541 | cccccgtggt ctttgaataa agtctgagtg |
| | ggcggca. |

In certain embodiments, the fetal hemoglobin comprises a human hemoglobin subunit alpha-2 (HBA2) having the protein sequence set forth in NCBI Reference Sequence: NP_000508.1 and shown below:

(SEQ ID NO: 4)
MVLSPADKTNVKAAWGKVGAHAGEYGAEALERMFLSFPT
TKTYFPHFDLSHGSAQVKGHGKKVADALTNAVAHVDDMP
NALSALSDLHAHKLRVDPVNFKLLSHCLLVTLAAHLPAE
FTPAVHASLDKFLASVSTVLTSKYR.

In certain embodiments, the HBA2 protein is encoded by the polynucleotide sequences set forth in NCBI Reference Sequence: NM_000517.4, NCBI Reference Sequence: NM_000517.6, or shown below:

(SEQ ID NO: 107)

| | |
|---|---|
| 1 | actcttctgg tccccacaga ctcagagaga |
| | acccaccatg gtgctgtctc ctgccgacaa |
| 61 | gaccaacgtc aaggccgcct ggggtaaggt |
| | cggcgcgcac gctggcgagt atqgtgcgga |
| 121 | ggccctggag aggatgttcc tgtccttccc |
| | caccaccaag acctacttcc cgcacttcga |
| 181 | cctgagccac ggctctgccc aggttaaggg |
| | ccacggcaag aaggtggccg acgcgctgac |
| 241 | caacgccgtg gcgcacgtgg acgacatgcc |
| | caacgcgctg tccgccctga gcgacctgca |
| 301 | cgcgcacaag cttcgggtgg acccggtcaa |
| | cttcaagctc ctaagccact gcctgctggt |
| 361 | gaccctggcc gcccacctcc ccgccgagtt |
| | cacccctgcg gtgcacgcct ccctggacaa |
| 421 | gttcctggct tctgtgagca ccgtgctgac |
| | ctccaaatac cgttaagctg gagcctcggt |
| 481 | agccgttcct cctgcccgct gggcctccca |
| | acgggccctc ctcccctcct tgcaccggcc |
| 541 | cttcctggtc tttgaataaa gtctgagtgg |
| | gcagca. |

In certain embodiments, the fetal hemoglobin comprises two HBG1 and/or HBG2 proteins and two HBA1 and/or HBA2 proteins.

The methods disclosed herein may be practiced in vitro or in vivo.

The methods disclosed herein comprise contacting a cell with an inhibitor of a target gene, mRNA or protein (which may collectively be referred to as “target”) disclosed herein, wherein inhibition of the target results in an increased amount of fetal hemoglobin in the cell, e.g., an erythroid or red blood cell. In particular embodiments, inhibition of the target results in an increased amount of HBG1 or HBG2 in the cell. In particular embodiments, an amount of the inhibitor effective to result in increased levels of Hbγ and/or HbF is used. In particular embodiments, the methods comprise contacting a tissue, organ or organism, e.g., a mammal, with the inhibitor. In certain embodiments, one or more inhibitors, each targeting the same or different targets, may be used.

In certain embodiments, the target gene, mRNA, or protein is Cullin 3 (CUL3). CUL3 is a core component of multiple E3 ubiquitin ligase protein complexes that regulate the ubiquitination of target proteins leading to proteasomal degradation. In some embodiments, CUL3-E3 ubiquitin ligase complexes regulate multiple cellular processes responsible for protein trafficking, stress response, cell cycle regulation, signal transduction, protein quality control, transcription, and DNA replication.

In one aspect, the present disclosure provides methods for increasing the amount of fetal hemoglobin (HbF) in a cell by inhibiting or modulating the expression of CUL3.

In certain embodiments, CUL3 comprises the protein sequence:

(SEQ ID NO: 108)
MSNLSKGTGSRKDTKMRIRAFPMTMDEKYVNSIWD
LLKNAIQEIQRKNNSGLSFEELYRNAYTMVLHKHG
EKLYTGLREVVTEHLINKVREDVLNSLNNNFLQTL
NQAWNDHQTAMVMIRDILMYMDRVYVQQNNVENVY
NLGLIIFRDQVVRYGCIRDHLRQTLLDMIARERKG
EVVDRGAIRNACQMLMILGLEGRSVYEEDFEAPFL
EMSAEFFQMESQKFLAENSASVYIKKVEARINEEI
ERVMHCLDKSTEEPIVKVVERELISKHMKTIVEME
NSGLVHMLKNGKTEDLGCMYKLFSRVPNGLKTMCE
CMSSYLREQGKALVSEEGEGKNPVDYIQGLLDLKS
RFDRFLLESFNNDRLFKQTIAGDFEYFLNLNSRSP
EYLSLFIDDKLKKGVKGLTEQEVETILDKAMVLFR
FMQEKDVFERYYKQHLARRLLTNKSVSDDSEKNMI
SKLKTECGCQFTSKLEGMFRDMSISNTTMDEFRQH
LQATGVSLGGVDLTVRVLTTGYWTTQSATPKCNIP
PAPRHAFEIFRRFYXAKHSGRQLTLQHHMGSADLN
ATFYGPVKKEDGSEVGVGGAQVTGSNTRKHILQVS
TFQMTILMLFNNREKYTFEEIQQETDIPERELVRA
LQSLACGKPTQRVLTKEPKSKEIENGHIFTVNDQF
TSKLHRVKIQTVAAKQGESDPERKETRQKVDDDRK
IIEIEAAIVRIMKSRKKMQHNVLVAEVTQQLKARF
LPSPVVIKKRIEGLIEREYLARTPEDRKVYTYVA.

In certain embodiments, the target gene, mRNA, or protein is Speckle-type POZ protein (SPOP). In certain embodiments, SPOP is associated with multiple E3 ubiquitin ligase complexes.

In one aspect, the present disclosure provides methods for increasing the amount of fetal hemoglobin (HbF) in a cell by inhibiting or modulating the expression of SPOP.

In certain embodiments, SPOP comprises the protein sequence:

(SEQ ID NO: 109)
MSRVPSPPPPAEMSSGPVAESWCYTQIKVVKFSYM
WTINNFSFCREEMGEVIKSSTFSSGANDKLKWCLR
VNPKGLDEESKDYLSLYLLLVSCPKSEVRAKFKFS
ILNAKGEETKAMESQRAYRFVQGKDWGFKKFIRRD
FLLDEANGLLPDDKLTLFCEVSVVQDSVNISGQNT
MNMVKVPECRLADELGGLWENSRFTDCCLCVAGQE
FQAHKAILAARSPVFSAMFEHEMEESKKNRVEIND
VEPEVFKEMMCFIYTGKAPNLDKMADDLLAAADKY
ALERLKVMCEDALCSNLSVENAAEILILADLFISA
DQLKTQAVDFINYFIASDVLETSGWKSMIVVSHPH
LVAEAYRSLASAQCPFLGPPRKRLKQS.

The term “inhibitor” may refer to any agent that inhibits the expression or activity of a target gene, mRNA and/or protein in a cell, tissue, organ, or subject. The expression level or activity of target mRNA and/or protein in a cell may be reduced via a variety of means, including but not limited to reducing the total amount of target protein or inhibiting one or more activity of the target protein. In various embodiments, an inhibitor may inhibit the expression of a target gene, target mRNA, or a target protein, and/or an inhibitor may inhibit a biological activity of a target protein. In certain embodiments, the biological activity is kinase activity. For example, an inhibitor may competitively bind to the ATP-binding site of a kinase and inhibit its kinase activity, or it may allosterically block the kinase activity. In certain embodiments, an inhibitor causes increased degradation of a target protein. In particular embodiments, the inhibitor inhibits any of the target genes or proteins identified in Table 1, Table 2, Table 6, Table 7, Table 8, or Table 9, or any component or subunit of any of the complexes identified in Table 3 or Table 4 or pathways identified in Table 5. Methods for determining the expression level or the activity of a target gene or polypeptide are known in the art and include, e.g., RT-PCR and FACS.

In particular embodiments, an inhibitor directly inhibits expression of or an activity of a target gene, mRNA, or protein, e.g., it may directly bind to the target gene, mRNA or protein. In some embodiments, the inhibitor indirectly inhibits expression of or an activity of a target gene, mRNA, or protein, e.g., it may bind to and inhibit a protein that mediates expression of the target gene, mRNA, or protein (such as a transcription factor), or it may bind to and inhibit expression of an activity of another protein involved in the activity of the target protein (such as another protein present in a complex with the target protein).

In certain embodiments, the inhibitor inhibits SPOP or a protein complex to which SPOP is permanently or transiently associated. In certain embodiments, the protein complex is an SPOP-associated E3 ubiquitin ligase complex. In particular embodiments, the complex comprises Core histone macro-H2A.1 (H2AFY), SPOP, and CUL3; DNA damage-binding protein 1 (DDB1), DNA damage-binding protein 2 (DDB2), Cullin-4A (CUL4A), Cullin-4B (CUL4B), and E3 ubiquitin protein ligase RBX1 (RBX); or Polycomb complex protein BMI-1 (BMI1), SPOP, and CUL3; SPOP, Death domain-associated protein 6 (DAXX), and CUL3; Core histone macro-H2A.1 (H2AFY), SPOP, and CUL3; or BMI1, SPOP, and CUL3. In particular embodiments, the inhibitor inhibits one or more component of any of these complexes. In some embodiments, the inhibitor inhibits expression of SPOP, while in other embodiments, the inhibitor inhibits an activity of SPOP.

In certain embodiments, the inhibitor inhibits CUL3 or a protein complex to which CUL3 is permanently or transiently associated. In certain embodiments, the protein complex is a CUL3-associated E3 ubiquitin ligase complex. In certain embodiments, the CUL3-associated protein complex is a D(4) dopamine receptor (DRD4)-Kelch like protein 12 (KLH12)-CUL3. In certain embodiments, the CUL3-associated protein complex is a coiled coil domain containing protein 22 (CCDC22)-COMM domain containing protein 8 (COMMD8)-CUL3 complex. In certain embodiments, the CUL3-associated protein complex is a Cullin associated NEDD8 dissociated protein (CAND1)-CUL3-E3 ubiquitin protein ligase RBX1 (RBX1). In some embodiments, the complex comprises SPOP, Death domain-associated protein 6 (DAXX), and CUL3; Core histone macro-H2A.1 (H2AFY), SPOP, and CUL3; DNA damage-binding protein 1 (DDB1), DNA damage-binding protein 1 (DDB2), Cullin-4A (CUL4A), Cullin-4B (CUL4B), and E3 ubiquitin-protein ligase RBX1 (RBX1); Polycomb complex protein BMI-1 (BMI1), SPOP, and CUL3; COP9 signalosome complex subunit 1 (CSN1), COP9 signalosome complex subunit 8 (CSN8), Hairy/enhancer-of-split related with YRPW motif protein 1 (HRT1), S-phase kinase-associated protein 1 (SKP1), S-phase kinase-associated protein 2 (SKP2), Cullin-1 (CUL1), Cullin-2 (CUL2), and CUL3; CUL3, Kelch-like protein 3 (KLHL3), and Serine/threonine-protein kinase WNK4 (WNK4); CUL3, KLHL3, and Serine/threonine-protein kinase WNK1 (WNK1); CUL3 and KLHL3. In particular embodiments, the inhibitor inhibits one or more component of any of these complexes. In some embodiments, the inhibitor inhibits expression of CUL3, while in other embodiments, the inhibitor inhibits an activity of CUL3.

In one embodiment, a method of increasing the amount of fetal hemoglobin in a cell, tissue, organ or subject comprises contacting the cell, tissue, organ, or subject with an agent that results in a reduced amount of one or more target genes, mRNAs, or proteins in a cell. In certain embodiments, the agent inhibits the expression or activity of one or more target gene, mRNA, or polypeptide in a cell or tissue. In certain embodiments, the agent causes increased degradation of one or more target gene, mRNA, or polypeptide. In particular embodiments, the cell or tissue is contacted with an amount of the agent effective to reduce the expression or activity of one or more target genes, mRNAs, or polypeptides in the cell or tissue. In certain embodiments, the cell or tissue is contacted with an amount of the agent effective to reduce the amount of active target protein in the cell or tissue. In particular embodiments, the cells are hematopoietic cells, e.g., red blood cells. In certain embodiments, the cells are terminally differentiated, e.g., terminally differentiated red blood cells.

In certain embodiments of any of the methods disclosed herein, the cells comprise one or more mutations associated with a blood cell disorder. e.g., SCD or β-thalassemia. In certain embodiments of any of the methods disclosed herein, the cells have a reduced amount of functionally active HbA as compared to a control cell, e.g., a non-disease cell. In particular embodiments, the cells are associated with a blood cell disorder, e.g., SCD or β-thalassemia. For example, the cells may be derived from or obtained from cells or tissue from a subject diagnosed with the blood cell disorder. In particular embodiments, the methods are practiced on a subject diagnosed with a blood cell disorder, e.g., SCD or β-thalassemia. Methods disclosed herein may be practiced in vitro or in vivo.

In a related aspect, the disclosure includes a method of treating or preventing a blood cell disease or disorder associated with reduced amounts of functionally active HbA (or total HbA) in a subject in need thereof, comprising providing to a subject an agent that inhibits the expression or activity of one or more target protein in the subject, or in certain cells or tissue of the subject, wherein the treatment results in an increased amount of HbF in the subject or one or more cells or tissues of the subject, e.g., hematopoietic cell, e.g., an erythrocyte or red blood cell. In certain embodiments, the agent is present in a pharmaceutical composition. In some embodiments, the subject is provided with one or more (e.g., two, three, or more) agents that inhibits the expression or activity of one or more target protein in the subject, or in certain cells or tissue of the subject. In some embodiments the two or more agents inhibit the same target or target complex disclosed herein, whereas in other embodiments, the two or more agents inhibit different targets or target complexes disclosed herein. In certain embodiments, the cells are terminally differentiated, e.g., terminally differentiated red blood cells. In some embodiments, the agent inhibits the expression or activity of the one or more target protein. In certain embodiments, the agent induces degradation of the one or more target protein. In certain embodiments, the agent inhibits activity of the one or more target protein. In particular embodiments of any of the methods, the inhibitor reduces expression of one or more target genes, mRNAs or proteins in cells or tissue of the subject, e.g., hematopoietic cells, e.g., red blood cells. In particular embodiments, the inhibitor inhibits any of the target genes or proteins identified in Table 1, Table 2, Table 6, Table 7, Table 8, or Table 9, or any component or subunit of any of the complexes identified in Table 3 or Table 4 or pathways identified in Table 5.

In particular embodiments of methods of treatment disclosed herein, the blood disease or disorder is selected from Sickle Cell Disease, β-thalassemia, Beta thalassemia trait or beta thalassemia minor, Thalassemia intermedia, Thalassemia major or Cooley's Anemia.

In particular embodiments of any of the methods described herein, the pharmaceutical composition is provided to the subject parenterally.

Inhibitors and/or other agents and compositions (e.g., inhibitors) described herein can be formulated in any manner suitable for a desired administration route (e.g., parenteral or oral administration). In some embodiments, contacting an agent or composition with a cell and/or tissue is a result of administration of or providing an agent or composition to a subject. In some embodiments, an agent or composition (e.g., an inhibitor) is administered at least 1, 2, 3, 4, 5, 10, 15, 20, or more times. In some embodiments of combination therapies, administration of a first agent or composition is followed by or occurs overlapping with or concurrently with the administration of a second agent or composition. The first and second agent or composition may be the same or they may be different. In some embodiments, the first and second agents or compositions are administered by the same actor and/or in the same geographic location. In some embodiments, the first and second agents or compositions are administered by different actors and/or in different geographical locations. In some embodiments, multiple agents described herein are administered as a single composition.

A wide variety of administration methods may be used in conjunction with the inhibitors according to the methods disclosed herein. For example, inhibitors may be administered or coadministered topically, orally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, intrathecally, transmucosally, pulmonary, or parenterally, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

“Subjects” includes animals (e.g., mammals, swine, fish, birds, insects etc.). In some embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

“Tissue” is an ensemble of similar cells from the same origin that together carry out a specific function.

Methods disclosed herein may be practiced with any agent capable of inhibiting expression or activity of a target gene, mRNA or protein, e.g., an inhibitor of a gene, mRNA or protein, complex or pathway disclosed herein, e.g., in any of Tables 1-9.

In particular embodiments, methods disclosed herein result in a decrease in an expression level or activity of a target gene, mRNA or protein in one or more cells or tissues (e.g., within a subject), e.g., as compared to the expression level or activity in control cells or tissue not contacted with the inhibitor, or a reference level. “Decrease” refers to a decrease of at least 5%, for example, at least 5, 6, 7, 8, 9, 10, 15, 20, 25, 30, 35, 40, 45, 50, 55, 60, 65, 70, 75, 80, 85, 90, 95, 99 or 100%, for example, as compared to the reference level. Decrease also means decreases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a reference or control cells or tissue.

In particular embodiments, methods disclosed herein result in increased amounts of HbF or HBγ in one or more cells or tissues (e.g., within a subject), e.g., as compared to the expression level or activity in control cells or tissue not contacted with the inhibitor, or a reference level. In particular embodiments, methods disclosed herein result in increased expression of a hemoglobin gamma (e.g., HBG1 or HBG2) in one or more cells or tissues (e.g., within a subject), e.g., as compared to the expression level in control cells or tissue not contacted with the inhibitor, or a reference level. “Increase” refers to an increase of at least 5%, for example, at least 5, 6, 7, 8, 9, 10, 15, 20, 25, 30, 35, 40, 45, 50, 55, 60, 65, 70, 75, 80, 85, 90, 95, 99 or 100%, or an at least two-fold, three-fold, give-fold, ten-fold, 20-fold, 50-fold, 100-fold, 500-fold or 1000-fold increase, for example, as compared to the reference level or level in control cells or tissue.

Methods described herein may be practiced using any type of inhibitor that results in a reduced amount or level of a target gene, mRNA or protein, e.g., in a cell or tissue, e.g., a cell or tissue in a subject. In particular embodiments, the inhibitor causes a reduction in active target protein, a reduction in total target protein, a reduction in target mRNA levels, and/or a reduction in target protein activity, e.g., in a cell or tissue contacted with the inhibitor. In certain embodiments, the reduction is at least 5%, at least 10%, at least 20%, at least 30%, at least 40%, at least 50%, at least 60%, at least 70%, at least 80%, or at least 90%, as compared to the level in the same type of cell or tissue not contacted with the inhibitor or a reference level. Methods of measuring total protein or mRNA levels, or activity, in a cell are known in the art. In certain embodiments, the inhibitor inhibits or reduces target protein activity or expression, e.g., mRNA and/or protein expression. In certain embodiments, the inhibitor causes increased degradation of the target protein, resulting in lower amounts of target protein in a cell or tissue.

Inhibitors that may be used to practice the disclosed methods include but are not limited to agents that inhibit or reduce or decrease the expression or activity of a biomolecule, such as but not limited to a target gene, mRNA or protein. In certain embodiments, an inhibitor can cause increased degradation of the biomolecule. In particular embodiments, an inhibitor can inhibit a biomolecule by competitive, uncompetitive, or non-competitive means. Exemplary inhibitors include, but are not limited to, nucleic acids, DNA, RNA, gRNA, shRNA, siRNA, modified mRNA (mRNA), microRNA (miRNA), proteins, protein mimetics, peptides, peptidomimetics, antibodies, small molecules, small organic molecules, inorganic molecules, chemicals, analogs that mimic the binding site of an enzyme, receptor, or other protein, e.g., that is involved in signal transduction, therapeutic agents, pharmaceutical compositions, drugs, and combinations of these. In some embodiments, the inhibitor can be a nucleic acid molecule including, but not limited to, siRNA that reduces the amount of functional protein in a cell. Accordingly, compounds or agents said to be “capable of inhibiting” a particular target protein comprise any type of inhibitor.

In particular embodiments, an inhibitor comprises a nucleic acid that binds to a target gene or mRNA. Accordingly, a nucleic acid inhibitor may comprise a sequence complementary to a target polynucleotide sequence, or a region thereof, or an antisense thereof. In particular embodiments, a nucleic acid inhibitor comprises at least 8, at least 10, at least 12, at least 14, at least 16, at least 20, at least 24, or at least 30 nucleotide sequence corresponding to or complementary to a target polynucleotide sequence or antisense thereof.

In certain embodiments, a nucleic acid inhibitor is an RNA interference or antisense RNA agent or a portion or mimetic thereof, or a morpholino, that decreases the expression of a target gene when administered to a cell. Typically, a nucleic acid inhibitor comprises at least a portion of a target nucleic acid molecule, or an ortholog thereof, or comprises at least a portion of the complementary strand of a target nucleic acid molecule. In some embodiments, expression of a target gene is reduced by at least about 10%, at least about 25%, at least about 50%, at least about 75%, or even 90-100%.

A “complementary” nucleic acid sequence is a nucleic acid sequence capable of hybridizing with another nucleic acid sequence comprised of complementary nucleotide base pairs. By “hybridize” is meant pair to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R (1987) Methods Enzymol. 152:507).

“Antisense” refers to a nucleic acid sequence, regardless of length, that is complementary to a nucleic acid sequence. In certain embodiments, antisense RNA refers to single stranded RNA molecules that can be introduced to an individual cell, tissue, or subject and results in decreased expression of a target gene through mechanisms that do not rely on endogenous gene silencing pathways. An antisense nucleic acid can contain a modified backbone, for example, phosphorothioate, phosphorodithioate, or others known in the art, or may contain non-natural internucleoside linkages. Antisense nucleic acid can comprise, e.g., locked nucleic acids (LNA).

“RNA interference” as used herein refers to the use of agents that decrease the expression of a target gene by degradation of a target mRNA through endogenous gene silencing pathways (e.g., Dicer and RNA-induced silencing complex (RISC)). RNA interference may be accomplished using various agents, including shRNA and siRNA. “Short hair-pin RNA” or “shRNA” refers to a double stranded, artificial RNA molecule with a hairpin turn that can be used to silence target gene expression via RNA interference (RNAi). Expression of shRNA in cells is typically accomplished by delivery of plasmids or through viral or bacterial vectors. shRNA is an advantageous mediator of RNAi in that it has a relatively low rate of degradation and turnover. Small interfering RNA (siRNA) is a class of double-stranded RNA molecules, usually 20-25 base pairs in length, similar to miRNA, and operating within the RNA interference (RNAi) pathway. It interferes with the expression of specific genes with complementary nucleotide sequences by degrading mRNA after transcription, preventing translation. In certain embodiments, an siRNA is 18, 19, 20, 21, 22, 23 or 24 nucleotides in length and has a 2 base overhang at its 3′ end. siRNAs can be introduced to an individual cell and/or culture system and result in the degradation of target mRNA sequences. “Morpholino” as used herein refers to a modified nucleic acid oligomer wherein standard nucleic acid bases are bound to morpholine rings and are linked through phosphorodiamidate linkages. Similar to siRNA and shRNA, morpholinos bind to complementary mRNA sequences. However, morpholinos function through steric-inhibition of mRNA translation and alteration of mRNA splicing rather than targeting complementary mRNA sequences for degradation.

In certain embodiments, a nucleic acid inhibitor is a messenger RNA that may be introduced into a cell, wherein it encodes a polypeptide inhibitor of a target disclosed herein. In particular embodiments, the mRNA is modified, e.g., to increase its stability or reduce its immunogenicity, e.g., by the incorporation of one or more modified nucleosides. Suitable modifications are known in the art.

In certain embodiments, an inhibitor comprises an expression cassette that encodes a polynucleotide or polypeptide inhibitor of a target disclosed herein. In particular embodiments, the expression cassette is present in a gene therapy vector, for example a viral gene therapy vector. A variety of gene therapy vectors, including viral gene therapy vectors are known in the art, including, for example, AAV-based gene therapy vectors.

In some embodiments, an inhibitor is a polypeptide inhibitor. In particular embodiments, a polypeptide inhibitor binds to a target polypeptide, thus inhibiting its activity, e.g., kinase activity. Examples of polypeptide inhibitors include any types of polypeptides (e.g., peptides and proteins), such as antibodies and fragments thereof.

An “antibody” is an immunoglobulin (Ig) molecule capable of specific binding to a target, such as a carbohydrate, polynucleotide, lipid, or polypeptide, through at least one epitope recognition site, located in the variable region of the Ig molecule. As used herein, the term encompasses not only intact polyclonal or monoclonal antibodies, but also fragments thereof, such as dAb, Fab, Fab′, F(ab′)₂, Fv, single chain (scFv), synthetic variants thereof, naturally occurring variants, fusion proteins comprising an antibody portion with an antigen-binding fragment of the required specificity, chimeric antibodies, nanobodies, and any other modified configuration of the immunoglobulin molecule that comprises an antigen-binding site or fragment of the required specificity.

“Fragment” refers to a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. A “functional fragment” of an antibody is a fragment that maintains one or more activities of the antibody, e.g., it binds the same epitope and or possesses a biological activity of the antibody. In particular embodiments, a functional fragment comprises the six CDRs present in the antibody.

In certain embodiments, the inhibitor induces degradation of a target polypeptide. For example, inhibitors include proteolysis targeting chimeras (PROTAC), which induce selective intracellular proteolysis of target proteins. PROTACs include functional domains, which may be covalently linked protein-binding molecules: one is capable of engaging an E3 ubiquitin ligase, and the other binds to the target protein meant for degradation. Recruitment of the E3 ligase to the target protein results in ubiquitination and subsequent degradation of the target protein by the proteasome. In particular embodiments, an inhibitor is a PROTAC that targets any of the targets disclosed herein.

In certain embodiments, an inhibitor is a small molecule inhibitor, or a stereoisomer, enantiomer, diastereomer, isotopically-enriched, pro-drug, or pharmaceutically acceptable salt thereof. In certain embodiments the small molecule inhibitor of a target protein or protein complex that functions to regulate HbF expression targets SPOP. In certain embodiments the small molecule inhibitor of a target protein or protein complex that functions to regulate HbF expression targets CUL3. In certain embodiments, the CUL3 inhibitor is MLN4924 (CAS No: 905579-51-3), suramin (CAS NO: 145-63-1) or DI-591 (CAS No: 2245887-38-9).

In certain embodiments, the inhibitor comprises one or more components of a gene editing system. As used herein, the term “gene editing system” refers to a protein, nucleic acid, or combination thereof that is capable of modifying a target locus of an endogenous DNA sequence when introduced into a cell. Numerous gene editing systems suitable for use in the methods of the present invention are known in the art including, but not limited to, zinc-finger nuclease systems, TALEN systems, and CRISPR/Cas systems.

In some embodiments, the gene editing system used in the methods described herein is a CRISPR (Clustered Regularly Interspaced Short Palindromic Repeats)/Cas (CRISPR Associated) nuclease system, which is an engineered nuclease system based on a bacterial system that can be used for mammalian genome engineering. Generally, the system comprises a CRISPR-associated endonuclease (for example, a Cas endonuclease) and a guide RNA (gRNA). The gRNA is comprised of two parts; a crispr-RNA (crRNA) that is specific for a target genomic DNA sequence, and a trans-activating RNA (tracrRNA) that facilitates endonuclease binding to the DNA at the targeted insertion site. In some embodiments, the crRNA and tracrRNA may be present in the same RNA oligonucleotide, referred to as a single guide-RNA (sgRNA). In some embodiments, the crRNA and tracrRNA may be present as separate RNA oligonucleotides. In such embodiments, the gRNA is comprised of a crRNA oligonucleotide and a tracrRNA oligonucleotide that associate to form a crRNA:tracrRNA duplex. As used herein, the term “guide RNA” or “gRNA” refers to the combination of a tracrRNA and a crRNA, present as either an sgRNA or a crRNA:tracrRNA duplex.

In some embodiments, the CRISPR/Cas systems comprise a Cas protein, a crRNA, and a tracrRNA. In some embodiments, the crRNA and tracrRNA are combined as a duplex RNA molecule to form a gRNA. In some embodiments, the crRNA:tracrRNA duplex is formed in vitro prior to introduction to a cell. In some embodiments, the crRNA and tracrRNA are introduced into a cell as separate RNA molecules and crRNA:tracrRNA duplex is then formed intracellularly. In some embodiments, polynucleotides encoding the crRNA and tracrRNA are provided. In such embodiments, the polynucleotides encoding the crRNA and tracrRNA are introduced into a cell and the crRNA and tracrRNA molecules are then transcribed intracellularly. In some embodiments, the crRNA and tracrRNA are encoded by a single polynucleotides. In some embodiments, the crRNA and tracrRNA are encoded by separate polynucleotides.

In some embodiments, a Cas endonuclease is directed to the target insertion site by the sequence specificity of the crRNA portion of the gRNA, which may include a protospacer motif (PAM) sequence near the target insertion site. A variety of PAM sequences suitable for use with a particular endonuclease (e.g., a Cas9 endonuclease) are known in the art (See e.g., Nat Methods. 2013 November; 10(11): 1116-1121 and Sci Rep. 2014; 4: 5405).

The specificity of a gRNA for a target locus is mediated by the crRNA sequence, which comprises a sequence of about 20 nucleotides that are complementary to the DNA sequence at a target locus, e.g., complementary to a target DNA sequence. In some embodiments, the crRNA sequences used in the methods of the present invention are at least 90% complementary to a DNA sequence of a target locus. In some embodiments, the crRNA sequences used in the methods of the present invention are at least 95%, 96%, 97%, 98%, or 99% complementary to a DNA sequence of a target locus. In some embodiments, the crRNA sequences used in the methods of the present invention are 100% complementary to a DNA sequence of a target locus. In some embodiments, the crRNA sequences described herein are designed to minimize off-target binding using algorithms known in the art (e.g., Cas-OFF finder) to identify target sequences that are unique to a particular target locus or target gene.

In some embodiments, the endonuclease is a Cas protein or ortholog. In some embodiments, the endonuclease is a Cas9 protein. In some embodiments, the Cas9 protein is derived from Streptococcus pyogenes (e.g., SpCas9), Staphylococcus aureus (e.g., SaCas9), or Neisseria meningitides (NmeCas9). In some embodiments, the Cas endonuclease is a Cas9 protein or a Cas9 ortholog and is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the endonuclease is selected from the group consisting of C2C1, C2C3, Cpf1 (also referred to as Cas12a), Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5. Cmr6, Csb1, Csb2, Csb3, Csx17, Csx14, Csx10, Csx16, CsaX, Csx3, Csx1, Csx15, Csf1, Csf2, Csf3, and Csf4. In some embodiments, the Cas9 is a Cas9 nickase mutant. Cas9 nickase mutants comprise only one catalytically active domain (either the HNH domain or the RuvC domain).

In particular aspects, the disclosure includes compositions, e.g., pharmaceutical compositions, comprising an inhibitor of a target disclosed herein, including any of the various classes of inhibitors described herein. The invention encompasses pharmaceutical compositions comprising an inhibitor and a pharmaceutically acceptable carrier, diluent or excipient. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the inhibitor against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of an inhibitor. For example, the inhibitor can be in a crystalline form, in amorphous form, and have any particle size. The particles may be micronized, or may be agglomerated, particulate granules, powders, oils, oily suspensions or any other form of solid or liquid physical form.

When inhibitors exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-a-tocopheryl PEG 1000 succinate (20-50%), using complexation such as HP β-CD and SBE β-CD (10-40%), and using advanced approaches such as micelles, addition of a polymer, nanoparticle suspensions, and liposome formation.

Inhibitors may also be administered or coadministered in slow release dosage forms. Inhibitors may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, syrups, emulsions, oils and the like. For parenteral administration, reconstitution of a lyophilized powder is typically used.

Suitable doses of the inhibitors for use in treating the diseases or disorders described herein can be determined by those skilled in the relevant art. Therapeutic doses are generally identified through a dose ranging study in humans based on preliminary evidence derived from the animal studies. Doses should be sufficient to result in a desired therapeutic benefit without causing unwanted side effects. Mode of administration, dosage forms and suitable pharmaceutical excipients can also be well used and adjusted by those skilled in the art. All changes and modifications are envisioned within the scope of the present patent application.

In certain embodiments, the disclosure includes unit dosage forms of a pharmaceutical composition comprising an agent that inhibits expression or activity of a target polypeptide (or results in reduced levels of a target protein) and a pharmaceutically acceptable carrier, diluent or excipient, wherein the unit dosage form is effective to increase expression of a hemoglobin gamma in one or more tissue in a subject to whom the unit dosage form is administered.

In particular embodiments, the unit dosage forms comprise an effective amount, an effective concentration, and/or an inhibitory concentration, of an inhibitor to treat a blood cell disease or disorder, e.g., one associated with mutant or aberrant hemoglobin beta, including any of the diseases or disorders disclosed herein, e.g., SCD or β-thalassemias.

“Pharmaceutical compositions” include compositions of one or more inhibitors disclosed herein and one or more pharmaceutically acceptable carrier, excipient, or diluent.

“Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

“Effective amount” as used herein refers to an amount of an agent effective in achieving a particular effect, e.g., increasing levels of fetal hemoglobin (or a hemoglobin gamma) in a cell, tissue, organ or subject. In certain embodiments, the increase is at least 10%, at least 20%, at least 30%, at least 40%, at least 50%, at least 60%, or at least 70%, as compared to the amount prior to or without treatment. In the context of therapeutic treatment of a subject, an effective amount may be, e.g., an amount effective or sufficient to reduce one or more disease symptoms in the subject, e.g., a subject with sickle cell disease.

“Effective Concentration” as used herein refers to the minimum concentration (mass/volume) of an agent and/or composition required to result in a particular physiological effect. As used herein, effective concentration typically refers to the concentration of an agent required to increase, activate, and/or enhance a particular physiological effect.

“Inhibitory Concentration” “Inhibitory Concentration” is the minimum concentration (mass/volume) of an agent required to inhibit a particular physiological effect. As used herein, inhibitory concentration typically refers to the concentration of an agent required to decrease, inhibit, and/or repress a particular physiological effect.

In some embodiments, an agent or compound described herein may be administered at a dosage from about 1 mg/kg to about 300 mg/kg. In another embodiment, an agent or compound described herein may be administered at a dosage from about 1 mg/kg to about 20 mg/kg. For example, the agent or compound may be administered to a subject at a dosage of 1, 2, 3, 4, 5, 6, 7, 8, 9, 10, 11, 12, 13, 14, 15, 16, 17, 18, 19 or 20 mg/kg, or within a range between any of the proceeding values, for example, between about 10 mg/kg and about 15 mg/kg, between about 6 mg/kg and about 12 mg/kg, and the like. In another embodiment, an agent or compound described herein is administered at a dosage of ≤15 mg/kg. For example, an agent or compound may be administered at 15 mg/kg per day for 7 days for a total of 105 mg/kg per week. For example, a compound may be administered at 10 mg/kg twice per day for 7 days for a total of 140 mg/kg per week.

In many embodiments, the dosages described herein may refer to a single dosage, a daily dosage, or a weekly dosage. In one embodiment, an agent or compound may be administered once per day. In another embodiment, a compound may be administered twice per day. In some embodiments, an agent or compound may be administered three times per day. In some embodiments, a compound may be four times per day. In some embodiments, an agent or compound described herein may be administered 1, 2, 3, 4, 5, 6, 7, 8, 9, 10, 11, 12, 13, 14, 15, 16, 17, 18, 19, 20, 21, 22, 23, or 24 times per week. In other embodiments, the compound is administered once biweekly.

In some embodiments, an agent or compound described herein may be administered orally. In some embodiments, an agent or compound described herein may be administered orally at a dosage of ≤15 mg/kg once per day.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

The amount and frequency of administration of the compounds of the invention and/or the pharmaceutically acceptable salts thereof will be regulated according to the judgment of the attending clinician considering such factors as age, condition and size of the patient as well as severity of the symptoms being treated.

### EXAMPLES

### Example 1

### Target Identification Methods

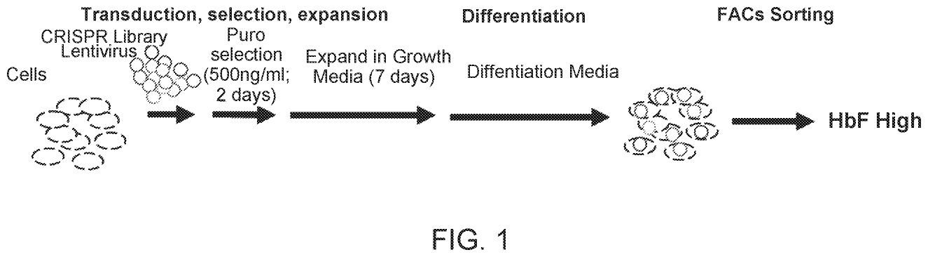

Factors that upregulate HbF protein in the erythroid lineage were identified using a pooled CRISPR screening approach, as diagramed in FIG. 1. HUDEP2 cells, an erythroid progenitor model derived from CD34+ cells isolated from human umbilical cord blood, was used as a cellular model to study HbF reactivation, because the HBB/HBβ globin is the predominant β-like globin expressed.

A pool of CRISPR gRNAs was introduced into proliferating HUDEP2 cells via lentiviral delivery methods at an MOI˜0.1. Depending on the library construction, this was either a one-vector system (vector encoding both the gRNA and Cas9) or a two-vector system (vector encoding the gRNA). For the two-vector system, the lentiviral pool was delivered to HUDEP2 cells constitutively expressing Cas9 protein. One day following lentiviral transduction, the cells were grown in HUDEP2 proliferation media (StemSpan SFEM, StemCell Technologies; 50 ng/ml SCF; 3 IU/ml erythropoietin; 1 uM dexamethasone; 1 ug/ml doxycycline) containing 500 ng/ml puromycin to select for cells that received the CRISPR constructs. Selection in proliferation media+puromycin occurred for 2 days. The selected cells were then expanded for an additional 7 days in proliferation media and then shifted to HUDEP2 differentiation media (Iscove's Modified Dulbecco's Medium; 1% L-glutamine; 2% Penicillin/streptomycin; 330 ug/ml holo-human transferrin; 2 IU/ml heparin; 10 ug/ml recombinant human insulin; 3 IU/ml erythropoietin; 100 ng/ml SCF; 4% fetal calf serum) for 10 days.

An HbF fluorescence-activated cell sorting (FACs) assay (Invitrogen, HFH01) was used to isolate cells with elevated levels of HbF. HbF high cells were selected using HUDEP2 cells transduced with a negative control gRNA (sgGFP) as a gating threshold. Cells were also collected following the 3-day puromycin selection (post-selection sample) and prior to FACs sorting (FACs input sample) and used for downstream analyses to identify hits.

Genomic DNA was isolated from HbF high isolated cells, post-selection sample, and FACs input sample. The gRNA present at in the genomic DNA was amplified using nested PCR amplification. The second round of PCR amplification was performed to also incorporate Illumina sequencing adaptors onto the sample. Illumina sequencing was done to quantify the gRNAs present in each sample. The gRNAs were identified using conserved identifiers and were subsequently mapped to the human reference genome to identify the gRNA target gene to provide the relationship between the target gene and genetic perturbation that led to HbF upregulation.

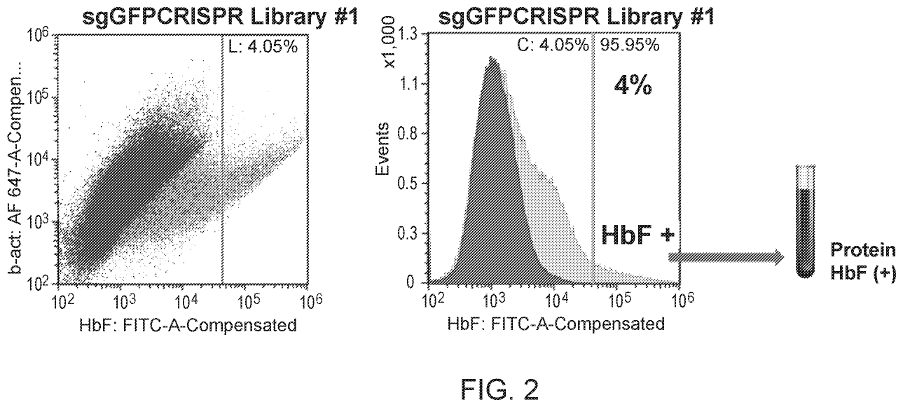

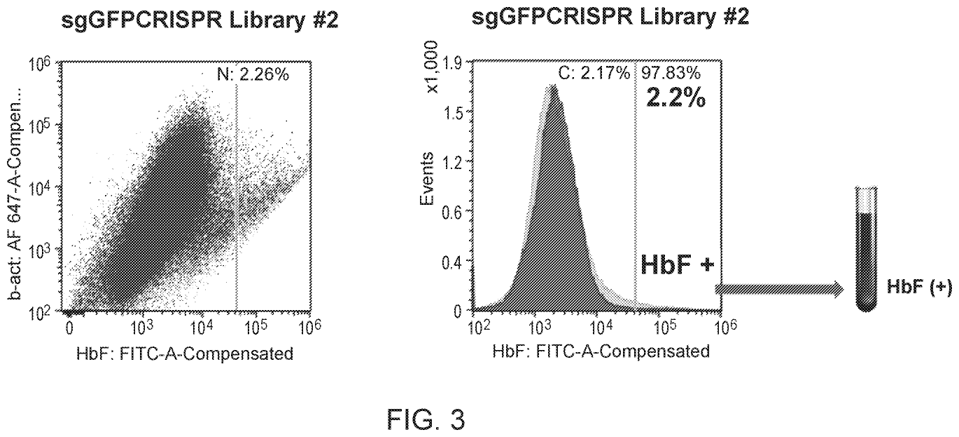

The results of the screens are shown in FIG. 2 (CRISPR Library #1) and FIG. 3 (CRISPR Library #2). For each figure, the left panel plots the level of HbF (X-axis) and β-Actin (Y-axis) for each event, and the line “L” indicates the HbF threshold for HbF high cells. The right panel represents the same data in a one-dimensional plot showing the HbF levels (X-axis) and Events (Y-axis), and the line “C” indicates the HbF threshold for HbF high cells. Any cell above the HbF threshold was collected in the HbF high population. In both FIG. 2 and FIG. 3, the darker shaded cells at the left of each panel are HUDEP2 cells transduced with control sgGFP, and the lighter shaded cells at the right of each panel are HUDEP2 cells transduced with the CRISPR library.

### Example 2

### Computational Methods to Identify GRNAS that Upregulate HBF

Illumina sequencing was used to sequence the libraries of gRNAs in the post-selection samples, FACs input samples, and HbF high samples. Each read was searched for the conserved identifiers either in the 5′ or the 3′ regions, and only reads that contained the conserved identifiers were retained. The 20 bp gRNA sequence between the conserved identifiers was extracted from the retained reads and mapped to the human genome (hg19). A single retained read with a given gRNA represented one count for that gRNA in each sample. The counts were converted to RPM (reads per millions) to normalize for sequencing depth and to enable comparison across different gRNA libraries. The RPM for a gRNA was calculated as follows:

$$g R N {A}_{rpm} = \frac{g R N {A}_{count}}{N} * 1000000$$

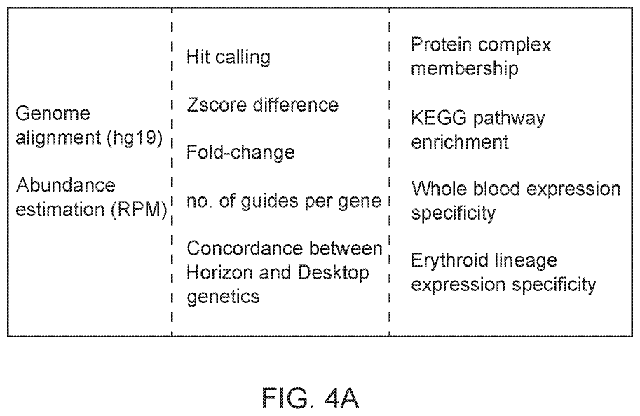

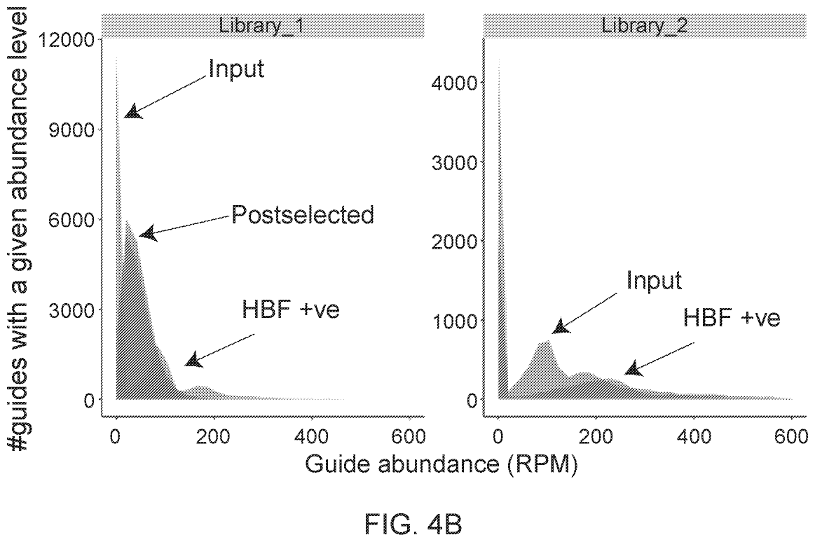

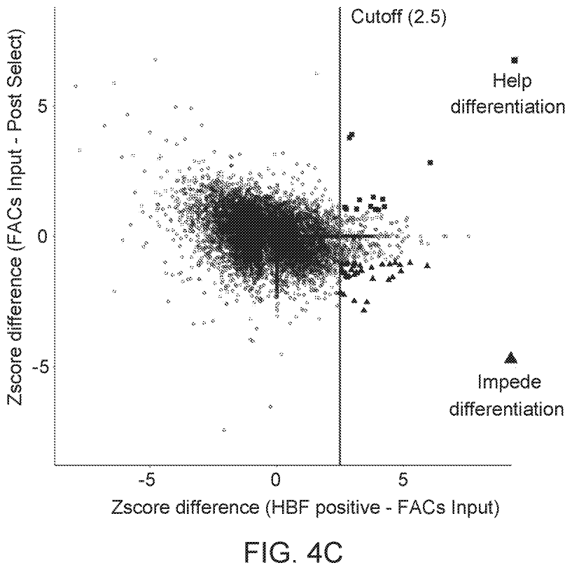

In the above definition, N is the total number of reads in the library. Four different statistical methods were used to identify hits among the HbF high sample. The bioinformatics analysis performed using method 2 described below is summarized in FIG. 4A. FIG. 4B shows the distribution of guide abundance in different samples from two different screening libraries (Library #1 and Library #2), and FIG. 4C shows Z-score differences across samples for Library #1.

### Method 1: A Z Score Based Approach in HbF High Samples:

In this approach, a Z score was calculated based on the distribution of gRNAᵣₚₘ values in the HbF sample. More formally, the following formula was used to calculate the Z score

$$gRN {A}_{HbF +} = \frac{gRN {A}_{rpm , Hbf +} - {μ}_{Hbf +}}{{σ}_{H b f +}}$$

In the above equation gRNA_{HbF+} is the Z score in HbF+ samples, gRNA_{rpm,Hbf+} is the abundance, μ_{Hbf+}, and σ_{Hbf+} are the mean and standard-deviation of gRNA_{rpm,Hbf+} in HbF+ samples. Similarly Z scores were calculated in the Input (gRNAᵢₙₚᵤₜ) and post-selected (gRNA_{post-selected}) samples for all guides. gRNAs that led to a negative impact on cell health or proliferation were identified by performing a gRNA dropout analysis. More formally, all guides with |gRNAᵢₙₚᵤₜ−gRNA_{post-selected}|≥1 were removed in this dropout analysis. All the remaining gRNAs with gRNA_{HbF+}>3 were considered as enriched in HbF+ samples. Using this approach, a total of 174 hits were identified that contained at least one enriched gRNA.

### Method 2: A Z Score Difference Based Approach in HbF High and FACs Input:

In this approach, the same dropout analysis (as performed in method 1) was performed. All gRNAs with gRNA_{HbF+}−gRNAᵢₙₚᵤₜ>2.5 were considered as enriched in HbF+ samples. Using this approach, a total of 307 hits were identified that contained at least one enriched gRNA. These are provided in Table 1.

TABLE I
List of targets that upregulate HbF protein

| Gene | | |
|---|---|---|
| Name | Uniprot ID | Description |
| CSNK1G2 | P78368 | casein kinase 1 gamma 2 |
| HIST1H2AA | Q96QV6 | histone cluster 1 H2A family member a |
| CDYL2 | Q8N8U2 | chromodomain Y like 2 |
| CAT | P04040 | catalase |
| KDM5A | P29375 | lysine demethylase 5A |
| PRKDC | P78527 | protein kinase, DNA-activated, catalytic polypeptide |
| SIM1 | P81133 | single-minded family bHLH transcription factor 1 |
| CCDC77 | Q9BR77 | coiled-coil domain containing 77 |
| SMYD1 | Q8NB12 | SET and MYND domain containing 1 |
| ASS1 | Q5T6L4 | argininosuccinate synthase 1 |
| CROT | Q9UKG9 | carnitine O-octanoyltransferase |
| CUL3 | Q13618 | cullin 3 |
| L3MBIL3 | Q96JM7 | L3MBTL3, histone methyl-lysine binding protein |
| GDNF | P39905 | glial cell derived neurotrophic factor |
| SAP130 | Q9H0E3 | Sin3A associated protein 130 |
| CDKN1C | P49918 | cyclin dependent kinase inhibitor 1C |
| ATP5F1C | P36542 | ATP synthase Fl subunit gamma |
| EID1 | Q9Y632 | EP300 interacting inhibitor of differentiation 1 |
| DNAJC1 | Q96KC8 | Dnaj heat shock protein family (Hsp40) member C1 |
| EXOSC1 | Q9Y3B2 | exosome component 1 |
| PGAM4 | Q8N0Y7 | phosphoglycerate mutase family member 4 |
| CHD1 | O14646 | chromodomain helicase DNA binding protein 1 |
| TSHZ3 | Q63HK5 | teashirt zinc finger homeobox 3 |
| TADA3 | O75528 | transcriptional adaptor 3 |
| HIBADH | P31937 | 3-hydroxyisobutyrate dehydrogenase |
| WRB | O00258 | tryptophan rich basic protein |
| IKZF2 | Q9UKS7 | IKAROS family zinc finger 2 |
| TK2 | O00142 | thymidine kinase 2, mitochondrial |
| LDHB | Q5U077 | lactate dehydrogenase B |
| SIRT3 | Q9NTG7 | sirtuin 3 |
| HIST1H1T | P22492 | histone cluster 1 H1 family member t |
| ROCK2 | Q14DU5 | Rho associated coiled-coil containing protein kinase 2 |
| DIP2C | Q9Y2E4 | disco interacting protein 2 hornolog C |
| NAP1L4 | Q99733 | nucleosome assembly protein 1 like 4 |
| PRKD3 | O94806 | protein kinase D3 |
| KIDM3B | Q7L3C6 | lysine demethylase 33 |
| C22orf39 | Q6P5X5 | chromosome 22 open reading frame 39 |
| ADCY8 | P40145 | adenylate cyclase 8 |
| HIRA | P54198 | histone cell cycle regulator |
| USP3 | Q916I4 | ubiquitin specific peptidase 3 |
| MSL3 | Q8N5Y2 | MSL complex subunit 3 |
| HIST1H1B | P16401 | histone cluster 1 H1 family member b |
| HMG20B | Q9P0W2 | high mobility group 203 |
| BMX | P51813 | BMX non-receptor tyrosine kinase |
| KDM4E | B2RXH2 | lysine demethylase 4E |
| EEF2K | O00418 | eukaryotic elongation factor 2 kinase |
| PYGB | P11216 | glycogen phosphorylase B |
| MTA2 | O94776 | metastasis associated 1 family member 2 |
| SLC2A8 | Q9NY64 | solute carrier family 2 member 8 |
| NADK | O95544 | NAD kinase |
| PRMT1 | H7C2I1 | protein arginine methyltransferase 1 |
| HIST1H3D | P68431 | histone cluster 1 H3 family member d |
| PRKAR2B | P31323 | protein kinase cAMP-dependent type II regulatory subunit beta |
| ROS1 | P08922 | ROS proto-oncogene 1, receptor tyrosine kinase |
| ITPKC | Q96DU7 | inositol-trisphosphate 3-kinase C |
| AK1 | Q6FGX9 | adenylate kinase 1 |
| SSRP1 | Q08945 | structure specific recognition protein 1 |
| PADI4 | Q9UM07 | peptidyl arginine deiminase 4 |
| RB1 | Q92728 | RB transcriptional corepressor 1 |
| RRM2 | P31350 | ribonucleotide reductase regulatory subunit M2 |
| CDK10 | Q9UHL7 | cyclin dependent kinase 10 |
| G6PC3 | Q9BUM1 | glucose-6-phosphatase catalytic subunit 3 |
| GRK5 | P34947 | G protein-coupled receptor kinase 5 |
| BARD1 | Q99728 | BRCA1 associated RING domain 1 |
| MYLK2 | Q9H1R3 | myosin light chain kinase 2 |
| YWHAE | V9HW98 | tyrosine 3-monooxygenase/tryptophan 5-monooxygenase |
| | | activation protein epsilon |
| GCDH | Q92947 | glutaryl-CoA dehydrogenase |
| TPI1 | V9HWK1 | triosephosphate isornerase 1 |
| PDK1 | Q15118 | pyruvate dehydrogenase kinase 1 |
| DCK | P27707 | deoxycytidine kinase |
| UBR2 | Q8IWV8 | ubiquitin protein ligase E3 component n-recognin 2 |
| IDH3G | P51553 | isocitrate dehydrogenase 3 (NADH) gamma |
| SLC13A2 | Q13183 | solute carrier family 13 member 2 |
| TOP2A | P11388 | DNA topoisomerase II alpha |
| PDP1 | Q9P0J1 | pyruvate dehyrogenase phosphatase catalytic subunit 1 |
| PRPS1 | P60891 | phosphoribosyl pyrophosphate synthetase 1 |
| PHF7 | Q9BWX1 | PHD finger protein 7 |
| FBL | P22087 | fibrillarin |
| LDHAL6A | Q6ZMR3 | lactate dehydrogenase A like 6A |
| TEX14 | Q81W66 | testis expressed 14, intercellular bridge forming factor |
| PCCA | P05165 | propionyl-CoA carboxylase alpha subunit |
| PDK3 | Q15120 | pyruvate dehydrogenase kinase 3 |
| FADS1 | A0A0A0MR51 | fatty acid desaturase 1 |
| ATXN7L3 | Q14CW9 | ataxin 7 like 3 |
| RPS6KA4 | O75676 | ribosomal protein S6 kinase A4 |
| PC | P11498 | pyruvate carboxylase |
| GPX5 | V9HWN8 | glutathione peroxidase 5 |
| GPX6 | P59796 | glutathione peroxidase 6 |
| ARID4A | P29374 | AT-rich interaction domain 4A |
| USP16 | Q9Y515 | ubiquitin specific peptidase 16 |
| ITGB3 | Q16157 | integrin subunit beta 3 |
| RMI1 | Q9H9A7 | RecQ mediated genome instability 1 |
| SLC27A5 | Q9Y2P5 | solute carrier family 27 member 5 |
| PANK4 | Q9NVE7 | pantothenate kinase 4 |
| GALM | Q96C23 | galactose mutarotase |
| SRC | P12931 | SRC proto-oncogene, non-receptor tyrosine kinase |
| ADCY1 | Q08828 | adenylate cyclase 1 |
| RNF17 | Q9BXT8 | ring finger protein 17 |
| PFKFB4 | Q66535 | 6-phosphofructo-2-kinase/fructose-2,6-biphosphatase 4 |
| COTL1 | Q14019 | coactosin like F-actin binding protein 1 |
| PHIP | Q8WWQ0 | pleckstrin homology domain interacting protein |
| BRWD1 | Q9NSI6 | brornodornain and WD repeat domain containing 1 |
| MBD3 | O95983 | methyl-CpG binding domain protein 3 |
| GCK | Q53Y25 | glucokinase |
| TYRO3 | Q06418 | TYRO3 protein tyrosine kinase |
| BCAT1 | P54687 | branched chain amino acid transaminasel |
| SMARCC1 | Q92922 | SWI/SNF related, matrix associated, actin dependent regulator of |
| | | chromatin subfamily c member 1 |
| CBX4 | O00257 | chromobox 4 |
| ULK4 | Q96C45 | unc-51 like kinase 4 |
| GCLC | Q14TF0 | glutamate-cysteine ligase catalytic subunit |
| LYN | P07948 | LYN proto-oncogene, Src family tyrosine kinase |
| EZH2 | S453R8 | enhancer of zeste 2 polycomb repressive complex 2 subunit |
| EXR2 | P51116 | FMK autosomal homolog 2 |
| MGAM | O43451 | maltase-glucoamylase |
| CDK5R1 | Q15078 | cyclin dependent kinase 5 regulatory subunit 1 |
| PHF13 | Q86YI8 | PHD finger protein 13 |
| MAPK13 | O15264 | mitogen-activated protein kinase 13 |
| DGUOK | Q16854 | deoxyguanosine kinase |
| TNK1 | Q13470 | tyrosine kinase non receptor 1 |
| TET3 | O43151 | tet methylcytosine dioxygenase 3 |
| NAP1L2 | Q9ULW6 | nucleosome assembly protein 1 like 2 |
| SMARCB1 | Q12824 | SWI/SNF related, matrix associated, actin dependent regulator of |
| | | chromatin, subfamily b, member 1 |
| L3MBTL1 | Q9Y468 | L3MBTL1, historic methyl-lysine binding protein |
| CAMK2G | Q8WU40 | calcium/calmodulin dependent protein kinase II gamma |
| SETD1A | O15047 | SET domain containing 1A |
| PHF3 | Q92576 | PHD finger protein 3 |
| CUL4B | Q13620 | cuilin 43 |
| EPHA5 | P54756 | EPH receptor A5 |
| BDH2 | Q9BUT1 | 3-hydroxybutyrate dehydrogenase 2 |
| FLT4 | P35916 | fms related tyrosine kinase 4 |
| CAMK2B | Q13554 | calcium/calmodulin dependent protein kinase II beta |
| PHF12 | Q96QT6 | PHD finger protein 12 |
| CCDC169 | A6NNP5 | coiled-coil domain containing 169 |
| AMT | P48728 | aminomethyltransferase |
| TRIB3 | Q963U7 | tribbles pseudokinase 3 |
| AUH | Q13825 | AU RNA binding methylglutaconyl-CoA hydratase |
| NOC2L | Q9Y3T9 | NOC2 like nucleolar associated transcriptional repressor |
| UQCRC1 | P31930 | ubiquinol-cytochrome c reductase core protein 1 |
| SIK36 | Q9N3P7 | serine/threonine kinase 36 |
| HDGF | P51858 | heparin binding growth factor |
| INSRR | P14616 | insulin receptor related receptor |
| MCAT | Q8IVS2 | malonyl-CoA-acyl carrier protein transacylase |
| AURKA | O14965 | aurora kinase A |
| USP46 | P62068 | ubiquitin specific peptidase 46 |
| FGFR1 | P11362 | fibroblast growth factor receptor 1 |
| RLIM | Q9NVW2 | ring finger protein, LIM ciomain interacting |
| MYBBP1A | Q9BQGO | MYB binding protein 1a |
| MAPK4 | P31152 | mitogen-activated protein kinase 4 |
| RPS6KA3 | P51812 | ribosomal protein S6 kinase A3 |
| ULK2 | Q8IYT8 | unc-51 like autophagy activating kinase 2 |
| NPM2 | Q86SE8 | nucleophosmininucleoplasmin 2 |
| CDKN1B | Q6I9V6 | cyclin dependent kinase inhibitor 1B |
| EHHADH | Q08426 | enoyl-CoA hydratase and 3-hydroxyacyl CoA dehydrogenase |
| ADCK2 | Q7Z695 | aarF domain containing kinase 2 |
| PRMI2 | P55345 | protein arginine methyltransferase 2 |
| PRPF4B | Q13523 | pre-mRNA processing factor 4B |
| AMD1 | Q5VXN5 | adenosylmethionine decarboxylase 1 |
| ECI2 | O75521 | enoyl-CoA delta isomerase 2 |
| SBK1 | Q52WX2 | SH3 domain binding kinase 1 |
| MAP4K4 | O95819 | mitogen-activated protein kinase kinase kinase kinase 4 |
| HIF1AN | Q9NWT6 | hypoxia inducible factorl alpha subunit inhibitor |
| ALDOA | V9HWN7 | aldolase, fructose-bisphosphate A |
| INO80C | Q6P198 | INO80 complex subunit C |
| SIRT7 | Q9NRC8 | sirtuin 7 |
| AIRE | O43918 | autoimmune regulator |
| SRSF3 | P84103 | serine and arginine rich splicing factor 3 |
| BDH1 | Q02338 | 3-hydroxybutyrate dehydrogenase 1 |
| SETD4 | Q9NVD3 | SET domain containing 4 |
| CDKN1A | P38936 | cyclin dependent kinase inhibitor 1A |
| TAF6L | Q9Y619 | TATA-box binding protein associated factor 6 like |
| ADCY9 | O60503 | adenylate cyclase 9 |
| PHF1 | O43189 | PHD finger protein 1 |
| BEX3 | Q00994 | brain expressed X-linked 3 |
| USP21 | Q9UK80 | ubiquitin specific peptidase 21 |
| SMYD2 | Q9NRG4 | SET and MYND domain containing 2 |
| G6PC | P35575 | glucose-6-phosphatase catalytic subunit |
| PHC2 | Q8IXKO | polyhorneotic homolog 2 |
| FBX043 | Q4G163 | F-box protein 43 |
| CDK8 | P49336 | cyclin dependent kinase 8 |
| HMGCS1 | Q01581 | 3-hydroxy-3-methylglutaryl-CoA synthase 1 |
| SPEN | Q96158 | spen family transcriptional repressor |
| ELP2 | Q6IA86 | elongator acetyltransferase complex subunit 2 |
| FFAR2 | O15552 | free fatty acid receptor 2 |
| RNF8 | O76064 | ring finger protein 8 |
| ZNF266 | Q14584 | zinc finger protein 266 |
| MST1 | G3XAK1 | macrophage stimulating 1 |
| PHF19 | Q5T6S3 | PHD finger protein 19 |
| IGF1R | P08069 | insulin like growth factor 1 receptor |
| MARK1 | Q9P0L2 | microtubule affinity regulating kinase I |
| FES | P07332 | FES proto-oncogene, tyrosine kinase |
| SMARCA1 | P28370 | SWI/SNF related, matrix associated, actin dependent regulator of |
| | | chromatin, subfamily a, member 1 |
| ADCY7 | P51828 | adenylate cyclase 7 |
| PGLS | O95336 | 6-phosphogluconolactonase |
| SPOP | O43791 | speckle type BTB/POZ protein |
| ATF7IP | Q6VMQ6 | activating transcription factor 7 interacting protein |
| KDMSD | Q9BY66 | lysine demethylase SD |
| TADA1 | Q96BN2 | transcriptional adaptor 1 |
| IKZF3 | Q9UKT9 | IKAROS family zinc finger 3 |
| IKZF1 | R9R4D9 | IKAROS family zinc finger 1 |
| MGST2 | Q99735 | microsomal glutathione S-transferase 2 |
| CALM1 | Q96HY3 | calmodulin I |
| TPK1 | Q9H354 | thiamin pyrophosphokinase 1 |
| MYO3A | Q8NEV4 | myosin IIIA |
| SIN3A | Q96ST3 | SIN3 transcription regulator family member A |
| AOX1 | Q06278 | aldehyde oxidase 1 |
| NME7 | Q9Y5B8 | NME/NM23 family member 7 |
| PAR P1 | P09874 | poly(ADP-ribose) polymerase 1 |
| SCYL3 | Q8IZE3 | SCY1 like pseudokinase 3 |
| PASK | Q96RG2 | PAS domain containing serine/threonine kinase |
| MEAF6 | Q9HAF1 | MYST/Esa1 associated factor 6 |
| STK17A | Q9UEE5 | serine/threonine kinase 17a |
| ACADVL | P49748 | acyl-CoA dehydrogenase very long chain |
| PKN3 | Q6P5Z2 | protein kinase N3 |
| ACACB | O00763 | acetyl-CoA carboxylase beta |
| ZCWPW2 | Q504Y3 | zinc finger CW-type and PWWP domain containing 2 |
| FUK | Q8NOW3 | fucokinase |
| ADH5 | Q6IRT1 | alcohol dehydrogenase 5 (class III), chi polypeptide |
| CIR1 | Q86X95 | corepressor interacting with RBPJ, 1 |
| GOLGA5 | QBTBA6 | golgin AS |
| APOBEC3G | Q9HC16 | apolipoprotein B mRNA editing enzyme catalytic subunit 3G |
| PRDM11 | Q9NQV5 | PR/SET domain 11 |
| HLCS | P50747 | holocarboxylase synthetase |
| OBSCN | Q5VST9 | obscurin, cytoskeletal calmodulin and titin-interacting RhoGEF |
| APOBEC3H | M4W6S4 | apolipoprotein B mRNA editing enzyme catalytic subunit 3H |
| ADH4 | P08319 | alcohol dehydrogenase 4 (class II), pi polypeptide |
| HIST3H3 | Q16695 | histone cluster 3 H3 |
| HMG20A | Q9NP66 | high mobility group 20A |
| FAM208A | Q9UK61 | family with sequence similarity 208 member A |
| SRP72 | V9HWK0 | signal recognition particle 72 |
| TAF5L | O75529 | TATA-box binding protein associated factor 5 like |
| MVK | Q03426 | mevalonate kinase |
| HIST4H4 | P62805 | histone cluster 4 H4 |
| SRPK2 | P78362 | SRSF protein kinase 2 |
| RPL27 | P61353 | ribosomal protein L27 |
| FLT3 | P36888 | fms related tyrosine kinase 3 |
| CS | O75390 | citrate synthase |
| GUCY2D | Q02846 | guanylate cyclase 2D, retinal |
| CPT1B | Q92523 | carnitine palmitayltransferase IB |
| EGFR | Q504U8 | epidermal growth factor receptor |
| MAST3 | O60307 | microtubule associated serine/threonine kinase 3 |
| MAGI2 | Q86UL8 | membrane associated guanylate kinase, WW and PDZ domain |
| | | containing 2 |
| SLC5A1 | P13866 | solute carrier family 5 member 1 |
| IRAK4 | Q9NWZ3 | interleukin I receptor associated kinase 4 |
| NAP1L1 | P55209 | nucleosome assembly protein 1 like 1 |
| MAGI1 | Q96QZ7 | membrane associated guanylate kinase, WW and PDZ domain |
| | | containing 1 |
| GAPDH | V9HVZ4 | glyceraldehyde-3-phosphate dehydrogenase |
| PRDM6 | Q9NQX0 | PR/SET domain 6 |
| PARP2 | Q9UGN5 | poly(ADP-ribose) polymerase 2 |
| MYBL1 | P10243 | MYB proto-oncogene like 1 |
| NASP | Q5T626 | nuclear autoantigenic sperm protein |
| CTBPI | X5D8Y5 | C-terminal binding protein 1 |
| NFYC | Q13952 | nuclear transcription factor Y subunit gamma |
| PIK3C2A | O00443 | phosphatidylinositol-4-phosphate 3-kinase catalytic subunit type |
| | | 2 alpha |
| PRKAA2 | P54646 | protein kinase AMP-activated catalytic subunit alpha 2 |
| CUL4A | Q13619 | cullin 4A |
| SLC2A5 | P22732 | solute carrier family 2 member 5 |
| TAF10 | Q12962 | TATA-box binding protein associated factor 10 |
| RRP8 | O43159 | ribosomal RNA processing 8 |
| DTYMK | Q6FGU2 | deoxythymidylate kinase |
| YWHAZ | P63104 | tyrosine 3-monooxygenase/tryptophan 5-monooxygenase |
| | | activation protein zeta |
| SUCLG1 | P53597 | succinate-CoA ligase alpha subunit |
| KMT2C | Q8NEZ4 | lysine methyltransferase 2C |
| TTBK2 | C18IWY7 | tau tubulin kinase 2 |
| SIRT2 | Q81X16 | sirtuin 2 |
| DAB2IP | Q5VWQ8 | DAB2 interacting protein |
| CAMK1G | Q96NX5 | calcium/calmodulin dependent protein kinase IG |
| PAK5 | Q9P286 | p21 (RAC1) activated kinase 5 |
| TXNDC12 | O95881 | thioredoxin domain containing 12 |
| TESK2 | Q96S53 | testis-specific kinase 2 |
| MAPK11 | Q15759 | mitogen-activated protein kinase 11 |
| MAGI3 | A0A024R0H3 | membrane associated guanylate kinase, WW and PDZ domain |
| | | containing 3 |
| MAP2K5 | Q13163 | mitogen-activated protein kinase kinase 5 |
| BPGM | P07738 | bisphosphoglycerate mutase |
| PIK3CB | Q68DL0 | phosphatidylinosito1-4,5-bisphosphate 3-kinase catalytic subunit beta |
| YEATS2 | Q9ULM3 | YEATS domain containing 2 |
| EXOSC9 | Q06265 | exosorne component 9 |
| NEK1 | Q96PY6 | NIMA related kinase 1 |
| MYLK | Q15746 | myosin light chain kinase |
| CYP4A11 | Q02928 | cytochrome P450 family 4 subfamily A member 11 |
| AKT1 | P31749 | AKT serine/threonine kinase 1 |
| SETDB1 | Q15047 | SET domain bifurcated 1 |
| CDK17 | Q00537 | cyclin dependent kinase 17 |
| HLTF | Q14527 | helicase like transcription factor |
| IDH2 | P48735 | isocitrate dehydrogenase (NADP(+)) 2, mitochondrial |
| LRWD1 | Q9UFC0 | leucine rich repeats and WD repeat domain containing 1 |
| CPT2 | P23786 | carnitine palmitoyltransferase 2 |
| PRKACB | P22694 | protein kinase cAMP-activated catalytic subunit beta |
| ZNF687 | Q8N1G0 | zinc finger protein 687 |
| UBE2H | P62256 | ubiquitin conjugating enzyme E2 H |
| HMGN2 | P05204 | high mobility group nucleosornal binding domain 2 |
| ACAD10 | Q6JQN1 | acyl-CoA dehydrogenase family member 10 |
| TBK1 | Q9UHD2 | TANK binding kinase 1 |
| PRDM8 | CI9NOV8 | PR/SET domain 8 |
| ERB33 | P21860 | erb-b2 receptor tyrosine kinase 3 |
| ARID1A | O14407 | AT-rich interaction domain 1A |
| DNMT1 | P26358 | DNA methyltransferase 1 |
| CAMK2D | Q13557 | calcium/calmodulin dependent protein kinase \|\| delta |
| EPHB3 | P54753 | EPH receptor 63 |
| MBD4 | O95243 | methyl-CpG binding domain 4, DNA glycosylase |
| PRMT8 | Q9NR22 | protein arginine methyltransferase 8 |
| MTF2 | Q96G26 | metal response element binding transcription factor 2 |
| GLYR1 | Q49A26 | glyoxylate reductase 1 homolog |
| FRK | P42685 | fyn related Src family tyrosine kinase |
| ACAD8 | Q9UKU7 | acyl-CoA dehydrogenase family member 8 |
| RIMKLB | Q9ULI2 | ribosomal modification protein rirriK like family member B |
| ACADS | P16219 | acyl-CoA dehydrogenase short chain |
| SMARCAL1 | Q9NZC9 | SWI/SNF related, matrix associated, actin dependent regulator of |
| | | chromatin, subfamily a like 1 |

### Method 3: A Fold-Change Based Approach in HbF High and FACs Input:

In this approach, the dropout and the hit calling was performed using fold-changes of RPM values. More formally

$$\vert log ( \frac{gRN {A}_{rpm , input}}{gRN {A}_{rpm , post - selected}} ) \vert \geq 2$$

was used as the criteria for gRNA dropout. After the dropout filtration, all the remaining gRNAs with

$$\vert log ( \frac{gRN {A}_{rpm , Hbg +}}{gRN {A}_{rpm , input}} ) \vert \geq 3$$

were considered as enriched in HbF+ samples. Using this approach, a total of 314 hits were identified that contained at least one enriched gRNA.
Number of gRNA Hits Per Gene:

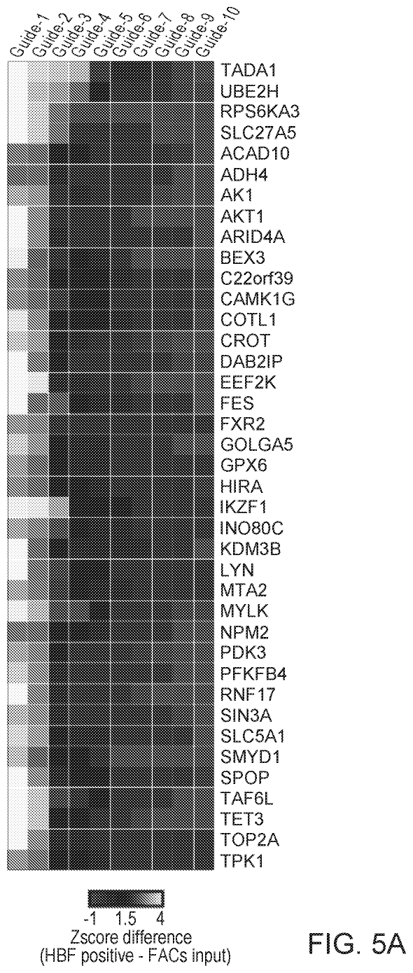

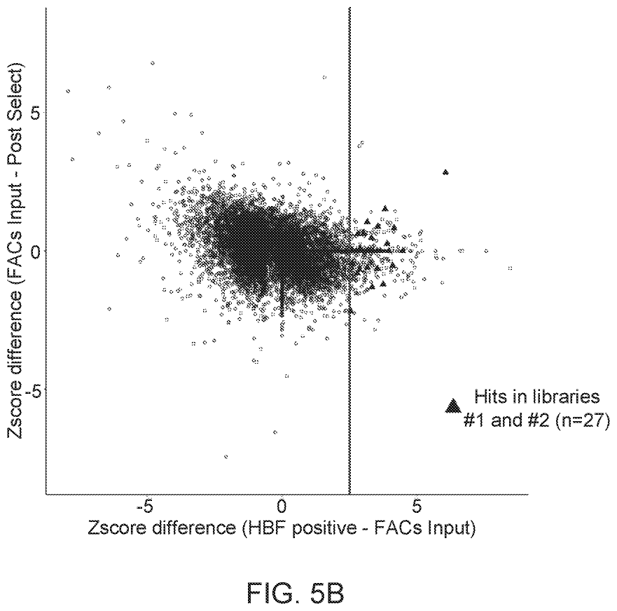

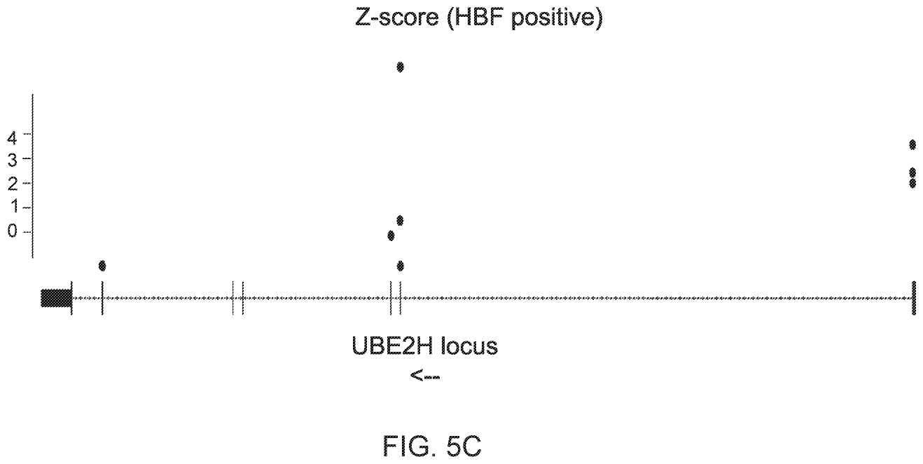

In this approach, method 2 was used to identify enriched gRNAs. Genes with at least two enriched gRNAs were considered as hits. Using this approach 39 hits were identified. These are listed in FIG. 5A. A list of hits and associated gRNAs is summarized in Table 2.

TABLE 2
List of illustrative gRNAs for targets
that upregulate HbF

| | | Seq |
|---|---|---|
| | Guide | ID |
| Gene | Sequence | No. |
| MTA2 | GCAAAGGAACGGCTACGACC | 5 |
| AK1 | TTGAAACGTGGAGAGACCAG | 6 |
| AK1 | GCTGTCGGAAATCATGGAGA | 7 |
| AKT1 | GCAGGATGTGGACCAACGTG | 8 |
| ARID4A | TGAGCCTGCCTACCTGACAG | 9 |
| UBE2H | CAGTCCGGGCAAGAGGCGGA | 10 |
| BEX3 | GACTTGCCCCTAATTTTCGA | 11 |
| COTL1 | TGCACTGCTGGATGAAGTGC | 12 |
| GROT | GGAGCGAACTCGATGGGCTA | 13 |
| CROT | ACTACTGGCCTCCAAAGGAA | 14 |
| DAB2IP | TGTGTGAGCTCAGGGAGCTG | 15 |
| ADH4 | GTTTGTGAAGGCTAAAGCCC | 16 |
| EEF2K | GGGGACAGCGACGATGAGGA | 17 |
| EEF2K | ATGGTGCGCTACCACGAGGG | 18 |
| FES | GGAGGGCATGAGAAAGTGGA | 19 |
| FXR2 | GGTTTAGTGCGTTCCAGGGG | 20 |
| CAMK1G | GCTGCATGACCAGGTAGTAG | 21 |
| GOLGA5 | GGAGAGCTATAAACAGATGC | 22 |
| GOLGA5 | TCTTTTGGGAGCCAAACCCA | 23 |
| GPX6 | TCTCAAAGAGCTGGAAACTG | 24 |
| SLC5A1 | GAGGAGGGAGATGACCACGA | 25 |
| IKZF1 | ATAAGGTCTCACCTGAAACT | 26 |
| IKZF1 | AGGCCCCGCACTGATTGCAC | 27 |
| RNF17 | AATAAGGCTCCAAAAGACCA | 28 |
| INO80C | GCAATGCCCTTTCAGAAGCG | 29 |
| KDM3B | GTAGACAGTAATGGGAGCGA | 30 |
| TET3 | GAGGCTGGGAACAACAGCAG | 31 |
| LYN | GTTTGGCCACATAGTTGCTG | 32 |
| MTA2 | GGTGCTGTGTCGGGATGAGA | 33 |
| MYLK | TCGCGATTTAGAAGTTGTGG | 34 |
| MYLK | AATGAGCTCTGCTGTGCAGG | 35 |
| TAF6L | GAACCTGGCACCTCAAGGAT | 36 |
| PDK3 | TAAGAGCCCTGAGGATCCAT | 37 |
| PFKFB4 | GGGTTCTGTGTCAATTCCCG | 38 |
| UBE2H | GCCCGGACTGGGAGATGAGA | 39 |
| SLC27A5 | GCCATACCTCCCCTACACCA | 40 |
| RNF17 | GCCTTGATGAAGCACTGCAG | 41 |
| RPS6KA3 | CCCGTGGCAGAAGATGGCTG | 42 |
| RPS6KA3 | ACATCTCTTGCAAACAGAGT | 43 |
| SIN3A | GGTGTGTGAGGCTGGACCGG | 44 |
| SLC27A5 | GGGGCTGCTGCTGACCAAGG | 45 |
| SLC27A5 | GCTCAGCACAGAGTGCGCCA | 46 |
| SLC5A1 | CACCATGGACATCTACGCCA | 47 |
| SMYD1 | TGAGCGGGCTTATTCCGCAG | 48 |
| SPOP | GTTTGTGCAAGGCAAAGACT | 49 |
| SPOP | TAACTTTAGCTTTTGCCGGG | 91 |
| SPOP | CGGGCATATAGGTTTGTGCA | 92 |
| SPOP | GTTTGCGAGTAAACCCCAAA | 93 |
| TADA1 | AGTGGGAAGCATCATTGTGT | 50 |
| TADA1 | ACTGGGCTAACCTAAAGCTG | 51 |
| TADA1 | GACCTTTGTGAGCGAGCTGG | 52 |
| TADA1 | AGATCGTACATGTTCACCGG | 53 |
| TAF6L | GGACACTGCCCACCAGACAG | 54 |
| TET3 | GGCACCTCTGAGCTGAGGAG | 55 |
| TOP2A | GAAGAGAGGGCCAGTTGTGA | 56 |
| UBE2H | GAGGCGGATGGACACGGACG | 57 |
| UBE2H | CAAATTCATTAAGTCCTCCC | 58 |
| ACAD10 | GAGGTCTTCGATCAGTGGGG | 59 |
| ACAD10 | GCTGGGAATCCCTGCTGCAG | 60 |
| ADH4 | CAAGCCCCTTTGCATTGAAG | 61 |
| CAMK1G | TGGCAGGGAGTGCTACACTG | 62 |
| AKT1 | GACAACCGCCATCCAGACTG | 63 |
| ARID4A | GAAAAGGCTGGTGAAAGTTA | 64 |
| 3EX3 | GAAGACCGCCCTTTGGGAGG | 65 |
| C22orf39 | GAAGCCTTGCACAGAGCCTG | 66 |
| C22orf39 | GAGTCTTGAAGATATCAGGA | 67 |
| CAMK1G | GCGGGGTGTCTACACAGAGA | 68 |
| TOP2A | TAATCAGCAAGCCTTTGATG | 69 |
| COTL1 | ACTCCGCTCCCTGCTCGCCG | 70 |
| DAB2IP | GGAGTTGATGATCTTGCAGA | 71 |
| FES | GCATTTGCTGCAGGACCCCG | 72 |
| FXR2 | ATAATGACAAGAAGAACCCC | 73 |
| GPX6 | CCTAAAGCCTCAAAATAGGA | 74 |
| HIRA | GAAGCCTTGCACAGAGCCTG | 75 |
| HIRA | GAGTCTTGAAGATATCAGGA | 76 |
| INO80C | TTAGCTGGCTTAAAGGATGG | 77 |
| KDM3B | GGAATGCCAGTGGAGAGCCA | 78 |
| LYN | TGAAAGACAAGTCGTCCGGG | 79 |
| SPOP | GTAGCACCAACTCTCAGCTA | 80 |
| MTA2 | GGCCCTAGAGAAGTATGGGA | 81 |
| NPM2 | GGAGGACAAGAAGATGCAGC | 82 |
| NPM2 | GGGAAATGCGCACCATGGGG | 83 |
| PDK3 | TAAGAGCCCTGAGGATCCAC | 84 |
| PFKFB4 | GAGCTACGTGGTGAACCGTG | 85 |
| RPS6KA3 | GGATGAACCTATGGGAGAGG | 86 |
| SIN3A | GCAGATGCCAGCAAACATGG | 87 |
| SMYD1 | AGGAGGAGCAGAAGGACCTG | 88 |
| TPK1 | GGCACTTAGTAAAGTCAGTG | 89 |
| TPK1 | AAGGCTGTCCAACAGGAATA | 90 |
| CUL3 | GAGCATCTCAAACACAACGA | 94 |
| CUL3 | CGAGATCAAGTTGTACGTTA | 95 |
| CUL3 | TCATCTACGGCAAACTCTAT | 96 |

### Example 3

### Bioinformatic Analysis of Target Gene Hits that Upregulate HBF

Multiple bioinformatic analyses were used to identify specific pathways, complexes and tissue specific expression patterns that were enriched in the top targets that significantly upregulate HbF protein levels.

### Protein Complex Analysis:

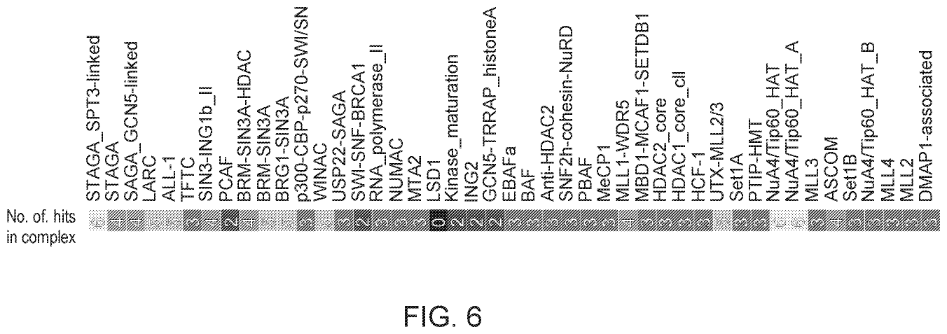

To identify protein complexes with multiple targets that upregulate HbF, top targets identified by the methods described above were overlapped with existing protein complex annotations (CORUM protein complex annotations (Giurgiu M et al, Nucleic Acids Research)). This analysis identified several complexes with multiple targets. These complexes and the number of targets identified as components of each complex are provided in FIG. 6. The overlap of

complex annotations and targets identified using methods 2 and 3 are displayed in Table 3 and Table 4.

TABLE 3
Protein complexes with multiple subunits identified as targets (method 2) that upregulate HbF

| Complex Name | hits_in_cornplex |
|---|---|
| STAGA_compiexSP-13-iinked | TADA3; TAF6L; TADA1; TAF5L; ATXN7L3; TAF 10; |
| | SAP130 |
| STAGA_complex | TADA3; TAF6L; TADA1; TAF5L; TAF10 |
| SAGA_complex,_GCN5-linked | TADA3; TAF6L; TAF5L; ATXN7L3; TAF10 |
| LARC_compIex_(LCR- | MBD3; SMARCB1; SMARCC1; ARID1A; MTA2 |
| associated remodeling complex) | |
| ALL-1_supercomplex | SIN3A; MBD3; SMARCB1; SMARCC1; MTA2 |
| TFTC_complex_(TATA- | TADA3; TAF6L; TAF5L; TAF10 |
| binding_protein-free_TAF-H- | |
| containing complex) | |
| SIN3-ING1b_complex_11 | SIN3A; SMARCB1; SMARCC1; ARID1A |
| PCAF_complex | TADA3; TAF6L; TAF5L; TAF10 |
| Nop56p-associated_pre- | MYBBP1A; FBL; NAP1L1 ; RPL27 |
| rRNA_complex | |
| BRM-SIN3A_complex | SIN3A; SrtflARCB1; SMARCC1; ARID1A |
| BRM-SIN3A-HDAC_complex | SIN3A; SMARCB1; SMARCC1; ARID1A |
| BRG1-SIN3A_complex | SIN3A; SMARCB1; SMARCC1; ARID1A |
| p300-CBP-p270- | SMARCB1; SMARCC1; ARID1A |
| SWI/SNF_complex | |
| WINAC_complex | SMARCB1; SMARCC1; ARID1A |
| USP22-SAGA_complex | TADA3; ATXN7L3; TAF10 |
| Spliceosome | SPEN; SRSF3; PRPF4B |
| SWI- | SMARCB1; SMARCC1; ARID1A |
| SNF_chromatin_remodeling-related- | |
| BRCA1_complex | |
| RNA_polymerase_II_complex,_ | CDK8; SMARCB1; SMARCC1 |
| incomplete_(CDK8_compIex),_chromatin_ | |
| structure_modifying | |
| RNA_polymerase_II_complex,_ | CDK8; SMARCB1; SMARCC1 |
| chromatin_structure_modifying | |
| NUMAC_complex_(nucleosomal_ | SMARCB1; SMARCC1; ARID1A |
| methylation_activator_complex) | |
| MTA2_complex | SIN3A; MBD3; MTA2 |
| LSDl_complex | HMG20B; HMG20A; GIBP1 |
| Kinase_maturation_complex_1 | MAP2K5; YWHAE; YWHAZ |
| ING2_complex | SIN3A; ARID4A; SAP130 |
| GCN5- | TADA3; TAF5L; TAF-10 |
| TRRAP_histone_acetyltransferase_ | |
| complex | |
| EBAFa_complex | SMARCB1; SMARCC1; ARID1A |
| CEN_complex | FBL; SSRP1; CUL4A |
| BAF_cornplex | SMARCB1; SMARCC1; ARID1A |
| Anti-HDAC2_complex | HMG20B; SIN3A; MTA2 |
| ZNF304-corepressor_complex | DNMT1; SETDB1 |
| Ubiguitin_E3 _ligase_(SPOP,_D | SPOP; CUL3 |
| AXX,_CUL3) | |
| Ubiguitin_E3_ligase_(H2AFY,_ | SPOP; CUL3 |
| SPOP,_CUL3) | |
| Ubiquitin_E3_ligase_(DDB1,_D | CUL4B; CUL4A |
| DB2,_CUL4A,_CUL4B,_RBX1) | |
| Ubiquitin_E3_ligase_(BMI1,_S | SPOP; CUL3 |
| POP,_CUL3) | |
| Toposome | SSRP1; TOP2A |
| SNF2h-conesin- | MBD3; MTA2 |
| NuRD_complex | |
| SIN3-SAP25_complex | SIN3A; SAP130 |
| SHARP-CtBP_complex | CTBP1; SPEN |
| SHARP-CtBP1-CtIP_complex | CTBP1; SPEN |
| SHARP-CtBP1-CtIP-RBP- | CTBP1; SPEN |
| Jkappa_corepressor_complex | |
| SETDB1- | ATF7IP; SETDB1 |
| containing_HMTase_complex | |
| Polycomb_repressive_complex | PHC2; CBX4 |
| (PRC1,_hPRC-H1) | |
| PBAF_complex_(Polybromo_ | SMARCB1; SMARCC1 |
| and_BAF_containing_complex) | |
| NCOR1_compiex | SMARCB1; SMARCC1 |
| NCOA6-DNA-PK-Ku- | PARP1; PRKDC |
| PARP1_complex | |
| Mi2/NuRD_complex | MBD3; MTA2 |
| Mi-2/NuRD-MTA2_complex | MBD3; MTA2 |
| MeCP1_complex | MBD3; MTA2 |
| MLL1-WDR5_complex | INO80C; MGAM |
| MBD1-MCAF1- | ATF7IP; SETDB1 |
| SETDB1_complex | |
| ITGAV-ITGB3-EGFR_complex | EGFR; ITGB3 |
| ITGA2b-ITGB3-CD47- | ITGB3; SRC |
| SRC_complex | |
| Histone_H3.3_complex | NASP; HIRA |
| HDAC2- | MBD3; MTA2 |
| asscociated_core_complex | |
| HDAC1- | MBD3; MTA2 |
| associated protein complex | |
| HDAC1- | MBD3; MTA2 |
| associated_core_cornplex_cII | |
| HCF-1_complex | SIN3A; SETD1A |
| FIB- | FBL; PRMT1 |
| associated_protein_complex | |
| Exosome | EXOSC1; EXOSC9 |
| Emerin_complex_52 | HDGF; YWHAE |
| Emerin_complex_32 | SMARCB1; SMARCC1 |
| Emerin_complex_25 | YWHAE; SAP130 |
| Emerin_complex_24 | RB1; SAP130 |
| EGFR- | EGFR; PIK3C2A |
| containing_signaling_complex | |
| EBAFb_complex | SMARCB1; SMARCC1 |
| CtBP_cornplex | CTBP1; CBX4 |
| CDC5L_complex | PRKDC; TOP2A |
| ATAC_compiex,_YEATS2- | TADA3; YEATS2 |
| linked | |
| ATAC_complex,_GCN5-linked | TADA3; YEATS2 |
| ARC_complex | CDK8; ACAD8 |
| pRb2; p130- | DNMT1 |
| muftirnoecular_complex_(DNMT1,_E2F | |
| 4,_SuV391-11 ,_HDAC1,_RBL2) | |
| p32-CBF-DNA_complex | NFYC |
| p300-CBP-p270_complex | ARID1A |
| p27-cyclinE-Cdk2_-_ | CDKN1B |
| Ubiquitin_E3_ligase_(SKP1A,_SKP2,_ | |
| CUL1,_CKS1B,_RBX1)_complex | |
| p27-cyclinE-CDK2_complex | CDKN1B |
| p21(ras)GAP-Fyn-Lyn- | LYN |
| Yes complex, thrombin stimulated | |
| p130Cas-ER-alpha-cSrc- | SRC |
| kinase-_PI3-kinase_p85- | |
| subunit_complex | |
| hNURF_complex | SMARCA1 |
| eN0S-HSP90- | AKT1 |
| AKT complex,_VEGF_induced | |
| c-Abl-cortactin- | MYLK |
| nrnMLCK_complex | |
| anti-BHC110_wmplex | HMG20B |
| WRN-Ku70-Ku80- | PARP1 |
| PARP1 | complex |
| WDR2O-USP46-UAF1_complex | USP46 |
| Vigilin-DNA-PK- | PRKDC |
| Ku_antigen_complex | |
| VEcad-VEGFR_complex | FLT4 |
| Ubiquitin_E3_ligase_(DET1,_D | CUL4A |
| DB1,_,CUL4A,_RBX1,_COP1) | |
| Ubiquitin_E3_ligase_(DDIT4,_D | CUL4A |
| DB1,_BTRC,_CUL4A) | |
| Ubiquitin_E3 Jigase_(DDB1,_C | CUL4A |
| UL4A,_RBX1) | |
| Ubiquitin_E3_ligase_(CUL3,_K | CUL3 |
| LHL3,_WNK4) | |
| Ubiquitin_E3_ligase_(CUL3,_K | CUL3 |
| LHL3,_WNK1) | |
| Ubiquitin_E3_Jigase_(CUL3,_K | CUL3 |
| Li-1L3) | |
| Ubiquitin_E3_ligase_(CSN1,_C | CUL3 |
| SN8,_HRT1,_SKP1,_SKP2,_CUL1,_C | |
| UL2,_CUL3) | |
| Ubiquitin_E3 _ligase_(CHEK1,_ | CUL4A |
| CUL4A) | |
| Ubiquitin_E3 _ligase_(CDT1,_D | CUL4A |
| DB1,_,CUL4A,_RBX1) | |
| Ubiquitin_E3_ligase_(AHR,_AR | CUL4B |
| NT,_DDB1,_TBL3,_CUL4B,_RBX1) | |
| UTX-MLL2/3_complex | KMT2C |
| USP46-UAF1_cornplex | USP46 |
| ULK2-ATG13- | ULK2 |
| RB1CC1_complex | |
| Tacc1-chTOG- | AURKA |
| AuroraA_complex | |
| TRIM27-RB1_complex | RB1 |
| TRIB3-DDIT3_complex | TRIB3 |
| TRBP_containing_complex_(DI | RPL27 |
| CER,_RPL7A,_EIF6,_MOV10_and_sub | |
| units_of_the_60S_ribosomal_particle) | |
| TNF-alpha/NF- | FBL |
| kappa_B_signalino_complex_6 | |
| TNF-alpha/NF- | TBK1 |
| kappa_B_signaling_complex_10 | |
| TIP5-DNMT-HDAC1_complex | DNMT1 |
| TFIID_complex,_B-cell_specific | TAF10 |
| TFIID_complex | TAF10 |
| TFIID-beta_cornpIex | TAF10 |
| TCL1(trimer)-AKT1_complex | AK-r1 |
| Succinyl- | SUCLG1 |
| CoA_synthetase,_GDP-forming | |
| Succinyl- | SUCLG1 |
| CoA synthetase, ADP-forming | |
| Set1A_complex | SETD1A |
| SWIISNF_chromatin- | SIN3A |
| remodeling complex | |
| SNX_complex (SNX1a,_SNX2,_ | EGFR |
| SNX4,_EGFR) | |
| SNF2L-RSF1_complex | SMARCA1 |
| SMCC_complex | CDK8 |
| SMAR1-HDAC1-S1N3A- | SIN3A |
| SIN3B_repressor_complex | |
| SMAR1-HDAC1-SIN3A-SIN3B- | SIN3A |
| p107-p130_repressor_cornolex | |
| SMAD3-cSKI-SIN3A- | SIN3A |
| HDAC1_complex | |
| SKl-NCOR1-SIN3A- | SIN3A |
| HDAC1_complex | |
| SIN3_complex | SIN3A |
| SIN3-ING1b_complex_I | SIN3A |
| SHARP-CtIP-RBP- | SPEN |
| Jkappa_complex | |
| SH3KBP1-CBLB- | EGFR |
| EGFR_complex | |
| SETDB1-DNMT3B...complex | SETDB1 |
| SETDB1-DNMT3A_complex | SETDB1 |
| SERCA2a-alphaKAP-CafV1- | CALM1 |
| CaMKII_complex | |
| Ribosome; _cytoplasmic | RPL27 |
| Replication-coupled CAF-1- | SETDB1 |
| MBD1-ETDB1_complex | |
| Rb-tal-1-E2A-Lmo2- | RB1 |
| Ldb1_complex | |
| Rb-HDACl_complex | RB1 |
| RasGAP-AURKA- | AURKA |
| survivin_complex | |
| Rap1_complex | PARP1 |
| RSmad_complex | SMARCC1 |
| RIN1-STAM2- | EGFR |
| EGFR_oornplex,_EGF_stimulated | |
| REST-CoREST- | SIN3A |
| mSIN3A_complex | |
| RC_complex_during_S- | PARP1 |
| phase_of_cell_cycle | |
| RC_cornplex_during_G2/M- | PARP1 |
| phase_of_cell_cycle | |
| RBP-Jkappa-SHARP_compiex | SPEN |
| RB1-TFAP2A_complex | RB1 |
| RB1-HDAC1-BRG1_complex | RB1 |
| RB1(hypophosphorylated)- | RB1 |
| E2F4_compIex | |
| RB-E2F1_complex | RB1 |
| RAF1-MAP2K1- | YWHAE |
| YWHAE | complex |
| Polycystin- | SRC |
| 1_multiprotein_complex_(ACTN1, CDH | |
| 1, SRC, JUP, VCL, | CTNNB1,_FTXN,_ |
| BEAR1,_PKD1,_PTK-2,_TLN1) | |
| Polycomb_repressive_complex_ | EZH2 |
| 4_(PRC4) | |
| Polyoornb_repressive_complex_ | EZH2 |
| 2_(PRC2) | |
| Polycomb_repressive_complex | CBX4 |
| Phosphorylase_kinase_complex | CALM1 |
| PU.1-Sl N3A-HDAC_complex | SIN3A |
| PTIP-HMT_complex | KMT2C |
| PTEN-NHERF1- | EGFR |
| EGFR_complex | |
| PRMT2_tiorno- | PRMT2 |
| oligomer complex | |
| PRMTl_complex | PRMT1 |
| PLC-gamma-2-SLP-76-Lyn- | LYN |
| Grb2_complex | |
| PLC-gamma-2-Lyn-FcR- | LYN |
| gamma | complex |
| PKA_(RII-alpha_and_RII-beta)- | PRKAR2B |
| AKAP5-ADRB1_complex | |
| PCNA_complex | CDKN1A |
| PCNA-p21_complex | CDKN1A |
| P53-BARD1-Ku70_complex | BARD1 |
| NuRD.1_complex | MBD3 |
| NuA4/Tip60_HAT_complex | MEAF6 |
| NuA4/Tip60-HAT_complex_A | MEAF6 |
| NRP2-VEGFR3_cornplex | FLT4 |
| NK-3-Groucho-HIPK2-SIN3A- | SIN3A |
| RbpA48-HDAC1_complex | |
| NCOR2_complex | SIN3A |
| NCOR-SIN3-RPD3_complex | SIN3A |
| NCOR-SIN3-HDAC1_complex | SIN3A |
| NCOR-SIN3-HDAC- | SIN3A |
| HESX1_complex | |
| NAT_complex | CDK8 |
| Mi2/NuRD-BCL6- | MBD3 |
| MTA3_complex | |
| Mediator complex | CDK8 |
| MeCP2-SIN3A-HDAC_complex | SIN3A |
| MIAl_mmplex | MBD3 |
| MSL_complex | MSL3 |
| MRG15-PAM14-RB_complex | RB1 |
| MLL3_complex | KMT2C |
| MGC1-DNA-PKcs-Ku_complex | PRKDC |
| MBD1-MCAFcomplex | ATF7IP |
| MAP2K1-BRAF-RAF1-YWHAE- | YWHAE |
| KSR1_complex | |
| MAD1-mSin3A- | SIN3A |
| HDAC2_complex | |
| Kinase_maturation_complex_2 | TBK1 |
| ITGB3-ITGAV-VTN_complex | ITGB3 |
| I1GB3-ITGAV-CD47_complex | ITGB3 |
| ITGAV-ITGB3_complex | ITGB3 |
| ITGAV-ITGB3-THBS1_complex | ITGB3 |
| ITGAV-ITGB3-SPP1_complex | ITGB3 |
| ITGAV-ITGB3- | ITGB3 |
| SLC3A2_complex | |
| ITGAV-ITGB3-PXN- | ITGB3 |
| PTK2b_complex | |
| ITGAV-ITGB3- | ITGB3 |
| PPAP2b complex | |
| ITGAV-ITGB3-NOV_complex | ITGB3 |
| ITGAV-ITGB3-LAMA4_complex | ITGB3 |
| ITGAV-ITGB3- | ITGB3 |
| COL4A3_complex | |
| ITGAV-ITGB3-0D47- | ITGB3 |
| FCER2_complex | |
| ITGAV-ITGB3- | ITGB3 |
| ADAM23_complex | |
| ITGAV-ITGB3- | ITGB3 |
| ADAM15_complex | |
| ITGA5-ITGB3- | ITGB3 |
| COL6A3_complex | |
| ITGA2b-ITGB3-TLN1_complex | ITGB3 |
| ITGA2b-ITGB3-CD9_complex | ITGB3 |
| ITGA2b-ITGB3-CD9-GP1b- | ITGB3 |
| CD47_complex | |
| ITGA2b-ITGB3-CD47- | ITGB3 |
| FAK_complex | |
| ITGA2B-ITGB3_complex | ITGB3 |
| ITGA2B-ITGB3- | ITGB3 |
| ICAM4_complex | |
| ITGA2B-ITGB3-FN1- | ITGB3 |
| TGM2_complex | |
| ITGA2B-ITGB3-F11R_complex | ITGB3 |
| ITGA2B-ITGB3-CIB1_complex | ITGB3 |
| ITAGV-ITGB3-F11R_complex | ITGB3 |
| INO80_chromatin_remodeling_ | INO80C |
| complex | |
| ING5_complex | MEAF6 |
| ING4_complex_(ING4,_MYST2,_ | MEAF6 |
| C1or-1149,_PHF17) | |
| ING4_complex_(ING4 ,_MYST2,_ | MEAF6 |
| C1or1149,_PHF16) | |
| ING4_complex_(ING4,_MYST2,_ | MEAF6 |
| C1orf149,_PHF15) | |
| IGF1R-CXCR4-GNA12- | IGF1R |
| GNB1_complex | |
| Histone_H3.1_complex | NASP |
| HUIC_complex | BARD1 |
| HSP90-CIP1-FKBPL_complex | CDKN1A |
| HMGB14-IMGB2-HSC70- | GAPDH |
| ERP60-GAPDH_complex | |
| HES1_promoter- | CDK8 |
| Notch_enhancer_complex | |
| HERP1/HEY2-NCOR- | SIN3A |
| SIN3A_complex | |
| HBO1_complex | MEAF6 |
| H2AX_complex_I | PARP1 |
| H2AX_complex; _isolateg_from_ | SSRP1 |
| cells_without_IR_exposure | |
| G_alpha-13-Flax-1-cortactin- | AKT1 |
| Rac_complex | |
| GAIT_complex | GAPDH |
| FGFR2-c-Cbl-Lyn-Fyn_complex | LYN |
| FGFR1c-KL_complex | FGFR1 |
| FGF23-FGFR1c-KL_cornolex | FGFR1 |
| FGF21-FGFR1c-KLB_complex | FGFR1 |
| FE65-ISHZ3-HDACl_complex | ISHZ3 |
| FA_complex_(Fanconi_anemia_ | RMI1 |
| complex) | |
| FACT_complex,_UV-activated | SSRP1 |
| FACT_complex | SSRP1 |
| FACT-NEK9_complex | SSRP1 |
| F1F0- | ATP5F1C |
| ATP_synthase,_mitochondrial | |
| Elongator_holo_complex | ELP2 |
| EcV,_complex_ JECSIT,_MT- | GAPDH |
| CO2,_GAPDH,_TRAF6,_NDUFAF1) | |
| ETS2-SMARCA4-INI1_complex | SMARCB1 |
| ERBB3-SPG1_complex | ERBB3 |
| EGFR-CBL-GRB2_complex | EGFR |
| EED-EZH_polycomb_complex | EZH2 |
| EED-EZH2_complex | EZH2 |
| EED-EZH- | EZH2 |
| YY1_polycomb_complex | |
| DRD4-FLHL12-CUL3_complex | CUL3 |
| DNMT3B_complex | SIN3A |
| DNMT1-G9a_complex | DNMTI |
| DNMT1-G9a-PCNA_complex | DNMTI |
| DNA_synthesome_complex_(1 | TOP2A |
| 7_subunits) | |
| DNA-PK-Ku_complex | PRKDC |
| DNA-PK-Ku-elF2-NF90- | PRKDC |
| NF45_complex | |
| DHX9-ADAR-vigilin-DNA-PK- | PRKDC |
| Ku_antigen complex | |
| DDN-MAGI2- | MAGI2 |
| SH3KBP1_complex | |
| DDB2_complex | CUL4A |
| DA_complex | TAF10 |
| DAB_complex | TAF10 |
| CyclinD3-CDK4-CDK6- | CDKN1A |
| p21_complex | |
| Condensin_I-PARP-1- | PARPI |
| XRCCI complex | |
| CoREST-F-IDAC_complex | FiMG2013 |
| Cell_cycle_kinase_complex_C | CDKN1A |
| DK5 | |
| Cell_cycle_kinase_complex_C | CDKN1A |
| DK4 | |
| Cell_cycle_kinase_complex_C | CDKN1A |
| DK2 | |
| Cell_cycle_kinase_cornplex_C | CDKN1A |
| DC2 | |
| C_complex_spliceosome | PRPF4B |
| CUL4B-DDB1- | CUL4B |
| WDR26_complex | |
| CUL4B-DDB1-TLE3_complex | CUL4B |
| CUL4B-DDB1-TLE2_complex | CUL4B |
| CUL4B-DDB1-TLEl_complex | CUL4A |
| CUL4B-DDB1- | CUL4B |
| GRWD1_complex | |
| CUL4B-DDB1-DTL- | CUL4B |
| CSN_complex | |
| CUL4A-DDB1- | CUL4A |
| WDR61_complex | |
| CUL4A-DDB1-WDR5_compiex | CUL4A |
| CUL4A-DDB1- | CUL4A |
| WDR5B_complex | |
| CUL4A-DDB1- | CUL4A |
| WDR57_complex | |
| CUL4A-DDB1-RBBP5_complex | CUL4A |
| CUL4A-DDB1-EED_complex | CUL4A |
| CUL4A-DDB1-DTL_complex | CUL4A |
| CSA complex | CUL4A |
| CSA-POLIIa_complex | CUL4A |
| CS-MAP3K7IP1- | CS |
| MAP3K7IP2_complex | |
| CNK1-SRC-RAF1_complex | SRC |
| CHTOP-methylosome_complex | PRMT1 |
| CERF_complex_(CECR2- | SMARCA1 |
| containing_remodeling_factor_complex) | |
| CEP164-TTBK2_complex | TIBK2 |
| CEBPE-E2F1-RB1_complex | RBI |
| CDK8-CyclinC- | CDK8 |
| Mediator_complex | |
| CD2O-LCK-LYN-FYN- | LYN |
| p75/80_complex,_(Raji_human_B_cell_ | |
| line) | |
| CCDC22-COMMD8- | CUL3 |
| CUL3_complex | |
| CBF-DNA_complex | NFYC |
| CAS-SRC-FAK_complex | SRC |
| CAND1-CUL4B-RBXl_complex | CUL4B |
| CAND1-CUL4A-RBX1_complex | CUL4A |
| CAND1-CUL3-RBX1_complex | CUL3 |
| CALM1-_ | CALM1 |
| KCNC)4(splice_variant_2)_complex | |
| CALM1- | CALM1 |
| KCNQ4(splice_variant_1)_complex | |
| BRMS1-SIN3-HDAC_cornplex | SIN3A |
| BRCAl_C_complex | BARD1 |
| BRCAl_B_complex | BARD1 |
| BRCA1_k_complex | BARD1 |
| BRCA1-CtIP-CtBP_complex | CTBP1 |
| BRCA1-BARD1- | BARD1 |
| UbcH7c_complex | |
| BRCA1-BARD1- | BARD1 |
| UbcH5c complex | |
| BRCA1-BARD1- | BARD1 |
| POLR2A_complex | |
| BRCA1-BARD1-BRCA2- | BARD1 |
| DNA_damage_complex_III | |
| BRCA1-BARD1-BACH1 - | BARD1 |
| DNA_damage_complex_II | |
| BRCA1-BARD1-BACH1- | BARD1 |
| DNA_damage_complex_I | |
| BRAFT_complex | RMI1 |
| BRAF53-BRCA2_complex | HMG20B |
| BRAF-RAF1-14-3-3_complex | YWHAZ |
| BRAF-MAP2K1-MAP2K2- | YWHAE |
| YWHAE_complex | |
| BMI1-HPH1-HPH2_complex | PHC2 |
| BLM_cornplex_III | RMI1 |
| BLM_complex_II | RMI1 |
| BHC_complex | HMG2013 |
| BARD1-BRCA1- | BARDI |
| CSTF_complex | |
| BARDI-BRCAI- | BARDI |
| CSTF64_complex | |
| B-WICH_complex | MYBBP1A |
| Artemis-DNA-PK_complex | PRKDC |
| Akt-PHLPP1-PHLPP2-FANCI- | AKTI |
| FANCD2-USP1-UAFl_complex | |
| AURKA-INPP5E_complex | AURKA |
| AURKA-HDAC6_cilia- | AURKA |
| disassembly complex | |
| ASFI- | HIRA |
| interacting_protein_complex | |
| ASFI- | NASP |
| histone_containing_complex | |
| ASCOM_complex | KMT2C |
| ARC92-Allediator_complex | CDK8 |
| ARC-L_complex | CDK8 |
| AR-AKT-APPL_complex | AKTI |
| AMY-I-S-AKAP84-RII- | PRKAR2B |
| beta_complex | |
| 60S_ribosomal_subunit,_cytoplasmic | RPL27 |
| 17S_U2_snRNP | HMG20B |

TABLE 4
Protein complexes with multiple subunits identified as targets (method 3) that upregulate HbF

| Complex Name | hits_in_complex |
|---|---|
| STAGA_complex,_SPT3-linked | TAF6L; TADA1; KAT2A; ATXN7L3; SAP130; TRRAP |
| NuM/Tip6O_HAT_complex | KAT5; BRD8; MEAF6; EPC1; YEATS4; TRRAP |
| NuMiTip6O-HAT_complex_A | KAT5; BRD8; MEAF6; EPC1; YEATS4; TRRAP |
| WINAC_complex | 5UPT16H; SMARCB1; SMARCD1; ARID1A; BAZ1B |
| UTX-MLL2/3_complex | N4BP2; KMT2C; RBBP5; KMT2D; ASH2L |
| Spliceosome | CDK12; PPM1G; SRSF1; SRSF3; PRPF4B |
| Nop56p-associated_pre-rRNA_complex | MYBBP1A; FBL; NAP11.1; RPL27; H1FX |
| LARC_complex_(LCR- | MBD3; GATAD2B; SMARCB1; ARID1A; MTA2 |
| associated_remodeling_.amplex) | |
| BRM-SIN3A_complex | SIN3A; SMARCB1; SMARCD1; SMARCD3; ARID1A |
| BRG1-SIN3A_complex | SIN3A; SMARCB1; SMARCD1; SMARCD3; ARID1A |
| ALL-1_supercomplex | SIN3A; MBD3; RBBP5; SMARCB1; MTA2 |
| STAGA_complex | TAF6L; TADA1; KAT2A; TRRAP |
| SIN3-ING1b_complex_II | SIN3A; SMARCB1; SMARCD1; ARID1A |
| SAGA_complex,_GCNS-linked | TAF6L; KAT2A; ATXN713; TRRAP |
| MLL1-WDR5_complex | INO80C; E2F6; RBBP5; ASH2L |
| BRM-SIN3A-HDAC_complex | SIN3A; SMARCB1; SMARCD1; ARID1A |
| ASCOM_complex | KMT2C; RBBP5; KMT2D; ASH2L |
| p300-CBP-p270-SWI/SNF_complex | CREBBP; SMARCB1; ARID1A |
| USP22-SAGA_complex | KAT2A; ATXN7L3; TRRAP |
| TFTC_cornplexiTATA-binding_protein- | TAF6L; KAT2A; TRRAP |
| free TAF-II-containing_complex) | |
| Set1B_complex | CXXC1; RBBP5; ASH2L |
| Set1A_complex | CXXC1; RBBP5; ASH2L |
| SNF2h-cohesin-NuRD_complex | BAZ1A; MBD3; MTA2 |
| RNA_polymeraseil_complex,_chromatin_ | CREBBP; SMARCB1; SMARCD1 |
| structure_modifying | |
| PTIP-HMT_complex _ | KMT2C; RBBP5; ASH2L |
| PBALcomplex_(Polybromo-_ | |
| and BAF_containing_complex) | PBRM1; SMARCD1; SMARCD1 |
| NuA4/Tip6O-HAT_complex_B | KAT5; EPCLTRRAP |
| NUMAC_complexinucleosornal_methylation_ | |
| activator_complex) | SMARCB1; SMARCD1; ARID1A |
| MeCP1_complex | MBD3; GATAD2B; MTA2 |
| MTA2_complex | SIN3A; MBD3; MTA2 |
| MLL4_complex | RBBP5; KMT2D; ASH2L |
| MLL3_complex | KMT2C; RBBP5; ASH2L |
| MLL2_complex | RBBP5; KMT2D; ASH2L |
| NIBD1-MCAF1-SETDB1_complex | MBD1; ATF7IP; SETDB1 |
| HDAC2-asscociated_core_complex | MBD3; GATAD2A; MTA2 |
| HDAC1-associated_core_complex_cII | MBD3; GATAD2A; MTA2 |
| HCF-1_complex | 5IN3B; SIN3A; ASH2L |
| EBAFa_complex | SMARCB1; SMARCD1; ARID1A |
| DMAP1-associated_complex | BRD8; EPC1; TRRAP |
| CEN_complex | SUPT16H; FBL; SSRP1 |
| CDC5L_complex | PRKDC; SFPQ; SRSF1 |
| BAF_complex | SNIARCB1; SMARCD1; ARID1A |
| Anti-HDAC2_complex | SIN3A; ZMYM3; MTA2 |
| p300-CBP-p270_complex | CREBBP; ARID1A |
| WRA_complex_(WDR5,_RBBP5,_ASH2L) | RBBP5; ASH2L |
| WRAD_complex_(WDR5,_RBBP5,_ASH | RBBP5; ASH2L |
| 2L,_DPY30) | |
| Ubiquitin_E3 _ligase_(CSN1,_CSN8,_HRT1,_ | SKP1; CUL3 |
| SKP1,_SKP2,_CUL1,_CUL2,_CUL3) | |
| TIP60_histone_acetylase_complex | KAT5; TRRAP |
| TFTC- | KAT2A; TRRAP |
| type_histone_acetyl_transferase_complex | |
| SWI-SNF_chromatin_remodeling- | SMARCB1; ARID1A |
| related-BRCA1_complex | |
| SRC-3_complex | CREBBP; NCOA3 |
| SMAR1-HDAC1-SIN3A- | SIN3B; SIN3A |
| SIN3B_repressor_complex | |
| SMAR1-HDAC1-SIN3A-SIN3B-p107- | SIN3B; SIN3A |
| p130_repressor_complex | |
| SKI-NCOR1-SIN3A-HDAC1_complex | SIN3A; NCOR1 |
| SIN3-SAP25_complex | SIN3A; SAP130 |
| SETDB1-containing_HMTase_complex | ATHIP; SEIDB1 |
| SERCA2a-alphaKAP-CaM- | CALM1; CAMK2A |
| CaMKII_complex | |
| Ribosome,_cytoplasmic | RPL27; RPS4X |
| Replication-coupled_CAF-1-MBD1- | MBD1; SETDB1 |
| ETDB1_complex | |
| RSmad_complex | CREBBP; NCOA3 |
| RC_complex_during_S- | PARP1; POLD1 |
| phase_of_cell_cycle | |
| RC_complex_during_G2/m- | PARP1; POLD1 |
| phase_of_cell_cycle | |
| Polycomb_repressive_complex_4_(DRC4) | EZH2; EED |
| Polycomb_repressive_complex_2_(PRC2) | EZH2; EED |
| PCAF_complex | TAF6L; TRRAP |
| NIF1-ASH2L-RBBPS-WDR5_complex | RBBB5; ASH2L |
| NCOR2_complex | SIN3A; NCOR1 |
| NCOR1_complex | SMARCB1; NCOR1 |
| NCOR-SIN3-RPD3_cornplex | SIN3B; SIN3A |
| NCOR-SIN3-HDAC-HESX1_complex | SIN3B; SIN3A |
| NCOA6-DNA-PK-Ku-PARPl_complex | PARP1; PRKDC |
| Multisubunit_ACTR_coactivator_complex | CREBBP; NCOA3 |
| Mi2/NuRD_complex | MBD3; MTA2 |
| Mi-2/NuRD-MTA2_complex | NIBD3; MTA2 |
| Menin- | RBB135; ASH2L |
| associated_histone_methyltransferase_ | |
| complex | |
| MLL1_core_complex | RBBP5; ASH2L |
| MLL1_complex | RBBP5; ASH2L |
| MLL-HCF_complex | RBBP5; ASH2L |
| MBD1-MCAF_complex | MBD1; ATF7IP |
| Kinase_maturation_complex_1 | YWHAE; YWHAZ |
| INO80_chromatin_remodeling_complex | INO80C; INO80 |
| ING4_complex_(ING4,_MYST2,_C1orf149,_ | ING4; MEAF6 |
| PHF17) | |
| ING4_complex_(ING4,_MYST2,_C1orf149,_ | ING4; MEAF6 |
| PHF16) | |
| ING4_complex_ING4,_MYST2,_C1orf149,_ | ING4; MEAF6 |
| PHF15) | |
| ING2_complex | SIN3A; SAP130 |
| HDAC1-associated_protein_complex | MBD3; MTA2 |
| HBO1_complex | ING4; MEAF6 |
| H2AX_complex,_isolatedirom_cells_ | SUPT16H; SSRP1 |
| without_IR_exposure | |
| GCN5- | KAT2A; TRRAP |
| TRRAP_histone_acetyltransferase_ | |
| complex | |
| FIB-associated_protein_complex | FBL; PRMT1 |
| FACT_complex,_UV-activated | SUPT16H; SSRP1 |
| FACT_complex | SUPT16H; SSRP1 |
| FACT-NEK9_complex | SUPT16H; SSRP1 |
| Exosome | EXOSC1; EXOSC9 |
| Emerin_complex_52 | HDGF,YWHAE |
| Emerin _complex_25 | YWHAE; SAP130 |
| EED-EZH_polycomb_complex | EZH2; EED |
| EED-EZH2_complex | EZH2; EED |
| EED-EZH-YY1_polycomb_complex | EZH2; EED |
| EBAFI3_complex | SMARCB1; SMARCD1 |
| E2F-6_complex | E2F6; PCGF6 |
| CyclinD3-CDK4-CDK6_complex | CDK4; CDK6 |
| CyclinD3-CDK4-CDK6-p21_complex | CDK4; CDK6 |
| C_complex_spliceosome | SRSH; PRPF4B |
| BRMS1-SIN3-HDAC_complex | SIN3B; SIN3A |
| BRCAl-BARD1-BRCA2- | |
| DNA_damage_complex_III | BARD1; BRCA2 |
| BCOR_complex | BCOR; SKP1 |
| B-WICH_complex | MYBBP1A; BAZ1B |
| ATAC_complex,_YEATS2-linked | A0.18549.1; KAT2A |
| ATAC_complex,_GCN5-linked | AC118549.1; KAT2A |
| transcription_factor_IIC_multisubunit_ | GTF3C4 |
| complex | |
| snRNP-free_U1A_(SF-A)_complex | SFPQ |
| p54(nrb)-PSF-matrin3_complex | SFPQ |
| p400-associated_complex | TRRAP |
| p34(SEI-1)-CDK4-CyclinD2_complex | CDK4 |
| p27-cyclinE-Cdk2_- | |
| _Ubiquitin_E3_ligase_(SKP1A,_SKP2,_ | SKP1 |
| CUL1,_CKS1B,_RBX1)_complex | |
| p21(ras)GAP-Fyn-Lyn- | LYN |
| Yes_complex,_thrombin_stimulated | |
| p130Cas-ER-alpha-cSrc-kinase-_PI3- | SRC |
| kinase_p85-subunit_complex | |
| c-MYC-ATPase-helicase_complex | TRRAP |
| anti-B1-1C110_complex | ZMYM3 |
| Z01-(beta)cadherin-(VE)cadherin- | |
| VEGFR2_complex | KDR |
| ZNE304-corepressor_complex | SETDB1 |
| XFINA complex_ | ZMYM3 |
| WRN-Ku70-Ku8O-PARP1_complex | PARP1 |
| WICH_complex | BAZ1B |
| Vigilin-DNA-PK-Ku_antigen_complex | PRKDC |
| VEcad-VEGFR_cornplex | KDR |
| VEGFR2-S1PR5-ERK1/2-PKC- | KDR |
| alpha_complex | |
| VEGFR2-S1PR3-ERK1/2-PKC- | KDR |
| alpha_complex | |
| VEGFR2-S1PR2-ERK1/2-PKC- | KDR |
| alpha_complex | |
| VEGFR2-S1PR1-ERK1/2-PKC- | KDR |
| alpha_complex | |
| VEGFA(165)-KDR-NRP1_complex | KDR |
| Ubiquitin_E3_ligaseiSPOP,_DAXX,_CUL3) | CUL3 |
| Ubiquitin_E3_ligaseiSMAD3,_BIRC,_ | SKP1 |
| CULL_SKP1A,_RBX1) | |
| Ubiquitin_E3_ligaseiSKP1A,_SKP2,_ | SKP1 |
| CUL1,_RBX1) | |
| Ubiquitin_E3_ligaseiSKP1A,_SKP2,_ | SKP1 |
| CUL1,_CKS1B,_RBX1) | |
| Ublquitin_E3 _ligase_(SKP1A,_SKP2,_CUL1) | SKP1 |
| Ublquitin_E3 _ligase_(SKP1A,_FBXW8,_ | |
| CUL7,_RBX1) | SKI31 |
| Ubiquitin_E3_ligase_(SKP1A,_FBXVV2,_ | |
| Cal) | SKP1 |
| Ubiquitin_E3_ligase_(SKP1A,_BTRC,_ | SKP1 |
| CUL1) | |
| Ubiquitin_E3_ligase_(SIAH1,_SIP,_SKP1A,_ | SKP1 |
| TBL1X) | |
| Ubiquitin_E3_ligase_(NIPA,_SKPlA,_CUL1,_ | SKP1 |
| RBX1) | |
| Ubiquitin_E3_ligase_(NFKBIA,_FBXW11,_ | SKP1 |
| BTRC,_CUL1,_SKP1A) | |
| Ubiquitin_E3_ligase_(F12AFY,_SPOP,_ | CUL3 |
| CUL3) | |
| Ubiquitin_E3_ligase_(GLMN,_FBXW8,_ | SKP1 |
| SKP1A,_RBX1) | |
| Ubiquitin_E3_ligase_(FBXW7,_CUL1,_ | SKP1 |
| SKP1A,_RBX1) | |
| Ubiquitin_E3_ligase_(FBXW11,_SKP1A,_ | SKP1 |
| CUL1,_RBX1) | |
| Ubiquitin_E3_ligase_(FBXO31,_SKP1A,_ | SKP1 |
| CUL1,_RBX1) | |
| Ubiquitin_E3_ligase_(FBXO18,_SKP1A,_ | SKP1 |
| CUL1,_RBX1) | |
| Ubiquitin_E3_ligase_(CUL3,_KLHL3,_ | CUL3 |
| WNK4) | |
| Ubiquitin_E3_ligase_(CUL3,_KLHL3,_ | CUL3 |
| WNK1) | |
| Ubiquitin_E3_ligase_(CUL3,_KLHL3) | CUL3 |
| Ubiquitin_E3_ligase_(CUL1,_RBX1,_SKP1) | SKP1 |
| Ubiquitin_E3_ligase_(CRY2,_SKP1A,_CUL1,_ | SKP1 |
| FBXL3) | |
| Ubiquitin_E3 _ligase_(CRY1,_SKP1A,_CUL1,_ | SKP1 |
| FBXL3) | |
| Ubiquitin_E3 _ligase_(CDC34,_NEDD8,_ | SKP1 |
| BTRC,_CULL_SKP1A,_RBX1) | |
| Ubiquitin_E3 _ligase_(BM11,_SPOP,_CUL3) | CUL3 |
| URI_complex_(Unconventional_prefoldin_ | SKP1 |
| RPBS_Interactor) | |
| ULK2-ATG13-RB1CC1_complex | ULK2 |
| Toposome | SSRP1 |
| Ternary_complex_(LRRC7,_CAMK2a,_ACTN4) | CAMK2A |
| TRRAP-BAF53-HAT_complex | TRRAP |
| TRIB3-DD1T3_complex | TRIB3 |
| TRBP_containing_complexiDICER,_RPL7A,_ | RPL27 |
| EIFG,_MOV10_and_subunits_of_the_ | |
| 60S_ribosomal_particle) | |
| TNF-alpha/NF- | FBL |
| kappa_B_signaling_complex_6 | |
| TNF-alpha/NF- | SKP1 |
| kappa_B_signaling_complex_S | |
| TNF-alpha/NF- | TBK1 |
| kappa_B_signaling_complex_10 | |
| TNF-alpha/NF- | |
| kappa_B_signaling_complex JCHUK,_B | SKP1 |
| TRC,_NEKB2,_PPP6C,_REL,_CUL1,_IKBK | |
| E,_SAPS2,_SAPS1,_ANKRD28,_RELA,_SKP1) | |
| TFIIIC_containing-TOP1-SUB1_complex | GTF3C4 |
| TCF4-CTNNB1-CREBBP_complex | CREBBP |
| TBPIP/HOP2-MND1_complex | PSMC3IP |
| Succinyl-CoA_synthetase,_GDP-forming | SUCLG2 |
| Stati-alpha-dimer-CBP_DNA- | CREBBP |
| protein_complex | |
| Set/TAF-I_beta-1AF-1_alpha- | ANP32A |
| PP32_complex | |
| SWI/SN F_chromatin- | SIN3A |
| remodeling_complex | |
| STAGA_core_complex | KAT2A |
| SRCAP- | YEATS4 |
| associated_chromatin_remodeling_ | |
| complex | |
| SRC-1_complex | CREBBP |
| SNF2H-BAZ1A_complex | BAZ1A |
| SMAD4-SKI-NCOR..complex | NCOR1 |
| SMAD3-cSKI-SIN3A-HDACl_complex | SIN3A |
| SMAD3-SMAD4-FOXO1_complex | FOXO1 |
| SMAD3-SKI-NCOR_complex | NCOR1 |
| SMAD2-SKI-NCOR_complex | NCOR1 |
| SMAD1-CBP_complex | CREBBP |
| SIN3_complex | SIN3A |
| SIN3-ING1b_complex | SIN3A |
| SETDB1-DNIV1T3B_complex | SETDB1 |
| SETDB1-DNMT3A_complex | SETDB1 |
| Rap1_complex | PARPI |
| RNA_polymerase_II_complex,_incomplete_ | SMARCB1 |
| KDK8_cornplexLchromatin_structure_ | |
| modifying | |
| REST-CoREST-mSIN3A_complex | SIN3A |
| RAF1-MAP2K1-YWHAE_cornplex | YWHAE |
| RAD6A-KCMF1-UBR4_complex | UBE2A |
| Prune/Nm23-H1_complex | NME1 |
| Protein_phosphatase_4_complex | PPP4C |
| Polycystin- | |
| 1_multiprotein_complex_(ACTN1,_CDH1,_ | SRC |
| SRC,_JUP,_VCL,_CTNNB1,_PXN,_BCAR1,_ | |
| PKD1,_PTK2,_TLN1) | |
| Phosphorylase_kinase_cornplex | CALM1 |
| Phosphatidylinositol_3-kinase (PIK3CA,_PIK3R1) | PIK3CA |
| Paf_complex | PAF1 |
| PU.1-SIN3A-HDAC_complex | SIN3A |
| PSF-p54(nrb)_complex | SFPQ |
| PRIMTl_complex | PRMT1 |
| PPP4C-PPP4R2-Gernin3- | PPP4C |
| Gemin4_complex | |
| POLR2A-CCNT1-CDK9-NCL-LEM6- | PPARGC1A |
| CPSF2_complex | |
| PLC-gamma-2-SLP-76-Lyn- | LYN |
| Grb2 complex _ | |
| PLC-gamma-2-Lyn-FcR-gamma_complex | LYN |
| PKA_(RII-alpha_and_RII-beta)-AKAP5- | |
| ADRBl_cornplex | PRKAR2B |
| PGC-1-SRp4O-SRp55-SRp75_complex | PPARGC1A |
| PCNA_complex | CDK4 |
| PCNA-DNA_polyrnerase_delta_complex | POLD1 |
| P53-BARD1-Ku70_complex | BARD1 |
| OCT2-TLE4_complex | TLE4 |
| NuRD.1_complex | M BD3 |
| Neddylin_ligaseiFBX011,_SKP1,_CUL1, | |
| _RBX1) | SKP1 |
| NK-3-Groucho-HIPK2-SIN3A-RbpA48- | |
| HDAC1_complex | SIN3A |
| NDPKA-AMPKalphal_complex | NME1 |
| NCOR_complex | NCOR1 |
| NCOR-SIN3-HDAC1_complex_ | SIN3A |
| NCOR-HDAC3_complex_ | NCOR1 |
| Mi2/NuRD-BCL6-MTA3_complex | MBD3 |
| MeCP2-SIN3A-HDAC_complex | SIN3A |
| MTA1_complex | MBD3 |
| MSL_complex_ | MSL3 |
| MRN-IRRAP_cornplex_MRE11A- | |
| RAD5O-NBN-TRRAP_complex _ | TRRAP |
| MGC1-DNA-PKcs-Ku_complex | PRKDC |
| MEP5O-PRMT5-ICLN_complex | CLNS1A |
| MCM8-ORC2-CDC6_complex | CDC6 |
| MBD1-Suy391-11-HP1_complex _ | MBD1 |
| MAP2K1-BRAF-RAF1-YWHAE- | |
| KSR1_complex | YWHAE |
| MAK-ACTR-AR_complex | NCOA3 |
| MAD1-mSin3A-HDAC2_complex_ | SIN3A |
| Kinase_maturation_complex_2 | TBK1 |
| Kaiso-NCOR_complex | NCOR1 |
| JBP1-pICIn_complex | CLNS1A |
| ITGAV-ITGB3-SLC3A2_complex_ | SLC3A2 |
| ITGA2b-ITGB3-CD47-SRC_complex | SRC |
| ING5_complex | IVIEAF6 |
| IKK-alpha--ER-alpha-AIB1_complex | NCOA3 |
| IGF1R-CXCR4-GNAI2-GNB1_complex | IGF1R |
| HuCHRAC_complex | BAZ1A |
| HUIC_complex | BARD1 |
| HIVIGB1-HMGB2-FISC70-ERP60- | |
| GAPDH_complex | GAPDH |
| HESl_promoter_corepressor_wmplex | CREBBP |
| HES1_promoter- | |
| Notch_enhancer_complex | SUPT16H |
| HERP1/HEY2-NCOR-SIN3A_complex | SIN3A |
| H2AX_complexi | PARP1 |
| GAIT complex | GAPDH |
| FOXO3-CBP_complex | CREBBP |
| FOXO1-FHL2-SIRT1_complex | FOX01 |
| FGFR2-c-Cbl-Lyn-Fyn_complex | LYN |
| FGFR1c-KLOcomplex | FGFR1 |
| FGF23-FGFR1c-KL_complex | FGFR1 |
| FGF21-FGFR1c-KLB_complex | FGFR1 |
| FE65-TSHZ3-HDACl_complex | TSHZ3 |
| FIFO-ATP_synthase,_mitochondrial | ATP5F1C |
| Ezh2_methyltransferase_complex,_cytosolic | EED |
| Emerin_cornplex_32 | SMARCB1 |
| Emerin_complex_24 | SAP130 |
| Elongator_holo_complex | ELP2 |
| Ecsit_complex_( ECSIT,_MT- | |
| 0O2,_GAPDH,_TRAF6,_NDUFAF1) | GAPDH |
| ETS2-SMARCA4-lNI1 complex _ | SMARCB1 |
| ESR1-RELA-BCL3-NCOA3_complex | NCOA3 |
| ERBB3-SPG1_complex | ERBB3 |
| DSSi_complex | BRCA2 |
| DRD4-KLHL12-CUL3_complex | CUL3 |
| DNTTIP1-ZNF541-HDAC1- | |
| HDAC2_complex | ZNF541 |
| DNMT3B_complex | SIN3A |
| DNA_synthesome_complex_(17_subunits) | POLD1 |
| DNA-PK-Ku_complex | PRKDC |
| DNA-PK-Ku-el F2-NF90-NF45_complex | PRKDC |
| DHX9-ADAR-vigilin-DNA-PK- | |
| Ku_antigen_complex | PRKDC |
| DA_complex | TAF3 |
| DAXX-MDM2-USP7_complex | USP7 |
| DAB_complex | TAR |
| Cytochrome_c_oxidase,_mitochondrial | COX411 |
| Condensini-PARP-1-XRCC1_complex | PARP1 |
| Cell_cycle_kinase_complex_CDK4 | CDK4 |
| CUL4A-DDB1-RBBP5_complex | RBBP5 |
| CUL4A-DDB1- EED_complex | EED |
| CS-MAP3K71P1-MAP3K7IP2_complex | CS |
| CREBBP-SNIAD3_hexameric_complex | CREBBP |
| CREBBP-SMAD3- | CREBBP |
| SMAD4_pentameric_complex | |
| CREBBP-SMADLhexameric_complex | CREBBP |
| CREBBP-SMAD2- | |
| SMAD4_pentameric...complex | CREBBP |
| CREBBP-KAT2B-MY0D1_complex | CREBBP |
| CNK1-SRC-RAF1_complex | SRC |
| CHTOP-methylosome_complex | PRMT1 |
| CF_IlAm_complex_(Cleavage_factor_11A | |
| m_complex) | SFPQ |
| CEP164-TTBK2_complex | TTBK2 |
| CDC7-DBF7 complex _ | CDC7 |
| CD98-LAT2-ITGB1_complex | SLC3A2 |
| CD20-1_CK-LYN-FYN- | |
| p75/80_complex,_(Raji_human_B_cell_line) | LYN |
| CCND3-CDK6_complex | CDK6 |
| CCND3-CDK4_complex | CDK4 |
| CCND2-CDK6_complex | CDK6 |
| CCND2-CDK4_complex | CDK4 |
| CCND1-CDK6_complex | CDK6 |
| CCND1-CDK4_complex | CDK4 |
| CCDC22-COMMD8-CUL3_complex | CUL3 |
| CBP-RARA-RXRA- | |
| DNA_complex,_ligand_stimu ed | CREBBP |
| CAS-SRC-FAK_complex | SRC |
| CAND1-CUL3-RBX1_complex | CUL3 |
| CALM1-_ | CALM1 |
| KCNQ4(splice variant_2)_complex | |
| CALM1- | |
| KCNQ4(splice_variant_1)_complex | CALM1 |
| BRCC complex | BRCA2 |
| BRCA1_C_complex | BARD1 |
| BRCA1_B_complex | BARD1 |
| BRCA1_A_complex | BARD1 |
| BRCA1-IRIS-pre-replication_complex | CDC6 |
| BRCA1-BARD1-UbcH7c_complex | BARD1 |
| BRCA1-BARD1-UbcH5c_complex | BARD1 |
| BRCA1-BARD1-POLR2A_complex | BARD1 |
| BRCA1-BARD1-BACH1- | BARD1 |
| DNA_damage_complex_II | |
| BRCAl-BARD1-BACH1- | BARD1 |
| DNA_damage_complex_I | |
| BRAF53-BRCA2_complex | BRCA2 |
| BRAF-RAFI-14-3-3_complex | YWHAZ |
| BRAF-MAP2K1-MAP2K2- | |
| YWHAE_complex | YWHAE |
| BARD1-BRCA1-CSIF_complex | BARD1 |
| BARD1-BRCA1-CSTF64_complex | BARD1 |
| Artemis-DNA-PK_complex | PRKDC |
| Anti-Sm_protein_complex | CLNSIA |
| ASF1-histone_containing_complex | CHEK2 |
| ARC_complex | ACAD8 |
| ANKS6-NEK8-INVS-NPHP3_complex | NPHP3 |
| AMY-1-S-AKAP84-RII-beta_complex | PRKAR2B |
| AJUBA-GF11-HDAC3_complex | GFI1 |
| AJUBA-GF11-HDAC2_complex | GFI1 |
| AJUBA-GF11-HDAC1_complex | GFI1 |
| 9b-1-1_complex | HUS1 |
| 9-1-1_complex | HUS1 |
| 944-RHINO_complex | HUS1 |
| 9-1-1-RAD17-RFC_complex | HUS1 |
| 9-1-1-POLB_complex | HUS1 |
| 9-1-1-LIG1_complex | HUS1 |
| 9-1-1-FEN1_complex | HUS1 |
| 9-1-1-APE1_complex | HUS1 |
| 6S_methyltransferase_complex | CLNS1A |
| 6S_methyltransferase_and_RG- | CLNSIA |
| containing_Sm_proteins_complex | |
| 60S_ribosomal_subunit_cytoplasmic | RPL27 |
| 5S-DNA-TFIIIA-TFIIIC2_subcomplex | GTF3C4 |
| 5S-DNA-TFIIIA-TFIIIC2-TFIIIB_subcomplex | GTF3C4 |
| 40S_ribosomal_subunit,_cytoplasmic | RPS4X |
| 20S_methyltransferase_core_complex | CLNS1A |
| 20S_methylosome_and_RG- | |
| containing_Sm_protein_complex | CLNS1A |
| 20S_methylosorne-SmD_complex | CLNS1A |
| 17S_LI2_snRNP | SRSF1 |

### Molecular Pathway Analysis:

To identify top molecular pathways enriched with multiple targets, the top targets were overlapped with KEGG pathway maps using the clusterProfiler R package. Top pathways are shown in Table 5 derived from hits identified using method 2.

TABLE 5
Molecular pathways associated with targets that upregulate HbF

| ID | Description | genelD | p. adjust | qvalue |
|---|---|---|---|---|
| hsa04922 | Glucagon | 32/207/801/808/816/817/818/1375/2538/ | 1.32E−08 | 7.39E−09 |
| | signaling | 92579/2645/160287/3945/441531/5563/ | | |
| | pathway | 5567/3276/5834 | | |
| hsa01200 | Carbon | 35/128/226/275/847/1431/1962/2597/2645/ | 5.10E−08 | 2.85E−08 |
| | metabolism | 3418/3421/5091/5095/441531/25796/ | | |
| | | 5631/8802/7167 | | |
| hsa04921 | Oxytocin | 107/113/114/115/801/808/57172/816/817/ | 8.08E−08 | 4.51E−08 |
| | signaling | 818/1026/29904/1956/5607/4638/85366/ | | |
| | pathway | 5563/5567/9475/6714 | | |
| hsa00010 | Glycolysis/ | 127/128/226/669/2538/92579/130589/2597/ | 5.57E−07 | 3.11E−07 |
| | Gluconeogenesis | 2645/160287/3945/441531/7167 | | |
| hsa01522 | Endocrine | 107/113/114/115/207/1026/1027/1956/ | 5.68E−07 | 3.18E−07 |
| | resistance | 3480/5600/5603/5291/5567/5925/6714 | | |
| hsa04912 | GnRH | 107/113/114/115/801/808/816/817/818/ | 2.16E−06 | 1.20E−06 |
| | signaling | 1956/5600/5603/5567/6714 | | |
| | pathway | | | |
| hsa04114 | Oocyte | 107/113/114/115/6790/801/808/816/817/ | 2.16E−06 | 1.20E−06 |
| | meiosis | 818/286151/3480/5567/6197/7531/7534 | | |
| hsa00071 | Fatty acid | 35/37/127/128/1375/1376/1579/10455/ | 2.69E−06 | 1.50E−06 |
| | degradation | 1962/2639 | | |
| hsa04750 | inflammatory | 107/113/114/115/801/808/816/817/818/ | 2.79E−06 | 1.56E−06 |
| | mediator | 5600/5603/5291/5567/6714 | | |
| | regulation | | | |
| | of TRP | | | |
| | channels | | | |
| hsa04015 | Rap1 | 107/113/114/115/207/801/808/1956/2260/ | 4.14E−06 | 2.31E−06 |
| | signaling | 2324/3480/3690/9223/9863/260425/5600/ | | |
| | pathway | 5603/5291/23683/6714 | | |
| hsa04971 | Gastric acid | 107/113/114/115/801/808/816/817/818/ | 6.06E−06 | 3.39E−06 |
| | secretion | 4638/85366/5567 | | |
| hsa04611 | Platelet | 107/113/114/115/207/3690/4067/5600/ | 7.03E−06 | 3.93E−06 |
| | activation | 5603/4638/85366/5291/5567/9475/6714 | | |
| hsa05214 | Glioma | 207/801/808/816/817/818/1026/1956/ | 2.29E−05 | 1.28E−05 |
| | | 3480/5291/5925 | | |
| hsa04722 | Neurotrophin | 207/27018/801/808/816/817/818/51135/ | 2.34E−05 | 1.31E−05 |
| | signaling | 5607/5600/5603/5291/6197/7531 | | |
| | pathway | | | |
| hsa01230 | Biosynthesis | 226/445/586/1431/2597/3418/3421/5091/ | 3.03E−05 | 1.69E−05 |
| | of amino | 441531/5631/7167 | | |
| | acids | | | |
| hsa00280 | Valine, | 27034/35/316/549/586/1962/11112/3157/ | 3.44E−05 | 1.92E−05 |
| | leucine and | 5095 | | |
| | isoleucine | | | |
| | degradation | | | |
| hsa04213 | Longevity | 107/113/114/115/207/847/3480/5291/5563/ | 3.71E−05 | 2.07E−05 |
| | regulating | 5567 | | |
| | pathway- | | | |
| | multiple | | | |
| | species | | | |
| hsa04925 | Aldosterone | 107/113/114/115/801/808/57172/816/817/ | 5.60E−05 | 3.13E−05 |
| | synthesis | 818/5567/23683 | | |
| | and | | | |
| | secretion | | | |
| hsa04914 | Progesterone- | 107/113/114/115/207/6790/3480/5600/ | 7.36E−05 | 4.11E−05 |
| | mediated | 5603/5291/5567/6197 | | |
| | oocyte | | | |
| | maturation | | | |
| hsa04066 | HIF-1 | 207/226/816/817/818/1026/1027/1956/ | 7.77E−05 | 4.34E−05 |
| | signaling | 2597/3480/5163/5291 | | |
| | pathway | | | |
| hsa04012 | ErbB | 207/816/817/818/1026/1027/1956/2065/ | 8.70E−05 | 4.86E−05 |
| | signaling | 57144/5291/6714 | | |
| | pathway | | | |
| hsa04714 | Thermogenesis | 107/113/114/115/8289/509/1375/1376/ | 0.000164 951 | 9.22E−05 |
| | | 2260/51780/5600/5603/5563/5567/6197/ | | |
| | | 6598/6599/7384 | | |
| hsa04068 | FoxO | 207/847/1026/1027/1956/2538/92579/ | 0.000246041 | 0.000137489 |
| | signaling | 3480/5600/5603/5291/5563/3276 | | |
| | pathway | | | |
| hsa05230 | Central | 207/1956/2260/2322/2645/5163/441531/ | 0.000302 864 | 0.000169242 |
| | carbon | 5291/23410 | | |
| | metabolism | | | |
| | in cancer | | | |
| hsa04720 | Long-term | 107/114/801/808/816/817/818/5567/6197 | 0.000364175 | 0.000203503 |
| | potentiation | | | |
| hsa05205 | Proteoglycans | 207/816/817/818/1026/1956/2065/2260/ | 0.000364175 | 0.000203503 |
| | in cancer | 3480/3690/5600/5603/5291/5567/9475/ | | |
| | | 6714 | | |
| hsa04020 | Calcium | 107/113/114/115/801/808/816/817/818/ | 0.000412694 | 0.000230615 |
| | signaling | 1956/2065/80271/4638/85366/5567 | | |
| | pathway | | | |
| hsa04261 | Adrenergic | 107/113/114/115/207/801/808/816/817/ | 0.000508051 | 0.000283901 |
| | signaling in | 818/5600/5603/5567 | | |
| | cardiomyocytes | | | |
| hsa04931 | Insulin | 32/207/1375/2538/92579/5291/5563/5834/ | 0.00055397 | 0.000309561 |
| | resistance | 6197/10998/57761 | | |
| hsa04211 | Longevity | 107/113/114/115/207/847/3480/5291/ | 0.00055397 | 0.000309561 |
| | regulating | 5563/5567 | | |
| | pathway | | | |
| hsa04973 | Carbohydrate | 207/2538/92579/8972/5291/6518/6523 | 0.0007462 | 0.00041698 |
| | digestion | | | |
| | and | | | |
| | absorption | | | |
| hsa00640 | Propanoate | 32/1962/160287/3945/5095/8802 | 0.000899319 | 0.000502544 |
| | metabolism | | | |
| hsa04713 | Circadian | 107/113/114/115/801/808/816/817/818/ | 0.000960425 | 0.00053669 |
| | entrainment | 5567 | | |
| hsa04910 | Insulin | 32/207/801/808/2538/92579/2645/5291/ | 0.001081413 | 0.000604298 |
| | signaling | 5563/5567/5577/5834 | | |
| | pathway | | | |
| hsa01212 | Fatty acid | 35/37/1375/1376/1962/3992/27349 | 0.001167221 | 0.000652248 |
| | metabolism | | | |
| hsa05418 | Fluid shear | 207/445/801/808/3690/5607/5600/5603/ | 0.001172205 | 0.000655034 |
| | stress and | 4258/5291/5563/6714 | | |
| | atherosclerosis | | | |
| hsa04916 | Melanagenesis | 107/113/114/115/801/808/816/817/818/ | 0.001309791 | 0.000731917 |
| | | 5567 | | |
| hsa04270 | Vascular | 107/113/114/115/801/808/1579/4638/ | 0.001317487 | 0.000736218 |
| | smooth | 85366/5567/9475 | | |
| | muscle | | | |
| | contraction | | | |
| hsa04911 | Insulin | 107/113/114/115/816/817/818/2645/5567 | 0.001562467 | 0.000873113 |
| | secretion | | | |
| hsa04923 | Regulation | 107/113/114/115/207/5291/5567 | 0.002165171 | 0.001209907 |
| | of lipolysis | | | |
| | in | | | |
| | adipocytes | | | |
| hsa04926 | Relaxin | 107/113/114/115/207/1956/5600/5603/ | 0.002287353 | 0.001278183 |
| | signaling | 5291/5567/6714 | | |
| | pathway | | | |
| hsa04024 | cAMP | 107/113/114/115/207/801/808/816/817/ | 0.002397555 | 0.001339764 |
| | signaling | 818/2867/5291/5567/9475 | | |
| | pathway | | | |
| hsa00480 | Glutathione | 2729/2880/257202/3418/4258/6241/51060 | 0.00248655 | 0.001389495 |
| | metabolism | | | |
| hsa04934 | Cushing's | 107/113/114/115/816/817/818/1026/1027/ | 0.00248655 | 0.001389495 |
| | syndrome | 1956/5567/5925 | | |
| hsa04725 | Cholinergic | 107/113/114/115/207/816/817/818/5291/ | 0.002502724 | 0.001398533 |
| | synapse | 5567 | | |
| hsa00650 | Butanoate | 35/622/56898/1962/3157 | 0.003003088 | 0.001678139 |
| | metabolism | | | |
| hsa04371 | Apelin | 107/113/114/115/207/801/808/4638/85366/ | 0.003058932 | 0.001709345 |
| | signaling | 556315567 | | |
| | pathway | | | |
| hsa04915 | Estrogen | 107/113/114/115/207/801/808/1956/5291/ | 0.003058932 | 0.001709345 |
| | signaling | 5567/6714 | | |
| | pathway | | | |
| hsa00310 | Lysine | 1962/2146/2639158508/93166/9739/9869 | 0.003065186 | 0.001712839 |
| | degradation | | | |
| hsa05215 | Prostate | 207/1026/1027/1956/2260/3480/3645/ | 0.003271725 | 0.001828254 |
| | cancer | 5291/5925 | | |
| hsa00020 | Citrate cycle | 1431/3418/3421/5091/8802 | 0.003771776 | 0.002107685 |
| | (TCA cycle) | | | |
| hsa00270 | Cysteine | 262/586/1786/2729/160287/3945 | 0.003799784 | 0.002123336 |
| | and | | | |
| | methionine | | | |
| | metabolism | | | |
| hsa04152 | AMPK | 32/207/1375/29904/2538/92579/3480/ | 0.003914111 | 0.002187222 |
| | signaling | 5210/5291/5563 | | |
| | pathway | | | |
| hsa01210 | 2- | 586/1431/3418/3421 | 0.003949829 | 0.002207182 |
| | Oxocarboxylic | | | |
| | acid | | | |
| | metabolism | | | |
| hsa00052 | Galactose | 2538/92579/130589/2645/8972 | 0.004086582 | 0.0022836 |
| | metabolism | | | |
| hsa04913 | Ovarian | 107/113/114/115/3480/5567 | 0.005577474 | 0.003116717 |
| | steroidogenesis | | | |
| hsa04540 | Gap | 107/113/114/115/1956/5607/5567/6714 | 0.006503423 | 0.003634141 |
| | junction | | | |
| hsa00072 | Synthesis | 622/56898/3157 | 0.006781885 | 0.003789748 |
| | and | | | |
| | degradation | | | |
| | of ketone | | | |
| | bodies | | | |
| hsa00500 | Starch and | 2538/92579/2645/8972/5834 | 0.00758873 | 0.004240616 |
| | sucrose | | | |
| | metabolism | | | |
| hsa04976 | Bile | 107/113/114/115/5567/10998/6523 | 0.00758873 | 0.004240616 |
| | secretion | | | |
| hsa05218 | Melanoma | 207/1026/1956/2260/3480/5291/5925 | 0.008097086 | 0.004524688 |
| hsa04918 | Thyroid | 107/113/114/115/2880/257202/5567 | 0.00933362 | 0.005215668 |
| | hormone | | | |
| | synthesis | | | |

### Consistency Across Two Different CRISPR Libraries:

To gain more confidence on the identified targets, an additional CRISPR library (library 2) with different set of genes and corresponding gRNAs was used. Only the HbF+ and FACs input samples were sequenced with library 2. Hits in library 2 were identified using method 2 (cutoff changed to 1.0) and without the dropout filter. Using this approach, a total of 209 hits were identified (FIG. 61B). Several common hits were identified in both libraries (FIG. 5B and Table 6).

TABLE 6
Hits identified using independent CRISPR libraries

| Gene Name | Uniprot ID | Description |
|---|---|---|
| TIC2 | O00142 | thymidine kinase 2, mitochondrial |
| HIST1H1B | P16401 | histone cluster 1 H1 family member b |
| BMX | P51813 | BMX non-receptor tyrosine kinase |
| G6PC3 | Q9BUM1 | glucose-6-phosphatase catalytic subunit 3 |
| IDH3G | P51553 | isocitrate dehydrogenase 3 (NAD(+)) gamma |
| PRPS1 | P60891 | phosphoribosyl pyrophosphate synthetase 1 |
| PDK3 | Q15120 | pyruvate dehydrogenase kinase 3 |
| MBD3 | O95983 | methyl-CpG binding domain protein 3 |
| TYRO3 | Q06418 | TYRO3 protein tyrosine kinase |
| EPHA5 | P54756 | EPH receptor A5 |
| BDH2 | Q9BUT1 | 3-hydroxybutyrate dehydrogenase 2 |
| CDKN1B | Q6I9V6 | cyclin dependent kinase inhibitor 1B |
| PRMT2 | P55345 | protein arginine methyltransferase 2 |
| MAP4K4 | O95819 | mitogen-activated protein kinase kinase |
| | | kinase kinase 4 |
| INO80C | Q6P198 | INO80 complex subunit C |
| SRSF3 | P84103 | serine and arginine rich splicing factor 3 |
| ADCY7 | P51828 | adenylate cyclase 7 |
| TADA1 | Q96BN2 | transcriptional adaptor 1 |
| IKZF1 | R9R4D9 | 1KAROS family zinc finger 1 |
| PARP1 | P09874 | poly(ADP-ribose) polymerase 1 |
| PKN3 | Q6P5Z2 | protein kinase N3 |
| MVK | Q03426 | mevalonate kinase |
| CTBP1 | X5D8Y5 | C-terminal binding protein 1 |
| CUL4A | Q13619 | cullin 4A |
| AKT1 | P31749 | AKT serine/threonine kinase 1 |
| GLYR1 | | glyoxylate reductase 1 homolog |
| ACAD8 | Q9UKU7 | acyl-CoA dehydrogenase family member 8 |

### Expression Specificity of Hits in Blood Tissue and Erythroid Lineage:

Hits identified using method 2 were prioritized based on their expression in blood tissue, relevant to SCD. This was performed using GTEx gene expression data from 15,598 samples across 31 different tissues (The GTEx Consortium Nature Genetics). A mean Z-score was calculated to identify genes with high blood specific expression. The blood Z-scores for hits were calculated as follows:

$${Z}_{g , blood} = {mean}_{i ∈ blood} ( \frac{{g}_{i} - {μ}_{g}}{{σ}_{g}} )$$

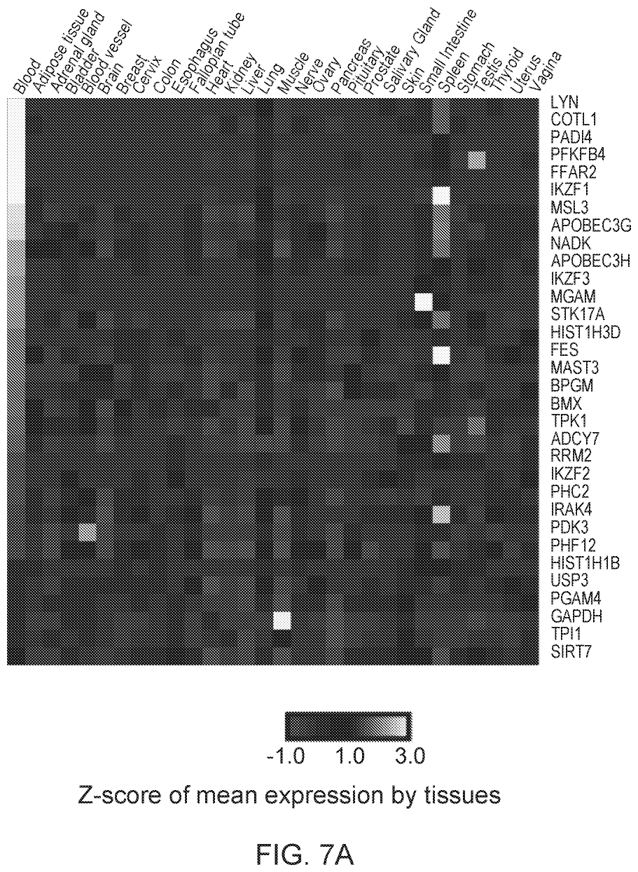

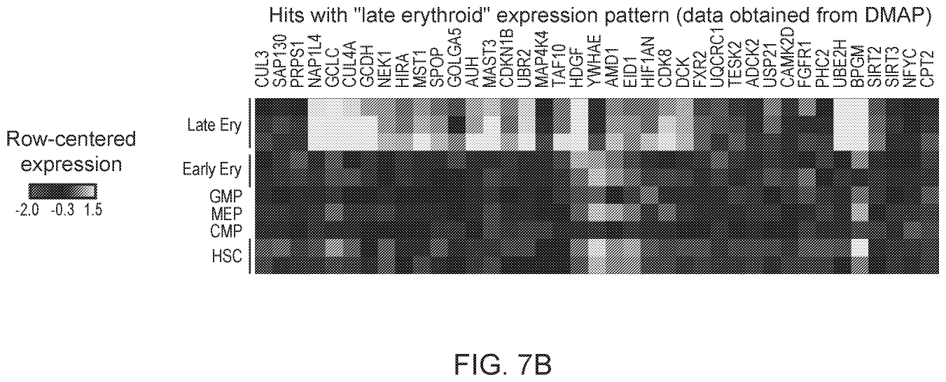

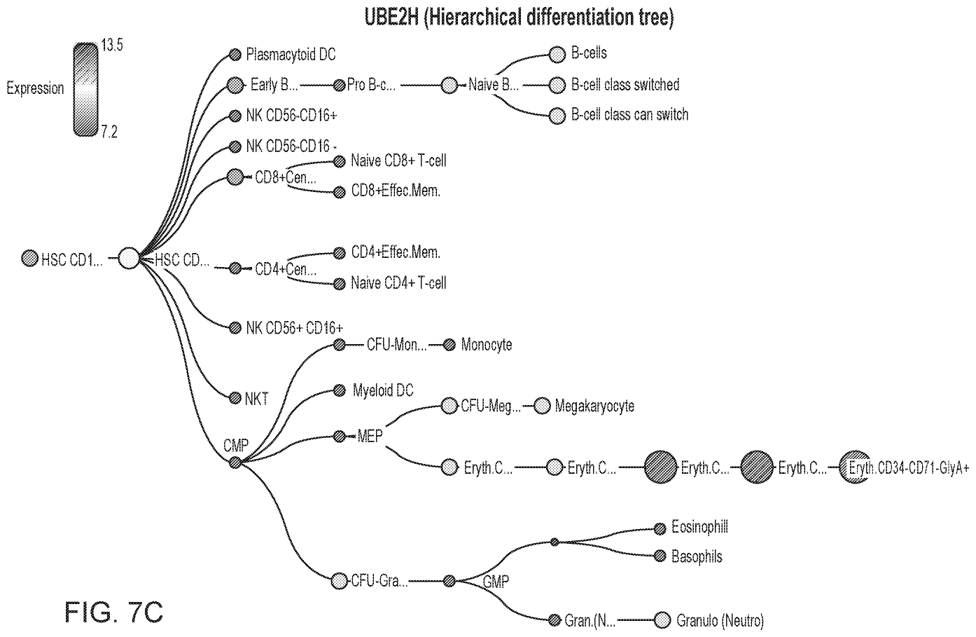

In the above equation, Z_{g,blood} is the mean Z-score of gene “g” in blood tissue, gᵢ is the expression of gene “g” in sample “i”, μ_{g} is the mean expression of gene “g” across all samples, and σ_{g}, is the standard deviation of gene “g” across all samples. In total, 32 hits were identified that had a Z_{g,blood} greater than 1 (FIG. 7A and Table 7).

TABLE 7
Additional drug targets identified using blood-specific network

| Gene | Uniprot | | |
|---|---|---|---|
| Name | ID | Description | Blood_mean_Zscore |
| PGAM4 | Q8N0Y7 | phosphoglycerate mutase family member 4 | 1.165971631 |
| IKZF2 | Q9UKS7 | IKAROS family zinc finger 2 | 1.549012532 |
| USP3 | Q9Y6I4 | ubiquitin specific peptidase 3 | 1.198035702 |
| MSL3 | Q8N5Y2 | MSL complex subunit 3 | 2.809489699 |
| HIST1H1B | P16401 | histone cluster 1 H1 family member b | 1.266391878 |
| BMX | P51813 | BMX non-receptor tyrosine kinase | 1.82329169 |
| NADK | O95544 | NAD kinase | 2.357039301 |
| HIST1H3D | P68431 | histone cluster 1 H3 family member d | 1.940003256 |
| PADA | Q9UM07 | peptidyl arginine deiminase 4 | 3.284882803 |
| RRM2 | P31350 | ribonucleotide reductase regulatory subunit | 1.58105877 |
| | | M2 | |
| TPI1 | V9HWK1 | triosephosphate isomerase 1 | 1.110545454 |
| PDK3 | Q15120 | pyruvate dehydrogenase kinase 3 | 1.461996437 |
| PFKFB4 | Q66535 | 6-phosphofructo-2-kinase/fructose-2,6- | 3.170252799 |
| | | biphosphatase 4 | |
| COTL1 | Q14019 | coactosin like F-actin binding protein 1 | 3.522557555 |
| LYN | P07948 | LYN proto-oncogene, Src family tyrosine kinase | 3.60867428 |
| MGAM | O43451 | maltase-glucoamylase | 2.203722836 |
| PHF12 | Q96QT6 | PHD finger protein 12 | 1.445134764 |
| SIRT7 | Q9NRC8 | sirtuin 7 | 1.011603642 |
| PHC2 | Q8IXK0 | polyhomeotic homolog 2 | 1.528946092 |
| FFAR2 | O15552 | free fatty acid receptor 2 | 3.013584729 |
| FES | P07332 | FES proto-oncogene, tyrosine kinase | 1.938512739 |
| ADCY7 | P51828 | adenylate cyclase 7 | 1.667462363 |
| IKZF3 | Q9UKT9 | IKAROS family zinc finger 3 | 2.223300296 |
| IKZE1 | R9R4D9 | IKAROS family zinc finger 1 | 2.970394101 |
| TPK1 | Q9H3S4 | thiamin pyrophosphokinase 1 | 1.798433907 |
| STK17A | Q9UEE5 | serine/threonine kinase 17a | 2.137292947 |
| APOBEC3G | Q9HC16 | apolipoprotein B mRNA editing enzyme | 2.766529254 |
| | | catalytic subunit 3G | |
| APOBEC3H | M4W6S4 | apolipoprotein B mRNA editing enzyme | 2.353495477 |
| | | catalytic subunit 3H | |
| MAST3 | O60307 | microtubule associated serine/threonine kinase | 1.933987547 |
| | | 3 | |
| IRAK4 | Q9NWZ3 | interleukin 1 receptor associated kinase 4 | 1.511622129 |
| GAPDH | V9HVZ4 | glyceraldehyde-3-phosphate dehydrogenase | 1.124617068 |
| BPGM | P07738 | bisphosphoglycerate mutase | 1.876857003 |

Blood tissue is heterogeneous with many different cell-types, which are not all relevant to SCD. To focus on erythroid lineage, which is primarily affected in SCD, hits were overlapped with lineage specific modules identified by DMAP project (Novershtern et al, Cell). Many hits were identified that were expressed in progenitor and late erythroid lineages (Table 8) (FIGS. 7B and 7C).

TABLE 8
Hits with specific induction pattern in erythroid lineage

| Hit | Induction_pattern |
|---|---|
| AKT1 | Earlt Mye, T/B-cell and GRANs |
| ROCK2 | Earlt Mye, T/B-cell and GRANs |
| TTBK2 | Earlt Mye, T/B-cell and GRANs |
| TBK1 | Earlt Mye, T/B-cell and GRANs |
| SUCLG1 | Earlt Mye, T/B-cell and GRANs |
| TAF5L | Earlt Mye, T/B-cell and GRANs |
| PGLS | Earlt Mye, T/B-cell and GRANs |
| SETDB1 | Earlt Mye, T/B-cell and GRANs |
| ADCY7 | Earlt Mye, T/B-cell and GRANs |
| NAP1L1 | Earlt Mye, T/B-cell and GRANs |
| RPL27 | Earlt Mye, T/B-cell and GRANs |
| HMGN2 | Earlt Mye, T/B-cell and GRANs |
| DGUOK | Earlt Mye, T/B-cell and GRANs |
| SPEN | Earlt Mye, T/B-cell and GRANs |
| ARID4A | Earlt Mye, T/B-cell and GRANs |
| PRPF4B | Earlt Mye, T/B-cell and GRANs |
| MYBBP1A | Earlt Mye, T/B-cell and GRANs |
| FBL | Earlt Mye, T/B-cell and GRANs |
| PARP1 | Earlt Mye, T/B-cell and GRANs |
| ADH5 | Earlt Mye, T/B-cell and GRANs |
| SMARCC1 | Earlt Mye, T/B-cell and GRANs |
| CTBP1 | Earlt Mye, T/B-cell and GRANs |
| EXOSC9 | Earlt Mye, T/B-cell and GRANs |
| ARID1A | Earlt Mye, T/B-cell and GRANs |
| MTF2 | Earlt Mye, T/B-cell and GRANs |
| PRKDC | Earlt Mye, T/B-cell and GRANs |
| RNF8 | Earlt Mye, T/B-cell and GRANs |
| YEATS2 | Earlt Mye, T/B-cell and GRANs |
| ACACB | Earlt Mye, T/B-cell and GRANs |
| LDHB | Earlt Mye, T/B-cell and GRANs |
| PRKACB | Earlt Mye, T/B-cell and GRANs |
| BDH2 | Earlt Mye, T/B-cell and GRANs |
| PRKD3 | Earlt Mye, T/B-cell and GRANs |
| HMG20A | Earlt Mye, T/B-cell and GRANs |
| PIK3C2A | Earlt Mye, T/B-cell and GRANs |
| CHD1 | Earlt Mye, T/B-cell and GRANs |
| SRP72 | Earlt Mye, T/B-cell and GRANs |
| CS | Earlt Mye, T/B-cell and GRANs |
| HLTF | Earlt Mye, T/B-cell and GRANs |
| NASP | Earlt Mye, T/B-cell and GRANs |
| HMGCS1 | Earlt Mye, T/B-cell and GRANs |
| EHHADH | HSC, Early Mye |
| MAGI2 | HSC, Early Mye |
| HIST1H3D | HSC, Early Mye |
| EZH2 | HSC, Early Mye |
| NME7 | HSC, Early Mye |
| IKZF2 | HSC, Early Mye |
| IGF1R | HSC, Early Mye |
| IDH2 | HSC, Early Mye |
| SSRP1 | HSC, Early Mye |
| DTYMK | HSC, Early Mye |
| GAPDH | HSC, Early Mye |
| PCCA | HSC, Early Mye |
| ALDOA | HSC, Early Mye |
| USP46 | HSC, Early Mye |
| TPI1 | HSC, Early Mye |
| PIK3CB | HSC, Early Mye |
| G6PC3 | HSC, Early Mye |
| MGST2 | HSC, Early Mye |
| FLT3 | HSC, Early Mye |
| CDKN1C | HSC, Early Mye |
| MYLK | HSC, Early Mye |
| BCAT1 | HSC, Early Mye |
| SMARCA1 | HSC, Early Mye |
| FADS1 | HSC, Early Mye |
| CUL3 | Late ERY, T/B-cell and GRANs |
| SAP130 | Late ERY, T/B-cell and GRANs |
| PRPS1 | Late ERY, T/B-cell and GRANs |
| NAP1L4 | Late ERY, T/B-cell and GRANs |
| GCLC | Late ERY, T/B-cell and GRANs |
| CUL4A | Late ERY, T/B-cell and GRANs |
| GCDH | Late ERY, T/B-cell and GRANs |
| NEK1 | Late ERY, T/B-cell and GRANs |
| HIRA | Late ERY, T/B-cell and GRANs |
| MST1 | Late ERY, T/B-cell and GRANs |
| SPOP | Late ERY, T/B-cell and GRANs |
| GOLGA5 | Late ERY, T/B-cell and GRANs |
| AUH | Late ERY, T/B-cell and GRANs |
| MAST3 | Late ERY, T/B-cell and GRANs |
| CDKN1B | Late ERY, T/B-cell and GRANs |
| UBR2 | Late ERY, T/B-cell and GRANs |
| MAP4K4 | Late ERY, T/B-cell and GRANs |
| TAF10 | Late ERY, T/B-cell and GRANs |
| HDGF | Late ERY, T/B-cell and GRANs |
| YWHAE | Late ERY, T/B-cell and GRANs |
| AMD1 | Late ERY, T/B-cell and GRANs |
| EID1 | Late ERY, T/B-cell and GRANs |
| HIF1AN | Late ERY, T/B-cell and GRANs |
| CDK8 | Late ERY, T/B-cell and GRANs |
| DCK | Late ERY, T/B-cell and GRANs |
| FXR2 | Late ERY, T/B-cell and GRANs |
| UQCRC1 | Late ERY, T/B-cell and GRANs |
| TESK2 | Late ERY, T/B-cell and GRANs |
| ADCK2 | Late ERY, T/B-cell and GRANs |
| USP21 | Late ERY, T/B-cell and GRANs |
| CAMK2D | Late ERY, T/B-cell and GRANs |
| FGFR1 | Late ERY, T/B-cell and GRANs |
| PHC2 | Late ERY |
| UBE2H | Late ERY |
| BPGM | Late ERY |
| SIRT2 | Late ERY |
| SIRT3 | Late ERY |
| NFYC | Late ERY |
| CPT2 | Late ERY |
| ITGB3 | MYE |
| AURKA | MYE |
| RRM2 | MYE |
| PRKAR2B | MYE |
| TOP2A | MYE |
| WRB | MYE |
| CAT | MYE |
| RMI1 | MYE |

Table 9 provides a list of various components of complexes and pathways identified herein as targets for increasing expression of HbF. Any of these may be targeted according to any of the methods disclosed herein.

TABLE 9
Complexes associated with hits and the other complex subunits within hits

| ComplexName | hit_members | other_members |
|---|---|---|
| ALL-1 | SIN3A; MBD3; | SAP18; CHD3; WDR5; KDM1A; HDAC1; HDAC2; KMT2A; |
| supercornplex | SMARCB1; SMARCC1; | CPSF2; RAN; RBBP4; RBBP5; RBBP7; SMARCA2; SMARCC2; |
| | MTA2 | TAF1; TAF6; TAF9; TAF12; TBP; SYMPK; SMARCA5; |
| | | SAP30; EFTUD2 |
| Anti-HDAC2 | HMG20B; SIN3A; | CHD3; CHD4; KDM1A; RCOR1; GSE1; GTF2I; HDAC1; |
| complex | MTA2 | HDAC2; PHF21A; RBBP4; RBBP7; ZMYM2; MTA1; ZMYM3 |
| BAF complex | SMARCB1; SMARCC1; | ACTL6B; ARID1B; ACTB; SMARCA2; SMARCA4; SMARCC2; |
| | ARID1A | SMARCD1; SMARCE1; ACTG1; ACTL6A |
| BRG1-SIN3A | SIN3A; SMARCB1; | PRMT5; HDAC2; RBBP4; SMARCA4; SMARCC2; SMARCD1; |
| complex | SMARCC1; | SMARCD2; SMARCD3; SMARCE1; ACTL6A |
| | ARID1A | |
| BRM-SIN3A | SIN3A; SMARCB1; | PRMT5; HDAC1; HDAC2; RBBP4; SMARCA2; SMARCC2; |
| complex | SMARCC1; ARID1A | SMARCD1; SMARCD2; SMARCD3; SMARCE1; ACTL6A |
| BRM-SIN3A- | SIN3A; SMARCB1; | PRMTS; HDAC2; SMARCA2; SMARCC2; SMARCD1; |
| HDAC complex | SMARCC1; ARID1A | SMARCD2; SMARCE1; ACTL6A |
| EBAFa complex | SMARCB1; SMARCC1; | MLLT1; SMARCA4; SMARCC2; SMARCD1; SMARCD2; |
| | ARID1A | SMARCE1; ACTL6A |
| GCN5-TRRAP | TADA3; TAF5L; | KAT2A; MSH6; MSH2; BRCA1; TAF9; TRRAP; SUPT3H |
| histone | TAF10 | |
| acetyltransferase | | |
| complex | | |
| ING2 complex | SIN3A; ARID4A; | BRMS1; HDAC1; HDAC2; ING2; RBBP4; RBBP7; SUDS3; |
| | SAP130 | BRMS1L; SAP30 |
| Kinase | MAP2K5; YWHAE; | YWHAQ; CDC37; MARK2; HSPA4; HSP90AA1; HSP90AB1; |
| maturation | YWHAZ | MAP3K3; PFDN2; YWHAB; YWHAG; YWHAH; PDRG1; |
| complex 1 | | TRAF7 |
| LARC complex | | |
| (LCR-associated | MBD3; SMARCB1; | CHD4; HDAC1; HDAC2; HNRNPC; GATAD2B; RBBP4; |
| remodeling | SMARCC1; ARID1A; | DPF2; ACTB; SMARCA4; SMARCC2; SMARCD2; SMARCE1; |
| complex) | MTA2 | ACTL6A; MBD2 |
| LSD1 complex | HMG20B; HMG20A; | PHF21B; KDM1A; RCOR1; HDAC1; HSPA1A; HSPA1B; |
| | CTBP1 | PHF21A; RCOR3; RREB1; ZMYM2; ZNF217 |
| MTA2 complex | SIN3A; MBD3; MTA2 | CHD4; HDAC1; HDAC2; RBBP4; RBBP7 |
| NUMAC | SMARCB1; SMARCC1; | CARM1; SCYL1; ACTB; SMARCA4; SMARCC2; SMARCD1; |
| complex | ARID1A | SMARCE1 |
| (nucleosomal | | |
| methylation | | |
| activator | | |
| complex) | | |
| PCAF complex | TADA3; TAF6L; | TADA2A; TAF9; TAF12; TRRAP; SUPT3H; KAT2B |
| | TAF5L; TAF10 | |
| RNA | CDK8; SMARCB1; | DRAP1; CREBBP; ERCC3; GTF2B; GTF2E1; GTF2F1; GTF2H1; |
| polymerase II | SMARCC1 | GTF2H3; POLR2A; PCSK4; SMARCA2; SMARCA4; SMARCC2; |
| complex, | | SMARCD1; SMARCE1; TBP; ACTL6A; KAT2B; CCNC; |
| chromatin | | MED21 |
| structure | | |
| modifying | | |
| RNA | CDK8; SMARCB1; | GTF2F1; SMARCC2; CCNC; CCNH; MED21 |
| polymerase II | SMARCC1 | |
| complex, | | |
| incomplete | | |
| (CDK8 | | |
| complex), | | |
| chromatin | | |
| structure | | |
| modifying | | |
| SAGA complex, | TADA3; TAF6L; | ADA; SGF29; ATXN7L2; ATXN7L1; USP22; KAT2A; TAF9B; |
| GCN5-linked | TAF5L; ATXN7L3; | SUPT20H; TAF9; TAF12; TRRAP; SUPT3H; TADA2B; SUPT7L |
| | TAF10 | |
| SIN3-ING1b | SIN3A; SMARCB1; | SAP18; HDAC1; HDAC2; ING1; ARID4B; RBBP4; RBBP7; |
| complex II | SMARCC1; ARID1A | SMARCA4; SMARCC2; SMARCD1; ACTL6A; SAP30 |
| STAGA complex | TADA3; TAF6L; | SF3B3; KAT2A; ATXN7; TAF9; TAF12; TRRAP; SUPT3H; |
| | TADA1; TAF5L; | SUPT7L |
| | TAF10 | |
| STAGA | TADA3; TAF6L; | SGF29; USP22; KAT2A; SUPT20H; ENY2; ATXN7; TAF9; |
| complex, SPT3- | TADA1; TAF5L; | TAF12; TRRAP; SUPT3H; TADA2B; SUPT7L |
| linked | ATXN7L3; TAF10; | |
| | SAP130 | |
| SWI-SNF | SMARCB1; SMARCC1; | SMARCA2; SMARCA4; SMARCC2; SMARCD2; SMARCE1; |
| chromatin | ARID1A | BRCA1; ACTL6A |
| remodeling-. | | |
| related-BRCA1 | | |
| complex | | |
| TFTC complex | TADA3; TAF6L; | SF3B3; KAT2A; ATXN7; TAF2; TAF4; TAF5; TAF6; TAF7; |
| (TATA-binding | TAF5L; TAF10 | TAF9; TAF12; TAF13; TRRAP; SUPT3H |
| protein-free | | |
| TAF-II- | | |
| containing | | |
| complex) | | |
| USP22-SAGA | TADA3; ATXN7L3; | USP22; KAT2A; TAF9B; TRRAP; TADA2B |
| complex | TAF10 | |
| WINAC complex | SMARCB1; SMARCC1; | CHAF1A; SUPT16H; SMARCA2; SMARCA4; SMARCC2; |
| | ARID1A | SMARCD1; SMARCE1; TOP2B; VDR; ACTL6A; BAZ1B |
| p300-CBP- | SMARCB1; SMARCC1; | CREBBP; EP300; SMARCA4; SMARCC2 |
| p270-SWI/SNF | ARID1A | |
| complex | | |

### Example 4

### SPOP and CUL3 Genetic Validation in Primary CD34+ Cells

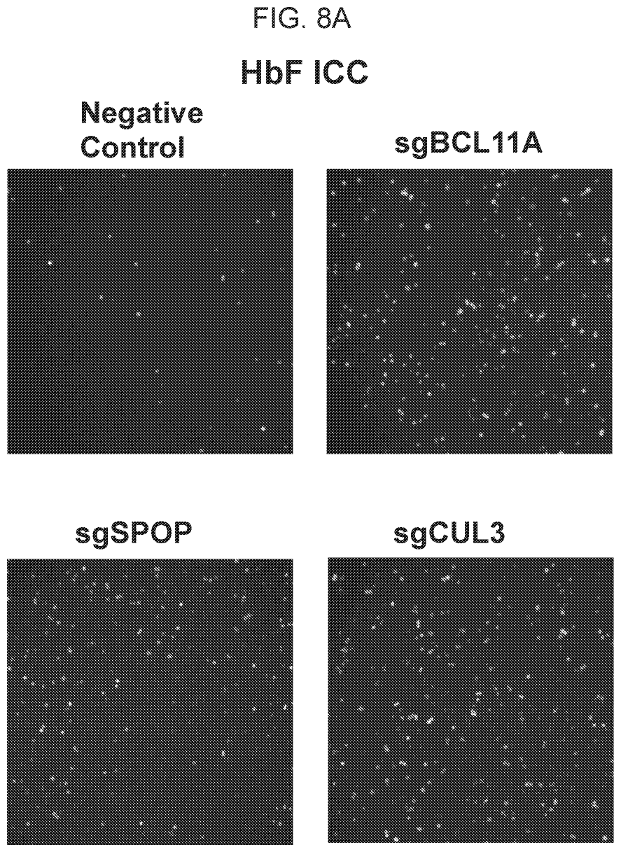

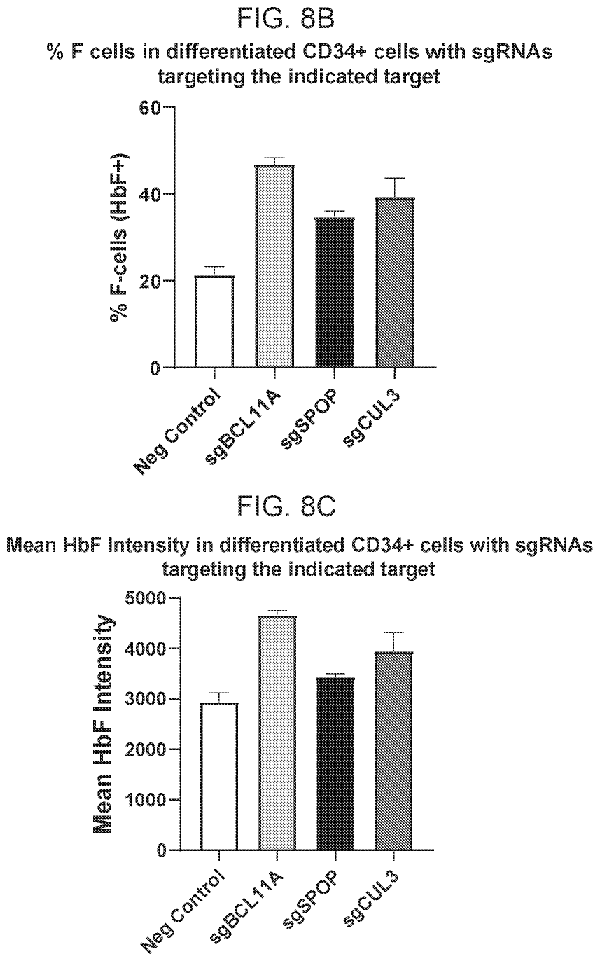

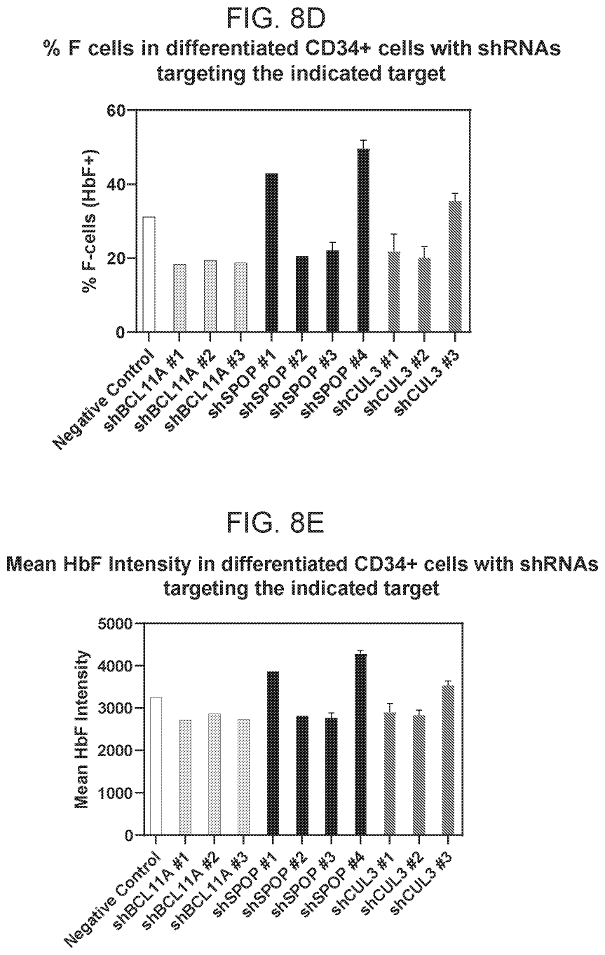

SPOP and CUL3 were identified using pooled CRISPR screening in the HUDEP2 model as regulators of fetal hemoglobin expression. To further investigate the role of SPOP and CUL3 in fetal hemoglobin regulation, primary CD34+ cells from a healthy donor were used with CRISPR Cas9- and shRNA-mediated genetic perturbation approaches. The impact on HbF levels was studied in differentiated CD34+ cells using HbF immunocytochemistry (ICC) (FIG. 8A).

HbF levels were determined by HbF ICC using CRISPR Cas9-RNP-based loss of function. Cas9-RNP complexes were electroporated into proliferating CD34+ cells. Cells were then differentiated for 7 days down the erythroid lineage and HbF levels were quantified using HbF ICC. Non-target guide RNAs were used as negative controls and guide RNAs targeting BCL11A were used as positive controls in this experimental design. Genetically perturbing SPOP and CUL3 using either CRISPR-Cas9 or shRNA led to elevated HbF levels, as measured by percent F cells within the population of differentiated erythroid cells or mean HbF levels per cell. The gRNAs used for SPOP were TAACTTTAGCTTTTGCCGGG (SEQ ID NO: 91), CGGGCATATAGGTTTGUGCA (SEQ ID NO: 92), GTTGCGAGTAAACCCCAAA (SEQ ID NO: 93) and the gRNAs used for CUL3 were GAGCATCTCAAACACAACGA (SEQ ID NO: 94), CGAGATCAAGTTGTACGTTA (SEQ ID NO: 95), TCATCTACGGCAAACTCTAT (SEQ ID NO: 96) using the CRISPR Cas9-RNA method via electroporation. The Cas9-gRNA complexes were made independently and the three complexes per target were pooled for the cellular assay. The shRNAs used for SPOP were CCGGCACAGATCAAGGTAGTGAAATCTCGAGATTTCACTACCTTGATCTGTGTTT TTTG (SPOP shRNA #2) (SEQ ID NO: 97), CCGGCAAGGTAGTGAAATTCTCCTACTCGAGTAGGAGAATTCACTACCTTGTTT TTTG (SPOP shRNA #4) (SEQ ID NO: 98), CCGGCAGATGAGTTAGGAGGACTGTCTCGAGACAGTCCTCCTAACTCATCTGTTT TTTG (SPOP shRNA #1) (SEQ ID NO: 99), and CCGGCACAAGGCTATCTTAGCAGCTCTCGAGAGCTGCTAAGATAGCCTTGTGTTT TTTG (SPOP shRNA #3) (SEQ ID NO: 100). The shRNAs used for CUL3 were CCGGGACTATATCCAGGGCTTATTGCTCGAGCAATAAGCCCTGGATATAGTCTTT TTG (CUL3 shRNA #1) (SEQ ID NO: 101), CCGGCGTAAGAATAACAGTGGTCTTCTCGAGAAGACCACTGTTATTCTTACGTTT TTG (CUL3 shRNA #3) (SEQ ID NO: 102), and CCGGCGTGTGCCAAATGGTTTGAAACTCGAGTTTCAAACCATTTGGCACACGTTT TTG (CUL3 shRNA #2) (SEQ ID NO: 103). HbF ICC allows for the quantification of percent F cell and HbF intensity on a per-cell basis. An F cell is an erythroid cell that has a detectable level of HbF beyond a defined threshold and the percent F cells is defined as the percent of cells among a population of cells that are defined as F cells. The percent F cells and mean HbF intensity cells were quantified for negative control, sgBCL11A, sgSPOP and sgCUL3. HbF levels determined by HbF ICC using shRNA-based loss of function. shRNA vectors were electroporated into proliferating CD34+ cells. Cells were then differentiated for 7 days down the erythroid lineage and HbF levels were quantified using ICC. The percent F cells (FIG. 8B and FIG. 8D) and mean HbF intensity (FIG. 8C and FIG. 8E) were quantified for individual shRNA constructs for negative control. shBCL11A, shSPOP and shCUL3.

### Methods

### Cell Culture

Human Mobilized Peripheral Blood Primary CD34+ cells were expanded from thaw by seeding 100,000 viable cells/mL in a culture flask containing CD34+ Phase 1 Media comprised of IMDM, 100 ng/mL hSCF, 5 ng/mL IL-3, 3 IU/mL EPO, 250 ug/mL transferrin, 2.5% normal human serum, 1% pen/strep, 10 ng/mL heparin, 10 ug/mL insulin. The cells were supplemented by adding an additional 1× culture volume of CD34+ Phase 1 Media on Day 3 after thaw. After 5 days of expansion, Primary CD34+ cells were transfected with RNP complex.

### Cas9-gRNA RNP Preparation and Nucleofection

TE buffer was used to resuspend lyophilized crRNA and tracrRNA. The crRNA and tracrRNA were added to annealing buffer and annealed in thermocycler. Multiple sgrRNAs per gene were pooled into a microcentrifuge tube. Each sgRNA was mixed with TrueCut Cas9 v2 and incubated for 10 minutes to generate RNP complex. After counting, 144,000 CD34+ cells were added to the transfection cuvette and combined with transfection solution (β3, RNP complex, glycerol). The cells were transfected using an Amaxa Nucleofector and then transferred to a 12-well plate with 1 mL of prewarmed Phase 1 media.

### In Vitro Differentiation

The day after transfection, the cells are supplemented with an additional 0.5 mL of Phase 1 media. On the 5th day post transfection the cells were differentiated towards erythroid lineage by complete medium exchange into CD34+ Phase 2 Media comprised of IMDM, 100 ng/mL hSCF, 5 ng/mL IL-3, 3 IU/mL EPO, 250 ug/mL transferrin, 2.5% normal human serum, 1% pen/strep, 10 ng/mL heparin, 10 ug/mL insulin. Two days after changing to Phase 2 media the cells were centrifuged, and 1 mL of Phase 2 media exchanged with fresh Phase 2 media. After another 2 days, the cells were harvested for HbF analysis by ICC.

### HbF ICC Protocol

To collect the CD34+ cells, 40 uL from each well were transferred to a 384-well plate in duplicate and the plate was centrifuged. First the plate was washed with 25 μL of PBS. Then the plate was fixed with 25 μL of 4% paraformaldehyde for 10 minutes at room temperature. The cells were then washed three times with 25 μL of PBS. Next the cells were permeabilized and blocked for 1 hour at room temperature in 25 μL of Perm/Block buffer comprised of 1×PBS, 1% bovine serum albumin, 10% fetal bovine serum, 0.3M glycine, and 0.1% tween-20. Then the cells were washed three times with 25 μL of 0.1% tween in PBS. After washing, the cells were incubated overnight at 4° C. with 25 μL of HbF-488 Primary Antibody (ThermoFisher MHFH01-4) diluted 1:40 in 0.1% tween and Hoescht diluted 1:2000 in 0.1% tween. The next day the cells were again washed three times with 25 μL of 0.1% tween in PBS and foil sealed for imaging on the ThermoFisher CellInsight CX7.

The plates were then scanned on the CX7 at 10× magnification, and 9 images were acquired per well. The software algorithm then identified nuclei and calculated a total nuclei count using the Hoechst staining on channel 1. After nuclei were identified, the algorithm calculated the average nuclear intensity of the HbF staining on channel 2.

### REFERENCES

### An International Effort to Cure a Global Health Problem: A Report on the 19th

### Hemoglobin Switching Conference.

Blobel G A, Bodine D, Brand M, Crispino J, de Bruijn M F, Nathan D, Papayannopoulou T, Porcher C, Strouboulis J. Zon L. Higgs D R, Stamatoyannopoulos G, Engel J D.
Exp Hematol. 2015 October; 43(10):821-37. doi: 10.1016/j.exphem.2015.06.008. Epub 2015 Jul. 2. Review. PMID:26143582

### Control of Globin Gene Expression During Development and Erythroid Differentiation.

Stamatoyannopoulos G.
Exp Hematol. 2005 March; 33(3):259-71. Review. PMID: 15730849

### Fetal Haemoglobin Induction in Sickle Cell Disease.

Paikari A, Sheehan V A.
Br J Haematol. 2018 January; 180(2):189-200. doi: 10.1111/bjh.15021. Epub 2017 Nov. 16. Review. PMID: 29143315

### Fetal Haemoglobin in Sickle-Cell Disease: From Genetic Epidemiology to New Therapeutic Strategies.

Lettre G, Bauer D E.
Lancet. 2016 Jun. 18; 387(10037):2554-64. doi: 10.1016/S0140-6736(15)01341-0. Review. PMID: 27353686

### Locus Control Regions.

Li Q. Peterson K R, Fang X, Stamatoyannopoulos G.
Blood. 2002 Nov. 1; 100(9):3077-86. Review. PMID: 12384402

### Pomalidomide and Lenalidomide Regulate Erythropoiesis and Fetal Hemoglobin Production in Human CD34+ Cells.

Moutouh-de Parseval L A, Verhelle D, Glezer E, Jensen-Pergakes K, Ferguson G D, Corral L G, Morris C L, Muller G. Brady H, Chan K.
J Clin Invest. 2008 January; 118(1):248-58. PMID: 18064299

### Augmentation of Fetal-Hemoglobin Production in Anemic Monkeys by Hydroxyurea.

Letvin N L, Linch D C, Beardsley G P, McIntyre K W, Nathan D G.
N Engl J Med. 1984 Apr. 5; 310(14):869-73. PMID: 619967

### EHMT1 and EHMT2 Inhibition Induces Fetal Hemoglobin Expression.

Renneville A, Van Galen P, Canver M C, McConkey M. Krill-Burger J M, Dorfman D M, Holson E B, Bernstein B E, Orkin S H, Bauer D E, Ebert B L.
Blood. 2015 Oct. 15; 126(16):1930-9. doi: 10.1182/blood-2015-06-649087. Epub 2015 Aug. 28. PMID: 26320100

### Lysine-Specific Demethylase 1 is a Therapeutic Target for Fetal Hemoglobin Induction.

Shi L, Cui S, Engel J D, Tanabe O.
Nat Med. 2013 March; 19(3):291-4. doi: 10.1038/nm.3101. Epub 2013 Feb. 17. PMID: 23416702

### CORUM: The Comprehensive Resource of Mammalian Protein Complexes-2009.

Giurgiu M, Reinhard J, Brauner B, Dunger-Kaltenbach I, Fobo G, Frishman G, Montrone C, Ruepp A
Nucleic Acids Res. 2010 January; 38(Database issue):D497-501. doi: 10.1093/nar/gkp914. Epub 2009 Nov. 1. Pmid: 19884131

### Densely Interconnected Transcriptional Circuits Control Cell States in Human Hematopoiesis

Novershtern N, Subramanian A, Lawton L. N, Raymond H. M, Haining N, McConkey M. E, Habib N, Yosef N, Chang C. Y, Shay T, Frampton C. M, Drake A. C. B, Leskov I, Nilsson B, Preffer F, Dombkowski D, Evans J. W, Liefeld R, Smutko J. S, Chen J, Friedman N, Young R. A, Golub T. R, Regev A, Ebert B. L.
Cell. 2011 Jan. 21; 144(2):296-309. doi: 10.1016/j.cell.2011.01.004. PMID: 21241896
The Genotype-Tissue Expression (GTEx) project
The GTEx Consortium
Nat Genet. 2013 June; 45(6):580-5. doi: 10.1038/ng.2653. PMID: 23715323

All publications and patent applications described herein are hereby incorporated by reference in their entireties.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A method for increasing expression of a fetal hemoglobin (HbF) in a cell, optionally a eukaryotic cell, comprising contacting a cell with an inhibitor of a target protein or target protein complex that functions to regulate HbF expression, optionally wherein the target protein is Cullin 3 (CUL3) or Speckle-type POZ protein (SPOP).

2. The method of claim 1, wherein the target protein is CUL3.

3. The method of claim 1 wherein the target protein is SPOP.

4. The method of any one of claims 1-3, wherein the HbF comprises hemoglobin gamma and hemoglobin alpha.

5. The method of claim 4, wherein the hemoglobin gamma comprises hemoglobin gamma G1 (HBG1) and/or or hemoglobin gamma G2 (HBG2).

6. The method of any one of claims 1-5, wherein the target protein or protein complex regulates HbF expression via a molecular signaling pathway listed in Table 5.

7. The method of claim 6, wherein the molecular signaling pathway is selected from the group consisting of: glucagon signaling pathway, carbon metabolism, oxytocin signaling, glycolysis, gluconeogenesis, endocrine resistance, Gonadotropin-releasing hormone (GnRH) signaling, oocyte meiosis, fatty acid degradation, and inflammatory mediator regulation of Transient Receptor Potential (TRP) channels.

8. The method of any one of claims 1-7, wherein the target protein is selected from those listed in Table 1 or Table 2.

9. The method of any one of claims 1-8, wherein the target protein is permanently or transiently associated with a multi-protein complex that regulates HbF expression.

10. The method of claim 9, wherein the multi-protein complex is selected from those listed in Table 3 or Table 4.

11. The method of claim 9 or claim 10, wherein CUL3 is permanently or transiently associated with the multi-protein complex.

12. The method of claim 11, wherein the multi-protein complex is selected from D(4) dopamine receptor (DRD4)-Kelch like protein 12 (KLH12)-CUL3, ubiquitin E3 ligase, coiled coil domain containing protein 22 (CCDC22)-COMM domain containing protein 8 (COMMD8)-CUL3, or Cullin associated NEDD8 dissociated protein (CAND1)-CUL3-E3 ubiquitin protein ligase RBX1 (RBX).

13. The method of claim 9 or claim 10, wherein SPOP is permanently or transiently associated with the multi-protein complex.

14. The method of claim 13, wherein the multi-protein complex is a ubiquitin E3 ligase complex.

15. The method of any one of claims 1-14, wherein the inhibitor targets or binds a nucleotide sequence encoding the target protein or a protein in the protein complex, thereby inhibiting or preventing the expression of the target protein or a protein in the protein complex.

16. The method of claim 15, wherein the nucleotide sequence encoding the target protein or the protein in the protein complex is DNA.

17. The method of claim 15, wherein the nucleotide sequence encoding the target protein or the protein in the protein complex is RNA.

18. The method of claim 17, wherein the nucleotide sequence encodes CUL3, and optionally comprises or consists of a nucleic acid encoding the amino acid sequence of SEQ ID NO: 108 or an antisense sequence thereof.

19. The method of claim 17, wherein the nucleotide sequence encodes SPOP, and optionally comprises or consists of a nucleic acid encoding the amino acid sequence of SEQ ID NO: 109 or an antisense sequence thereof.

20. The method of any one of claims 1-19, wherein the inhibitor is selected from the group consisting of: a small molecule, a nucleic acid, a polypeptide, and a nucleoprotein complex.

21. The method of claim 20, wherein the nucleic acid is selected from the group consisting of: DNA, RNA, shRNA, siRNA, microRNA, gRNA, and antisense oligonucleotide.

22. The method of claim 20, wherein the polypeptide is selected from the group consisting of: a protein, a peptide, a protein mimetic, a peptidomimetic, an antibody or functional fragment thereof, and an antibody-drug conjugate or a functional fragment thereof.

23. The method of claim 20, wherein the nucleoprotein complex is a ribonucleoprotein complex (RNP) comprising:
a) a first sequence comprising a guide RNA (gRNA) that specifically binds a target sequence, wherein the target sequence comprises a regulator of HbF expression and
b) a second sequence encoding a CRISPR-Cas protein
wherein the CRISPR-Cas protein comprises a DNA-nuclease activity.

24. The method of any one of claims 1-23, wherein the cell is a blood cell.

25. The method of claim 24, wherein the blood cell is an erythrocyte.

26. The methods of any one of claims 1-25, wherein the contacting a cell occurs in vitro, in vivo, ex vivo, or in situ.

27. A pharmaceutical composition for increasing expression of fetal hemoglobin (HbF) in a subject in need thereof, comprising:
an inhibitor of a target protein or protein complex that functions to regulate HbF expression, and
a diluent, excipient, and carrier
wherein the composition is formulated for delivery to a subject in need thereof.

28. The pharmaceutical composition of claim 27, wherein the inhibitor is a small molecule.

29. The pharmaceutical composition of claim 28, wherein the small molecule inhibitor targets CUL3.

30. The pharmaceutical composition of claim 29, wherein the CUL3 small molecule inhibitor is selected from the group consisting of: MLN4924, suramin, and DI-591.

31. The pharmaceutical composition of claim 27, wherein the inhibitor is a nucleic acid.

32. The pharmaceutical composition of claim 31, wherein the nucleic acid is selected from DNA, RNA, shRNA, siRNA, microRNA, gRNA, and antisense oligonucleotide.

33. The pharmaceutical composition of claim 27, wherein the inhibitor is a polypeptide.

34. The pharmaceutical composition of claim 33, wherein the polypeptide is selected from a protein, a peptide, a protein mimetic, a peptidomimetic, an antibody or functional fragment thereof, and an antibody-drug conjugate or a functional fragment thereof.

35. The pharmaceutical composition of any one of claims 33-34, wherein the polypeptide specifically binds a regulator of HbF expression.

36. The pharmaceutical composition of claim 27, wherein the inhibitor is a ribonucleoprotein (RNP) complex comprising:
a) a first sequence comprising a guide RNA (gRNA) that specifically binds a target sequence, wherein the target sequence comprises a regulator of HbF expression and
b) a second sequence encoding a CRISPR-Cas protein
wherein the CRISPR-Cas protein comprises a DNA-nuclease activity.

37. The pharmaceutical composition of claim 36, wherein the gRNA binds a gene encoding the regulator of HbF expression.

38. The pharmaceutical composition of claim 36, wherein the target sequence is listed in any one of Tables 1, 3-4, and 6-7.

39. The pharmaceutical composition of claim 38, wherein the target sequence is CUL3.

40. The pharmaceutical composition of claim 38, wherein the target sequence is SPOP.

41. The pharmaceutical composition of claim 37, wherein the gRNA comprises any one of the sequences disclosed in Table 2 or a fragment thereof, or an antisense sequence of any of the foregoing.

42. The pharmaceutical composition of claim 41, wherein the gRNA binds a gene encoding CUL3, and optionally comprises or consists of GAGCATCTCAAACACAACGA (SEQ ID NO: 94), CGAGATCAAGTTGTACGTTA (SEQ ID NO: 95), or TCATCTACGGCAAACTCTAT (SEQ ID NO: 96).

43. The pharmaceutical composition of claim 41, wherein the gRNA binds a gene encoding SPOP, and optionally comprises or consists of TAACTTTAGCTTTTGCCGGG (SEQ ID NO: 91), CGGGCATATAGGTTTGTGCA (SEQ ID NO: 92), or GTTTGCGAGTAAACCCCAAA (SEQ ID NO: 93).

44. The pharmaceutical composition of claim 36 or claim 37, wherein the first sequence comprising the gRNA comprises a sequence encoding a promoter capable of expressing the gRNA in a eukaryotic cell.

45. The pharmaceutical composition of claim 36 or claim 37, wherein the second sequence comprising the CRISPR-Cas protein comprises a sequence capable of expressing the CRISPR-Cas protein in a eukaryotic cell.

46. The method of any of claims 1-26 or the pharmaceutical composition of claim 44 or claim 45, wherein the eukaryotic cell is a mammalian cell.

47. The method of any of claims 1-26 or the pharmaceutical composition of any one of claims 44-46, wherein the eukaryotic cell is a blood cell.

48. The method of any of claims 1-26 or the pharmaceutical composition of any one of claims 44-46, wherein the eukaryotic cell is an erythrocyte.

49. The method of any one of claims 1-26, wherein the inhibitor is delivered via a vector.

50. The method of claim 49, wherein the vector is a viral vector.

51. The method of claim 50, wherein the viral vector comprises a sequence isolated or derived from an adeno-associated virus (AAV).

52. A method of treating a disease or disorder associated with a defect in a hemoglobin protein activity or expression, comprising providing to a subject in need thereof the composition of any one of claims 27-51.

53. The method of claim 52, wherein the disease or disorder is a blood disorder.

54. The method of claim 53, wherein the blood disorder is selected from a group consisting of: Sickle cell disease, β-thalassemia, β-thalessemia intermedia, β-thalessemia major, β-thalessemia minor, and Cooley's anemia.

55. The method of any one of claims 52-54, wherein the hemoglobin protein is selected from hemoglobin-alpha and hemoglobin-beta.

56. The method of any one of claims 52-55, wherein the defect in the hemoglobin protein activity or expression results from a mutation, substitution, deletion, insertion, frameshift, inversion, or transposition to a nucleotide sequence which encodes the hemoglobin protein.
